(19) United States
(12) Patent Application Publication
(10) Pub. No.: US 2023/0225944 A1
(43) Pub. Date: Jul. 20, 2023

(54) DEVICES AND METHODS FOR A VIAL ADAPTER ASSEMBLY

(71) Applicant: Regeneron Pharmaceuticals, Inc., Tarrytown, NY (US)

(72) Inventors: Bryan GRYGUS, Clifton Park, NY (US); Daniel HALBIG, Ballston Lake, NY (US); Eric HOUDE, Queensbury, NY (US); Prithvi SINGH, Dorchester, MA (US); Paige WAECHTER, Dorchester, MA (US); Parker VALDEZ, Mountain View, CA (US); Sandy SPENCER, West Seneca, NY (US); Sindhuja KUCHIBHATLA, Albany, NY (US); Trevor LANGLEY, Rensselaer, NY (US)

(73) Assignee: Regeneron Pharmaceuticals, Inc., Tarrytown, NY (US)

(21) Appl. No.: 18/155,324

(22) Filed: Jan. 17, 2023

Related U.S. Application Data
(60) Provisional application No. 63/300,554, filed on Jan. 18, 2022.

(51) Int. Cl. A61J 1/20

(52) U.S. Cl. CPC A61J 1/2096; A61J 1/2089; A61J 1/2051

(57) ABSTRACT

A vial adapter assembly that includes a housing, a first receptacle formed within the housing and configured to receive a first vial within the housing, and a second receptacle formed within the housing and configured to receive a second vial within the housing. An opening of the first receptacle and an opening of the second receptacle is in fluid communication with the upper surface of the housing. The vial adapter assembly includes a cap that is coupled and movable relative to the housing from a closed configuration to an open configuration. In the closed configuration, the cap is configured to cover the opening of the first receptacle and the opening of the second receptacle on the upper surface. In the open configuration, the cap is configured to uncover the opening of the first receptacle and the opening of the second receptacle, and expose the upper surface.

### CROSS-REFERENCE TO RELATED APPLICATIONS

This application claims the benefit of priority from U.S. Provisional Application 63/300,554, filed Jan. 18, 2022, the entirety of which is incorporated herein by reference.

### FIELD OF DISCLOSURE

Aspects of the present disclosure relate to devices and methods for assembling and controlling access to the contents of multiple containers storing a medicament. More specifically, embodiments of the present disclosure relate to devices and methods for co-packaging, storing, transporting, and/or facilitating access to multiple (two or more) vials storing different drug substances or other fluids together in a single housing.

### INTRODUCTION

Drug products including fluid drug substances may be deliverable to patients in a variety of ways, including via injection. In many cases, a fluid drug substance is loaded and stored in a container, such as a vial. To preserve the fluid drug substance and prevent its contamination prior to use, fluid drug substances may be stored in separate vials. In some instances, a procedure may require administration of, e.g., one or more injections of, a solution that comprises a combination of multiple fluid drug substances that cannot be formulated into together into a single vial. In this instance, each of the fluid drug substances are individually stored in separate vials. For example, certain immunotherapies (e.g., immuno-oncology therapies) may involve an administration of a composition or formulation of different fluid substances that cannot be formulated into a single vial but that must be administered together, e.g., in a single injection. Human error in individually maintaining, handling, controlling, or administering multiple vials storing different fluid substances may impact the injection's efficacy in a patient and the subsequent clinical effect on the patient.

Additional aspects of fluid drug delivery can complicate the goal of accurately delivering a correct combination of different fluid substances via injection. For example, for a correct mixture of drug substances to be administered to a patient via a device (e.g., a syringe), a packaging, labeling, and/or storage of multiple loaded vials must be performed without error, human or otherwise.

### SUMMARY

Disclosed herein are combination devices, and particularly vial adapters, for attaching, co-packaging, and controlling access to the contents of two or more vials. In one embodiment of the present disclosure, a vial adapter assembly includes a housing and a first receptacle formed within the housing and configured to at least partially receive a first vial within a body of the housing. An opening of the first receptacle is in fluid communication with an upper surface of the housing. The vial adapter assembly includes a second receptacle formed within the housing, and configured to at least partially receive a second vial within the body of the housing. An opening of the second receptacle is in fluid communication with the upper surface of the housing. The vial adapter assembly includes a cap coupled to the housing, wherein the cap is movable relative to the housing from a closed configuration to an open configuration. In the closed configuration, the cap is configured to cover the opening of the first receptacle and the opening of the second receptacle on the upper surface of the housing. In the open configuration, the cap is configured to uncover the opening of the first receptacle and the opening of the second receptacle, and expose the upper surface of the housing.

In some aspects of the present disclosure, the housing includes at least one slot on the upper surface of the housing, and the cap includes at least one corresponding protrusion extending outwardly from a surface of the cap configured to mate with the upper surface of the housing. In the closed configuration, each of the at least one slots is configured to receive one of the at least one corresponding protrusions. In the open configuration, each of the at least one protrusions is configured to deform, such that the cap is inhibited from transitioning from the open configuration to the closed configuration. The first receptacle incudes a second opening along a lower portion of the housing, and the second receptacle incudes a second opening along the lower portion. The first openings of the first and second receptacles are sized smaller relative to the second openings of the first and second receptacles. The second openings of the first receptacle and the second receptacle are sized to receive the first vial and the second vial, respectively. The first openings of the first receptacle and the second receptacle are positioned to align with first and second openings of the first and second vial, respectively, when the first and second vial are received within the first and second receptacles.

In some aspects of the present disclosure, the first receptacle is positioned adjacent to the second receptacle such that the first vial is maintained alongside the second vial when received within the housing. The vial adapter assembly further includes a single hinge coupling the housing to the cap, wherein the hinge is configured to pivot the cap relative to the housing when moving from the closed configuration to the open configuration. The cap is configured to simultaneously cover the opening of the first receptacle and the opening of the second receptacle when in the closed configuration, and simultaneously uncover the opening of the first receptacle and the opening of the second receptacle when in the open configuration. The housing is configured to at least partially enclose the first vial and the second vial such that the first vial and the second vial are positioned adjacent to one another. The vial adapter assembly incudes an actuator positioned over an opening of the first receptacle and an opening of the second receptacle. The actuator includes a first hollow piercing element extending towards the first receptacle and a second hollow piercing element extending towards the second receptacle. The actuator is configured to simultaneously move the first hollow piercing element relative to the first receptacle and the second hollow piercing element relative to the second receptacle.

According to another embodiment of the present disclosure, a vial adapter assembly includes a housing, a first receptacle formed within the housing, and configured to at least partially receive a first vial, a second receptacle formed within the housing, and configured to at least partially receive a second vial, and a first actuator coupled to the housing and positioned in longitudinal alignment with the first receptacle, such that the first actuator is positioned over an opening of the first receptacle. The first actuator includes a hollow piercing element extending towards the first receptacle. The vial adapter assembly includes a second actuator coupled to the housing and positioned in longitudinal alignment with the second receptacle, such that the second actuator is positioned over an opening of the second receptacle. The second actuator includes a hollow piercing element extending towards the second receptacle. Each of the first actuator and the second actuator are independently movable relative to the housing between an extended configuration and a compressed configuration. In the extended configuration, the piercing element of the first actuator and the piercing element of the second actuator are positioned outside of the first receptacle and second receptacle, respectively, such that the piercing element of the first actuator and the piercing element of the second actuator are not in fluid communication with the first receptacle and the second receptacle. In the compressed configuration, the piercing element of the first actuator and the piercing element of the second actuator are positioned at least partially within the first receptacle and the second receptacle, respectively, such that the piercing element of the first actuator and the piercing element of the second actuator are in fluid communication with the first receptacle and the second receptacle, respectively.

In some aspects of the present disclosure, the vial adapter assembly further includes a first removable barrier coupled to the housing and positioned between the first actuator and the first receptacle, and a second removable barrier coupled to the housing and positioned between the second actuator and the second receptacle. The first removable barrier and the second removable barrier include an adhesive liner. The housing includes a first slot and a second slot, and the first slot and the second slot are sized to receive at least one of the first removable barrier and the second removable barrier.

In some aspects of the present disclosure, the first removable barrier is configured to inhibit movement of the first actuator from the extended configuration to the compressed configuration when the first removable barrier is received within the first slot. The second removable barrier is configured to inhibit movement of the second actuator from the extended configuration to the compressed configuration when the second removable barrier is received within the second slot. The first removable barrier and the second removable barrier are configured to be independently removable relative to the housing. Each of the first actuator and the second actuator include a luer lock connector.

In a further embodiment of the present disclosure, a vial adapter assembly includes a housing, a first receptacle formed within the housing, and configured to at least partially receive a first vial, and a first connector fixed to the housing and positioned at least partially within the first receptacle so that a distal end of the first connector extends within and is in fluid communication with the first vial when received within the first receptacle. The vial adapter assembly includes a second receptacle formed within the housing, and configured to at least partially receive a second vial, and a second connector fixed to the housing and positioned at least partially within the second receptacle so that a distal end of the second connector extends within and is in fluid communication with the second vial when received within the second receptacle.

The vial adapter assembly includes a first actuator coupled to the housing and positioned in longitudinal alignment with the first receptacle, and a second actuator coupled to the housing and positioned in longitudinal alignment with the second receptacle. Each of the first actuator and the second actuator are independently movable relative to the housing between an extended configuration and a compressed configuration. When the first actuator and the second actuator are in the extended configuration, the first actuator and the second actuator conceal a proximal region of the first connector and the second connector, respectively. When the first actuator and the second actuator are in the compressed configuration, the first actuator and the second actuator expose the proximal region of the first connector and the second connector, respectively.

In some aspects of the present disclosure, the vial adapter assembly further includes a first removable barrier coupled to the first actuator, and a second removable barrier coupled to the second actuator when the first actuator and the second actuator are in the extended configuration. The first removable barrier and the second removable barrier include at least one of an adhesive liner or a cap. The first removable barrier is positioned over a proximal end of the first connector when coupled to the first actuator, and the second removable barrier is positioned over a proximal end of the second connector when coupled to the second actuator. The vial adapter assembly further includes a first lock-out mechanism configured to prevent the first actuator from returning from the compressed configuration to the extended configuration, and a second lock-out mechanism configured to prevent the second actuator from returning from the compressed configuration to the extended configuration.

In some aspects of the present disclosure, the proximal region of the first connector extends outwardly from the first actuator when the first actuator is in the compressed configuration, and the proximal region of the second connector extends outwardly from the second actuator when the second actuator is in the compressed configuration. The first removable barrier and the second removable barrier are configured to be independently movable relative to the housing. The proximal region of each of the first connector and the second connector include a luer lock.

In a further embodiment of the present disclosure, a vial adapter assembly includes a housing and a first receptacle formed within the housing. The first receptacle is configured to receive a first vial therein. The vial adapter assembly includes a first connector movably coupled to the housing and positioned at least partially within the first receptacle, and the first connector includes a first channel defining a first flow path. The vial adapter assembly includes a second receptacle formed within the housing, wherein the second receptacle is configured to receive a second vial therein. The vial adapter assembly includes a second connector movably coupled to the housing and positioned at least partially within the second receptacle, and the second connector includes a second channel defining a second flow path. The first connector and the second connector are each configured to independently move relative to the housing between a first configuration and a second configuration. In the first configuration, the first flow path is obstructed, and the second flow path is obstructed. In the second configuration, the first flow path is unobstructed, and the second flow path is unobstructed.

In some aspects of the present disclosure, the vial adapter assembly further includes a first stopper extending around a distal region of the first connector within the first receptacle, and a second stopper extending around a distal region of the second connector within the second receptacle. The first flow path extends through a sidewall of a distal region of the first connector, and wherein the second flow path extends through a sidewall of a distal region of the second connector. The vial adapter assembly further includes a first stopper extending around the distal region of the first connector within the first receptacle, and a second stopper extending around the distal region of the second connector within the second receptacle. A portion of the first stopper is configured to obstruct the first flow path when the first connector is in the first configuration, and the second stopper is configured to obstruct the second flow path when the second connector is in the first configuration.

In some aspects of the present disclosure, the first connector is configured to rotate relative to the housing when transitioning from the first configuration to the second configuration. The second connector is configured to rotate relative to the housing when transitioning from the first configuration to the second configuration. The first connector and the second connector are each configured to independently rotate relative to the housing between the first configuration and the second configuration. Each of the first connector and the second connector include a proximal region that extends proximately out from the housing, and is configured to be independently movable relative to the housing. Each of the first connector and the second connector include a proximal region configured to couple to an external device. The proximal region configured to couple to the external device includes a luer lock.

In another embodiment of the present disclosure, a vial adapter assembly comprises a housing; a first receptacle formed within the housing and configured to at least partially receive a first vial within a body of the housing, wherein an opening of the first receptacle is in fluid communication with an upper surface of the housing; a cap coupled to the housing, wherein the cap includes a hollow piercing element and the cap is movable relative to the housing from a closed configuration to an open configuration; wherein, in the closed configuration, the cap is configured to cover the opening of the first receptacle on the upper surface of the housing; and wherein, in the open configuration, the cap is configured to uncover the opening of the first receptacle and expose the upper surface of the housing; and an external device coupled to the housing.

In some aspects of the present disclosure, the vial adapter assembly includes a second receptacle formed within the housing, and the vial adapter assembly is configured to at least partially receive a second vial within the body of the housing, wherein an opening of the second receptacle is in fluid communication with the upper surface of the housing. The hollow piercing element extends distally from an interior surface of the cap. The cap includes a proximal region configured to couple to the external device. The proximal region configured to couple to the external device includes a connector extending proximally from an exterior surface of the cap. The connector is a luer lock. The external device is a syringe or an IV bag.

In a further embodiment of the present disclosure, a vial adapter assembly comprises a housing; a first cartridge within the housing, the first cartridge containing a first substance; a second cartridge within the housing, the second cartridge containing a second substance; a first actuator coupled to the housing and positioned in longitudinal alignment with the first cartridge; a second actuator coupled to the housing and positioned in longitudinal alignment with the second cartridge, wherein each of the first actuator and the second actuator are independently movable relative to the housing between an extended configuration and a compressed configuration, wherein, in the extended configuration, a portion of the first actuator extends outwardly from the housing in a proximal direction and a portion of the second actuator extends outwardly from the housing in a proximal direction, and wherein, in the compressed configuration, a portion of the first actuator is concealed within the first cartridge and a portion of the second actuator is concealed within the second cartridge.

In some aspects of the present disclosure, the vial adapter assembly includes a third cartridge within the housing, the third cartridge containing a third substance, and a third actuator coupled to the housing and in longitudinal alignment with the third cartridge. In the extended configuration, a portion of the third actuator extends outwardly from the housing in a proximal direction and in the compressed configuration, a portion of the third actuator is concealed within the third cartridge. The housing includes a proximal end and a distal tip. A first actuation translates a distal end of the respective cartridge towards the distal tip of the housing and a second actuation delivers the substance stored within the respective cartridge. The first actuation translates the distal end of the respective cartridge outward from the housing via an opening of the distal tip. The distal tip of the housing is configured to connect to an external device. The distal tip includes a luer lock connection. The external device is a syringe or an IV bag. The housing includes a first receptacle including the first cartridge and a second receptacle including the second cartridge. The first cartridge is a first syringe and the second cartridge is a second syringe. The first syringe includes a first needle extending from a distal end of the first syringe and the second syringe includes a first needle extending from a distal end of the second syringe.

### BRIEF DESCRIPTION OF THE DRAWINGS

The accompanying drawings, which are incorporated into and constitute a part of this specification, illustrate various exemplary embodiments and, together with the description, serve to explain principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements in various embodiments, other than those specifically shown, are contemplated and are within the scope of the present disclosure.

There are many embodiments described and illustrated herein. The vial adapters and methods are neither limited to any single aspect nor embodiment thereof, nor to any combinations and/or permutations of such aspects and/or embodiments. Moreover, each of the aspects of the described embodiments thereof, may be employed alone or in combination with one or more of the other aspects of the described inventions and/or embodiments thereof. For the sake of brevity, certain permutations and combinations are not discussed and/or illustrated separately herein.

FIG. 1 depicts an exemplary vial adapter according to some embodiments of the present disclosure.

FIG. 2 depicts another exemplary vial adapter and components thereof, according to additional embodiments of the present disclosure.

FIG. 3 depicts a cross-section of the vial adapter depicted in FIG. 2.

FIGS. 4A-4B depict cross-sections of the vial adapter depicted in FIG. 2 in a partially actuated and a fully actuated state, respectively, according to aspects of the present disclosure.

FIG. 5 depicts a further exemplary vial adapter according to additional embodiments of the present disclosure.

FIGS. 6-8 depict cross sections of the vial adapter depicted in FIG. 5 in unactuated, partially actuated, and actuated states, respectively, according to aspects of the present disclosure.

FIG. 9 depicts a further exemplary vial adapter according to additional embodiments of the present disclosure.

FIGS. 10-11 depict cross-sections of the vial adapter depicted in FIG. 9 in closed and opened configurations, respectively, according to aspects of the present disclosure.

FIGS. 12-13 depict side and top views, respectively, of a further exemplary vial adapter according to additional embodiments of the present disclosure.

FIGS. 14-15 depict a further exemplary vial adapter and components thereof, according to additional embodiments of the present disclosure.

FIG. 16 depicts a further exemplary vial adapter according to additional embodiments of the present disclosure.

FIG. 17 depicts a portion of the vial adapter depicted in FIG. 16 attached to a mouth of a vial, according to aspects of the present disclosure.

FIG. 18 depicts a further exemplary vial adapter according to additional embodiments of the present disclosure.

FIGS. 19A-19B depict a further exemplary vial adapter in different stages of assembly, according to additional embodiments of the present disclosure.

FIG. 20 depicts a top view of the exemplary vial adapter depicted in FIGS. 19A-19B.

FIG. 21 depicts a side view of an exemplary double vial according to additional embodiments of the present disclosure.

FIG. 22 depicts a top view of the exemplary double vial depicted in FIG. 21.

FIGS. 23-24 depict side and top views, respectively, a further exemplary vial adapter according to additional embodiments of the present disclosure.

FIGS. 25-28 depict a further exemplary vial adapter and components thereof, according to additional embodiments of the present disclosure.

FIGS. 29-30 depict a side view and a cross-section, respectively, of a further exemplary vial adapter and components thereof, according to additional embodiments of the present disclosure.

FIGS. 31A-31B depict the vial adapter depicted in FIG. 29-30 in unactuated and actuated states, respectively, according to aspects of the present disclosure.

FIGS. 32A-32B depict unactuated and actuated states, respectively, of a further exemplary vial adapter and method of assembling said vial adapter, according to additional embodiments of the present disclosure.

FIGS. 33A-33B depict unaccessed and accessed states of a further exemplary vial adapter, according to additional embodiments of the present disclosure.

FIG. 34 depicts a further exemplary vial adapter, according to additional embodiments of the present disclosure.

FIGS. 35A-35B depict cross sections of different states of an exemplary embodiment of the vial adapter depicted in FIG. 34, according to aspects of the present disclosure.

FIGS. 36A-36B depict an exemplary adapter in an unassembled and an assembled state, according to additional embodiments of the present disclosure.

FIGS. 37A-37B depict a further exemplary adapter in unassembled and assembled states, according to additional embodiments of the present disclosure.

FIG. 38 depicts a further exemplary adapter, according to additional embodiments of the present disclosure.

FIG. 39 depicts a cross-section of a further exemplary adapter, according to additional embodiments of the present disclosure.

FIG. 40 depicts an exemplary syringe, according to additional embodiments of the present disclosure.

FIG. 41 depicts a cross-section of a further exemplary vial adapter, according to additional embodiments of the present disclosure.

FIG. 42 depicts an exemplary syringe, according to additional embodiments of the present disclosure.

FIG. 43 depicts a further exemplary vial adapter, according to additional embodiments of the present disclosure.

FIG. 44 depicts a portion of a further exemplary vial adapter, according to additional embodiments of the present disclosure.

FIG. 45 depicts an exemplary crimp assembly, according to additional embodiments of the present disclosure.

FIG. 46 depicts a further exemplary crimp assembly, according to additional embodiments of the present disclosure.

There are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

### DETAILED DESCRIPTION

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Notably, an embodiment or implementation described herein as an “example” or “exemplary” is not to be construed as preferred or advantageous, for example, over other embodiments or implementations; rather, it is intended reflect or indicate the embodiment(s) is/are one “example,” rather than “ideal.” In addition, the terms “first,” “second,” and the like, herein do not denote any order, quantity, or importance, but rather are used to distinguish an element, a structure, a step or a process from another. Moreover, the terms “a” and “an” herein do not denote a limitation of quantity, but rather denote the presence of one or more of the referenced items. Additionally, the terms “about,” “approximately,” “substantially,” and the like, when used in describing a numerical value, denote a variation of +/−10% of that value, unless specified otherwise.

Embodiments of the present disclosure may be used with any type of fluid-containing products, such as liquid drug substances, liquid placebos, or other liquids that may be dispensed in a dose form. As used herein, the term “drug substance” may refer to a formulated substance including an active ingredient or ingredients. In some embodiments, devices and aspects of the present disclosure can be used with any therapies using the immune system to combat diseases, such as immunotherapies, including vaccines, allergy treatments, cancer treatments, and more. In particular, devices and aspects of the present disclosure can be used for the treatment of patients with cancer (e.g., immune-oncology). However, it is also contemplated that embodiments of the present disclosure may be applicable to any therapy, liquid products, and/or any other context for which different substances may be stored separately in multiple vials. While medical indications are described herein, it is contemplated that non-medical liquids packaged together in different vials may be used in conjunction with the vial adapters described herein.

As used herein, the terms “proximal” and “proximally” refer to a location (or portion of a device) relatively closer to, or in the direction of, an access point, and the terms “distal” and “distally” refer to a location (or portion of a device) relatively closer to, or in the direction of, a user end opposite a proximal location/portion of a device.

As used herein, the term “body,” when used in reference to a part of a device, may refer to a component of the device suitable for containing a volume of a drug substance. A body may include, e.g., a barrel (such as a syringe barrel), tube, cylinder, or other containing portion of a device. In some embodiments, a body may also include a distal end portion having a nozzle, needle, needle attachment site, and/or distal cap.

In some embodiments, vial adapters according to the present disclosure may be manufactured, packaged, assembled with vials, and/or otherwise prepared according to processes relevant to the products (e.g., drug products) of which they may be a part. For example, in some embodiments, vial adapters according to the present disclosure may be packaged in, e.g., blister packaging, or may be assembled with filled vials and packaged in, e.g., blister packaging.

Described herein are various embodiments of vial adapters, and in particular, for co-packaging two or more vials and/or cartridges storing different contents. In some instances, embodiments or aspects of embodiments disclosed herein may be used in conjunction with existing vials, cartridges, and/or syringe body parts to modify off-the-shelf products. In other instances, embodiments or aspects of embodiments disclosed herein may be included in devices during their manufacture. Such parts may be intended to reduce human error and/or increase accuracy of administering a composition that will be formed by a therapy or mixture of multiple fluid substances stored in individual vials prior to administration, e.g., injection to a patient.

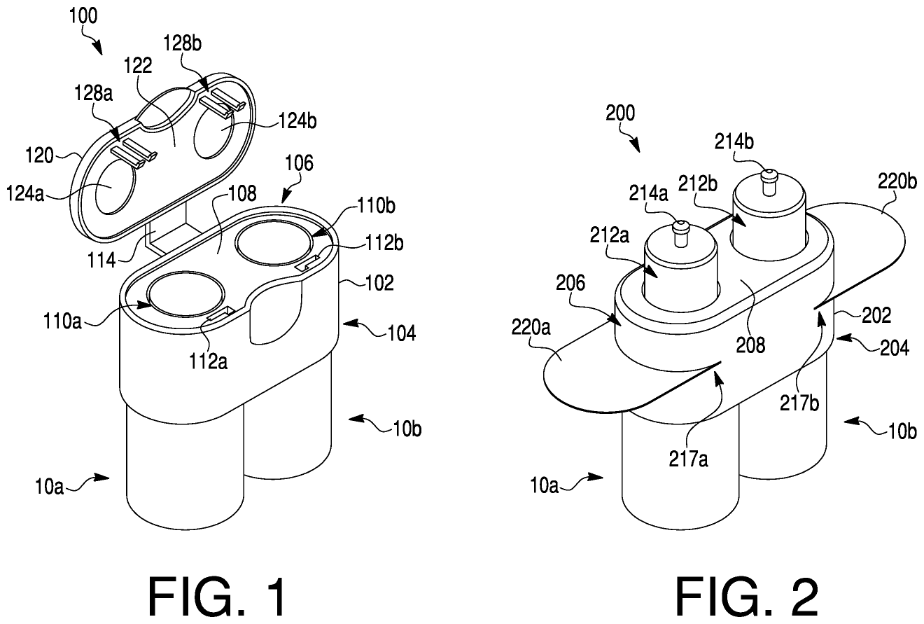

Referring now to FIG. 1, an exemplary vial adapter assembly 100 is depicted. Vial adapter assembly 100 may be configured and operable to retain and facilitate simultaneous access (fluid communication) to two or more vials housed together within separate receptacles upon the removal of a movable cap sealing each receptacle. Vial adapter assembly 100 may include a housing 102 having a distal end 104 and a proximal end 106. Vial adapter assembly 100 may include a plurality of receptacles formed within housing 102 and extending between the distal end 104 and the proximal end 106. Each of the plurality of receptacles may be configured to at least partially receive a vial within housing 102. In the example, vial adapter assembly 100 includes a pair of receptacles, such as a first receptacle for receiving a first vial 10a within a body of housing 102 and a second receptacle for receiving a second vial 10b within the body of housing 102. Although vial adapter assembly 100 is shown and described herein as including a pair of receptacles for receiving a pair of vials 10a, 10b, housing 102 may include additional receptacles for simultaneously storing additional vials adjacent to one another.

The first receptacle may include at least one opening along a lower portion of housing 102, such as proximate to or flush with distal end 104, which is sized and shaped to receive the first vial 10a within the first receptacle. The second receptacle may include at least one opening along the lower portion of housing 102 proximate to or flush with distal end 104 for receiving the second vial 10b therein. In some embodiments, the openings along the lower portion of housing 102 may be sized, shaped, and configured to form a frictional fit with each of the vials to thereby securely couple the first vial 10a and the second vial 10b to housing 102. In some aspects, portions of the first receptacle and the second receptacles may interact with a neck of the first vial 10a and a neck of the second vial 10b, respectively, to maintain the first vial 10a and the second vial 10b within the first receptacle and the second receptacle.

The first receptacle may be positioned adjacent to the second receptacle such that the first vial 10a is maintained alongside the second vial 10b when received within housing 102. It should be appreciated that the first and second receptacles may be sized and shaped relatively similar to one another such that the pair (or more) of receptacles may be configured to receive vials of similar cross-sectional dimensions. In other embodiments, the first and second receptacles may have varying cross-sectional dimensions relative to one another.

The pair of receptacles may be sized and shaped to at least partially receive a pair of vials, such that at least a portion of the first vial 10a and at least a portion of the second vial 10b may extend beyond housing 102. For example, the top portion of the vials 10a, 10b adjacent the vial opening may be disposed within housing 102 when the vials 10a, 10b are received by the receptacles. In this instance, the distal ends of the vials 10a, 10b may extend from the distal end 104 of housing 102 for ease of accessibility to a user of vial adapter assembly 100 to allow for visual inspection of the contents of the vials. Each of the pair of receptacles may include a corresponding opening 110a, 110b recessed from or flush with the proximal end 106, such that each of the openings 110a, 110b may be in fluid communication with an upper surface 108 of housing 102.

A first opening 110a of the first receptacle and a second opening 110b of the second receptacle are positioned along upper surface 108 to align with openings of the first and second vials 10a, 10b when the pair of vials 10a, 10b are received within the corresponding receptacles. Accordingly, openings 110a, 110b may allow access to first and second vials 10a, 10b, respectively when received within housing 102. In some embodiments, the openings 110a, 110b of the first and second receptacles along the upper surface 108 are sized relatively smaller than the openings of the first and second receptacles along the lower portion of housing 102.

Still referring to FIG. 1, vial adapter assembly 100 may include a cap 120 releasably coupled to housing 102, such that cap 120 may be movable relative to housing 102 from a closed configuration to an open configuration as depicted. Cap 120 may be coupled to housing 102 via a single hinge 114 that is configured to pivot cap 120 relative to housing 102 for transitioning vial adapter assembly 100 between the closed and open configurations. When in the closed configuration, cap 120 may be configured to simultaneously cover the pair of openings 110a, 110b along the upper surface 108 for each of the receptacles formed within housing 102 to seal and prevent access to openings 110a, 110b.

Each of housing 102 and cap 120 may include corresponding engagement mechanisms for fastening the components to one another when vial adapter assembly 100 is in the closed configuration. For example, housing 102 may include at least one slot along the upper surface 108 formed within housing 102, such as a first slot 112a for first receptacle and a second slot 112b for the second receptacle. Cap 120 may include at least one protrusion 128a, 128b extending outwardly from a bottom surface 122 of cap 120 for each slot 112a, 112b on housing 102. Protrusions 128a, 128b may be configured to mate with a corresponding slot 112a, 112b to facilitate a connection between cap 120 and housing 102 when vial adapter assembly 100 is in the closed configuration. Stated differently, when in the closed configuration, each of the slots 112a, 112b may be configured to receive a corresponding protrusion 128a, 128b to couple cap 120 to housing 102. Although one slot and protrusion for each receptacle is shown in FIG. 1, there may be any number of slot and receptacle pairings, for example one or more than two. Further, although the slot is described as being positioned on upper surface 108 and protrusions extending from bottom surface 122 of cap 120, the location of these structures may be switched.

The slot and protrusion configuration may allow cap 120 to serve a tamper-proof function to prevent tampering or re-use of the pair of vials 10a, 10b after cap 120 is transitioned from the closed configuration to the open configuration. For example, the slot and protrusion may be configured to prevent a user from closing cap 120 once it has been opened, thereby causing cap 120 to be maintained in a locked-out state. In another example, cap 120 may be weakly bonded (e.g., plastic-to-plastic welding or by an adhesive) to housing 102, such as, for example, by an adhesive positioned along proximal end 106. Cap 120 may be configured to disengage proximal end 106 upon applying a proximally-directed force to cap 120 that exceeds a bonding strength of the adhesive, thereby causing cap 120 to move proximally relative to housing 102. Upon disengaging cap 120 from housing 102, the adhesive positioned along proximal end 106 may be permanently deformed and/or damaged such that the bonding strength and/or properties of the adhesive may be diminished, thereby preventing cap 120 from returning to a locked state with housing 102. By preventing cap 120 from re-sealing to housing 102, tampering may be prevented.

In the example of FIG. 1, cap 120 may include a pair of protrusions 128a, 128b that are configured to interact with each slot 112a, 112b. A cross-sectional profile of the pair of protrusions 128a, 128b may be greater than a cross-sectional profile of the slots 112a, 112b, and the pair of protrusions 128a, 128b may be at least partially flexible. Pairs of protrusions 128a, 128b, may be deflected towards each other when received within each of the slots 112a, 112b and may be configured to expand when removed from slots 112a, 112b when cap 120 is opened to prevent a user from closing cap 120. Protrusions 128a, 128b may form a snap-fit connection with slots 112a, 112b to maintain vial adapter assembly 100 in the closed configuration until a minimum opening force is applied against cap 120 to extract the protrusions 128a, 128b from within the slots 112a, 112b. In other embodiments, other tamper-proof mechanisms may be incorporated in addition to or in place of the slot and protrusion arrangement described herein to prevent closure of cap 120 once it has been opened.

When in the closed configuration, cap 120 is configured to cover the upper surface 108 of housing 102. Cap 120 may include a lid 124a, 124b on the bottom surface 122 for each of the openings 110a, 110b on the upper surface 108 of housing 102. Each lid 124a, 124b may be sized and shaped to correspond with a cross-sectional dimension (e.g., diameter) of a corresponding opening 110a, 110b. Further, each lid 124a, 124b may be positioned relative to the bottom surface 122 to align with a position of the corresponding opening 110a, 110b on upper surface 108 when cap 120 is coupled to housing 102 in the closed configuration. Lids 124a, 124b may be configured to seal openings 110a, 110b in the closed configuration, thereby inhibiting contamination of the contents of each vial 10a, 10b when vial adapter assembly 100 is in the closed configuration.

As depicted in FIG. 1, when cap 120 of vial adapter assembly 100 is opened, cap 120 may be configured to simultaneously uncover the openings 110a, 110b of the first and second receptacles and uncover upper surface 108 of housing 102. As described above, each of the protrusions 128a, 128b may be configured to deform, e.g., radially outward, upon being removed from slots 112a, 112b, such that the protrusions 128a, 128b return to a cross-sectional profile that is greater than that of slots 112a, 112b. In this instance, cap 120 may be inhibited from transitioning from the open configuration to the closed configuration.

In use, the first vial 10a may be inserted within the first receptacle of vial adapter assembly 100, and the second vial 10b may be inserted within the second receptacle. Cap 120 may be coupled to housing 102, thereby concealing openings 110a, 110b to maintain a sterility of the contents stored in the first vial 10a and the second vial 10b. Vial adapter assembly 100 may then be packaged, shipped, stored, or otherwise manipulated while keeping the first vial 10a and the second vial 10b together. When a user is ready to access the contents of the first vial 10a and the second vial 10b, cap 120 may be transitioned from the closed configuration to the open configuration in response to pulling cap 120 proximally relative to upper surface 108, thereby causing the pair of protrusions 128a, 128b to disengage the corresponding slot 112a, 112a and allowing cap 120 to pivot about the single hinge 114.

In the open configuration, lower surface 122 of cap 120 is separated from upper surface 108 of housing 102, thereby exposing openings 110a, 110b and the proximal ends of the vials 10a, 10b received therein to facilitate simultaneous access to the contents stored therein. Accordingly, vial adapter assembly 100 may be operable to maintain the first vial 10a and the second vial 10b together for ease of handling, storage, and control before and during use. Housing 102 defines a sleeve positioned about the first vial 10a and the second vial 10b such that a user of vial adapter assembly 100 may be capable of simultaneously moving the pair of vials 10a, 10b in response to selectively maneuvering housing 102, and simultaneously accessing the contents of the vials 10a, 10b upon transitioning vial adapter assembly 100 from the closed configuration to the open configuration. By maintaining the first vial 10a and the second vial 10b proximate to one another before and during use, a likelihood of the vials being separated prior to administration or the contents of the vials not being administered together to a patient may be reduced. Vial adapter assembly 100 may be configured to provide a closed system for transferring fluid from each of the vials 10a, 10b to a corresponding external device received within the respective receptacle.

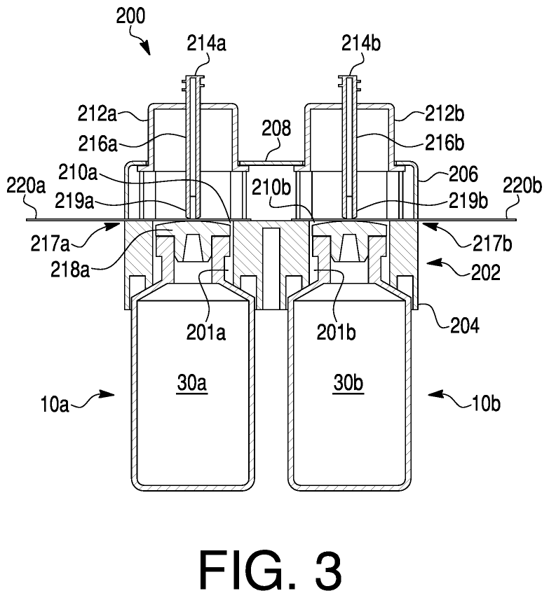

Referring now to FIG. 2, another exemplary vial adapter assembly 200 is depicted. Vial adapter assembly 200 may be configured and operable to retain and facilitate independent access (fluid communication) to two or more vials housed together within separate receptacles upon the removal of respective barriers sealing each receptacle. Vial adapter assembly 200 may include a housing 202 having a distal end 204 and a proximal end 206. Vial adapter assembly 200 may include a plurality of receptacles formed within housing 202 and extending between the distal end 204 and the proximal end 206. Each of the plurality of receptacles may be configured to at least partially receive one vial within housing 202. In the example, and as best seen in FIG. 3, vial adapter assembly 200 includes a pair of receptacles, such as a first receptacle 201a for receiving the first vial 10a within a body of housing 202 and a second receptacle 201b for receiving the second vial 10b within the body of housing 202.

Vial adapter assembly 200 may include one or more actuators coupled to housing 202 for each of the receptacles formed therein. In the example, vial adapter assembly 200 may include a first actuator 212a movably coupled to housing 202 and positioned in longitudinal alignment with an opening 210a of the first receptacle 201a. Vial adapter assembly 200 may include a second actuator 212b movably coupled to housing 202 and positioned in longitudinal alignment with an opening 210b of the second receptacle 201b.

Each of first actuator 212a and second actuator 212b may be independently movable relative to one another and housing 202, such as between an extended configuration and a compressed configuration. As described further below, each of first actuator 212a and second actuator 212b may be configured to establish fluid communication with the corresponding vial 10a, 10b received within the first and second receptacles 201a, 201b upon actuating (e.g., depressing) the respective actuator 212a, 212b.

Still referring to FIG. 2, first actuator 212a may include a first connector 214a and second actuator 212b may include a second connector 214b, each of which may be configured to couple the respective actuator 212a, 212b to an external device, such as a syringe. As described in detail herein, first actuator 212a may be configured to fluidly couple the first vial 10a to the external device (e.g., syringe) via the first connector 214a, and second actuator 212b may be configured to fluidly couple the second vial 10b to the external device (e.g., syringe) via the second connector 214b. In some embodiments, the external device coupled to first actuator 212a may be the same or different from the external device coupled to second actuator 212b. In the example, the first connector 214a and the second connector 214b may include a luer lock connector.

Vial adapter assembly 200 may include a first removable barrier 220a removably coupled to housing 202. In the example, housing 202 may include a first slot 217a positioned between distal end 204 and proximal end 206 of housing 202. The first slot 217a is sized, shaped, and configured to receive at least a portion of first removable barrier 220a therein. Vial adapter assembly 200 may further include a second removable barrier 220b removably coupled to housing 202. Housing 202 may include a second slot 217b positioned between distal end 204 and proximal end 206 along an opposite side of housing 202 from the first slot 217a, with the second slot 217b being sized, shaped, and configured to receive at least a portion of second removable barrier 220b therein. In some examples, one or more of first removable barrier 220a and/or the second removable barrier 220b may include an adhesive liner.

As best seen in FIG. 3, in an initial, default state of vial adapter assembly 200, first removable barrier 220a may be received within the first slot 217a and positioned between a distal end of first actuator 212a and the first receptacle 201a, and second removable barrier 220b may be received within the second slot 217b and positioned between a distal end of second actuator 212b and the second receptacle 201b. First removable barrier 220a may be configured to maintain sterility of the first vial 10a received within the first receptacle 201a, and second removable barrier 220b may be configured to maintain sterility of the second vial 10b received within the second receptacle 201b during packaging, storage, shipment, etc., of vials 10a, 10b within vial adapter assembly 200. Further, first removable barrier 220a extends across and covers opening 210a of the first receptacle 201a when received within slot 217a. This may prevent first actuator 212a from being depressed and moved into the compressed configuration. Likewise, second removable barrier 220b extends across and covers opening 210b of the second receptacle 201b when received within slot 217b. This may prevent second actuator 212b from being depressed and moved into the compressed configuration. Removable barriers 220a, 220b may serve a tamper-proof function to prevent re-use of the pair of vials 10a, 10b, because the presence of removable barriers 220a, 220b prevents premature actuation of first and second actuators 212a, 212b, and absence of the removable barriers 220a, 220b may indicate to a user that first and second actuators may have been actuated previously and/or first and second vials 10a, 10b may not be sterile.

First actuator 212a may include a first hollow piercing element 216a having a distal tip 219a that is aligned with and extends towards the first receptacle 201a when the vial adapter assembly 200 is in the initial, unactuated, default state. Second actuator 212b may include a second hollow piercing element 216b having a distal tip 219b that is aligned with and extends towards the second receptacle 201b when the vial adapter assembly 200 is in the initial, unactuated, default state. A channel extending through first hollow piercing element 216a may be in fluid communication with distal tip 219a at a distal end and first connector 214a at a proximal end, and a channel extending through second hollow piercing element 216b may be in fluid communication with distal tip 219b at a distal end and second connector 214b at a proximal end. Each hollow piercing element 216a, 216b is configured to move relative to housing 202 in response to an actuation of the corresponding actuator 212a, 212b. Hollow piercing element 216a may be coupled to actuator 212a so that when actuator 212a is depressed, hollow piercing element 216a is pushed downwards towards opening 210a of first receptacle 201a. Hollow piercing element 216b may be coupled to actuator 212b so that when actuator 212b is depressed, hollow piercing element 216b is pushed downwards towards opening 210b of second receptacle 201b.

Vial adapter assembly 200 may include a first stopper 218a disposed within first receptacle 201a and a second stopper 218b disposed within second receptacle 201b. Each of the stoppers 218a, 218b may be coupled to and/or at least partially received within the corresponding vial 10a, 10b received within the respective receptacle 201a, 201b. First stopper 218a may be configured to control access to or maintain sterility of a content 30a stored within first vial 10a received within first receptacle 201a, and second stopper 218b may be configured to control access to or maintain sterility of a content 30b stored within second vial 10b. The content 30a, 30b may include various substances, such as a medicament, fluid drug substance, or other liquid formulation. Further, as described above, content 30a, 30b may be different liquids from one another.

Still referring to FIG. 3, with first slot 217a positioned above (i.e., proximal to) receptacle opening 210a and first stopper 218a, first removable barrier 220a is configured to prevent depression of first actuator 212a and thus separate first hollow piercing element 216a from the first vial 10a when the first vial 10a is received within the first receptacle 201a and first removable barrier 220a is received within the first slot 217a. Accordingly, first removable barrier 220a may be configured to inhibit fluid communication between the channel of first hollow piercing element 216a and the first vial 10a by obstructing a path of distal tip 219a toward first stopper 218a.

Stated differently, first removable barrier 220a may be configured to inhibit movement of first actuator 212a from the extended configuration to the compressed configuration when first removable barrier 220a is received within the first slot 217a. In this instance, fluid communication between first actuator 212a and the first vial 10a is prevented by first removable barrier 220a when the first vial 10a is received within the first receptacle 201a.

Further, with second slot 217b positioned above (i.e., proximal to) receptacle opening 210b and second stopper 218b, second removable barrier 220b is configured to prevent depression of second actuator 212b and thus separate second hollow piercing element 216b from the second vial 10b when the second vial 10b is received within the second receptacle 201b and second removable barrier 220b is received within the second slot 217b. Accordingly, second removable barrier 220b may be configured to inhibit fluid communication between the channel of second hollow piercing element 216b and the second vial 10b by obstructing a path of distal tip 219b toward second stopper 218b. First and second removable barriers 220a, 220b may prevent accidental or premature depression of first and second actuators 212a, 212b. Further, first and second removable barriers 220a, 220b may act as a tamper-proof indicator so that if either of first or second removable barriers 220a, 220b is missing, a user may know to discard the contents of the corresponding vial, as they may have been compromised.

In other words, second removable barrier 220b may be configured to inhibit movement of second actuator 212b from the extended configuration to the compressed configuration when second removable barrier 220b is received within the second slot 217b. In this instance, fluid communication between second actuator 212b and the second vial 10b is prevented by second removable barrier 220b when the second vial 10b is received within the second receptacle 201b.

In exemplary use, first actuator 212a and second actuator 212b may be positioned above (i.e., proximal to) first receptacle 201a and second receptacle 201b, respectively, when in the extended configuration. In this instance, first and second actuators 212a, 212b may not be in fluid communication with first and second receptacles 201a, 201b, respectively. Each of first actuator 212a and second actuator 212b may be inhibited from being actuated (e.g., depressed) due to a position of first removable barrier 220a and second removable barrier 220b within the corresponding slot of housing 202.

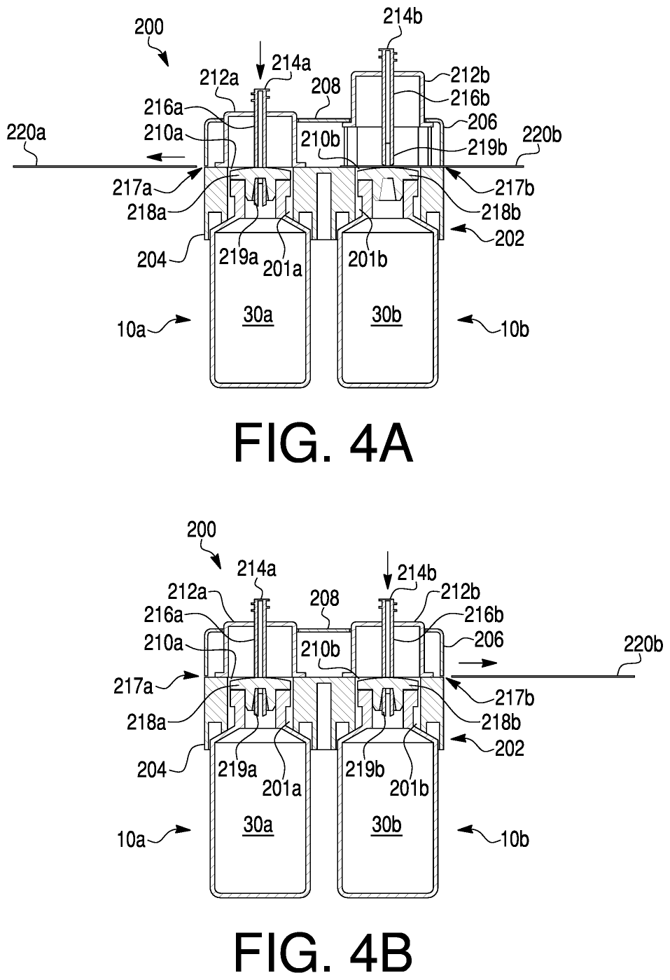

Accordingly, first actuator 212a is maintained in the extended configuration by first removable barrier 220a, and second actuator 212b is maintained in the extended configuration by second removable barrier 220b. First removable barrier 220a and second removable barrier 220b are configured to be independently removable from housing 202. Further, first actuator 212a and second actuator 212b may be independently movable relative to one another, such that a user of vial adapter assembly 200 may selectively and independently establish fluid communication between each of the external devices coupled to the pair of vials 10a, 10b via vial adapter assembly 200. In some aspects, once first and second actuators 212a, 212b have been actuated, a portion of first and second actuators 212a, 212b, or a portion of first and second receptacles 201a, 201b, may deform or otherwise engaged to lock first and second actuators 212a, 212b in an actuated configuration. This may prevent reuse of and/or tampering with vial adapter assembly 200 and thus promote the sterility of the contents of first and second vials 10a, 10b. As seen in FIG. 4A, first removable barrier 220a may be removed from within the first slot 217a to allow actuation of first actuator 212a, thereby transitioning first actuator 212a from the extended configuration to the compressed configuration. Fluid communication between first actuator 212a and the first vial 10a is permitted upon removal of first removable barrier 220a from the first slot 217a. For example, depressing first actuator 212a causes first hollow piercing element 216a to translate distally (e.g., downward) toward first receptacle 201a, through an opening in first stopper 218a, and into fluid communication with first vial 10a. In the compressed configuration, at least a portion of first actuator 212a is positioned at least partially inside first receptacle 201a.

Specifically, distal tip 219a may be configured to pierce first stopper 218a and extend into the first vial 10a, thereby establishing fluid communication between first connector 214a and the first vial 10a. An external device (e.g., a syringe) may be coupled to first connector 214a, e.g., a luer lock, to access the first fluid 30a via the channel of first hollow piercing element 216a.

As seen in FIG. 4B, second removable barrier 220b may be removed from within the second slot 217b to allow actuation of second actuator 212b, thereby transitioning second actuator 212b from the extended configuration to the compressed configuration. Fluid communication between second actuator 212b and the second vial 10b is permitted upon removal of second removable barrier 220b from the second slot 217b. For example, depressing second actuator 212b causes second hollow piercing element 216b to translate distally (e.g., downward) toward second receptacle 201b, through an opening in second stopper 218b, and into fluid communication with second vial 10b. In the compressed configuration, at least a portion of second actuator 212b is positioned at least partially inside second receptacle 201b.

Specifically, distal tip 219b may be configured to pierce second stopper 218b and extend into the second vial 10b, thereby establishing fluid communication between second connector 214b and the second vial 10b. An external device (e.g., a syringe) may be coupled to second connector 214b, e.g., a luer lock, to access the second fluid 30b via the channel of second hollow piercing element 216b.

In some embodiments, the first fluid 30a stored in the first vial 10a may be different than the second fluid 30b stored in the second vial 10b. In other embodiments, the first fluid 30a may be the same as the second fluid 30b. Further, the external device coupled to first actuator 212a via first connector 214a may be a first device that is different than a second device coupled to second actuator 212b via second connector 214b. In some embodiments, the same external device may be coupled to first actuator 212a and second actuator 212b, such as in series and/or simultaneously.

Accordingly, vial adapter assembly 200 may be operable to maintain the first vial 10a and the second vial 10b together for ease of handling, storage, and control before and during use. Housing 202 may define a sleeve positioned about the first vial 10a and the second vial 10b such that a user of vial adapter assembly 200 may be capable of simultaneously moving the pair of vials 10a, 10b in response to selectively maneuvering housing 202, and independently accessing the contents of the vials 10a, 10b upon transitioning the first and second actuators 212a, 212b of vial adapter assembly 200 between different states. By maintaining the first vial 10a and the second vial 10b proximate to one another before and during use, a likelihood of the vials being separated prior to administration or the contents of the vials not being administered together to a patient may be reduced.

Vial adapter assembly 200 may be configured to provide a closed system for transferring fluid from each of the vials 10a, 10b to a corresponding external device coupled to the respective connector 214a, 214b. In some embodiments, one or more of first actuator 212a and/or second actuator 212b may include a visual indicator (e.g., a color, a marking, etc.) that may allow a user to distinguish between the two vials in the two respective receptacles. In some aspects, the visual indicator of first actuator 212a and/or second actuator 212b may be associated with a corresponding indicia on the first vial 10a and/or the second vial 10b, respectively. The visual indicator of the actuator 212a, 212b may facilitate a visual identification of the content (e.g., medicament) stored in each vial 10a, 10b, thereby providing a visual reference to a user of vial adapter assembly 200 of the content being delivered from each receptacle 201a, 201b during use.

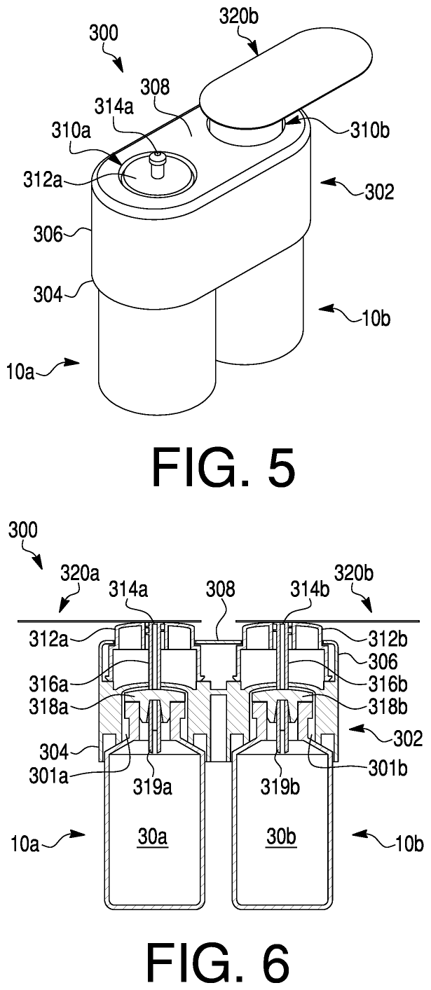

Referring now to FIG. 5, another exemplary vial adapter assembly 300 is depicted. Vial adapter assembly 300 may be configured and operable to retain and facilitate independent access (fluid communication) to two or more vials housed together within separate receptacles upon the removal of respective barriers sealing each access point. In vial adapter assembly 200, transitioning the first and second actuators 212a, 212b to the compressed configuration caused first and second hollow piercing elements 216a, 216b to extend through first and second stoppers 218a, 218b to establish fluid communication with first and second vials 10a, 10b. By contrast, vial adapter assembly 300 includes first and second hollow piercing elements 316a, 316b that are always in fluid communication with first and second vials 10a, 10b, and first and second actuators 312a, 312b alternatively prevent or allow access to the contents of the vials, as is described further below.

Vial adapter assembly 300 may include a housing 302 having a distal end 304, a proximal end 308, and a sidewall 306 extending between the distal end 304 and the proximal end 308. Vial adapter assembly 300 may include a plurality of receptacles formed within housing 302 and extending between the distal end 304 and the proximal end 308. Each of the plurality of receptacles may be configured to at least partially receive one vial within housing 302. In the example, and as best seen in FIG. 6, vial adapter assembly 300 includes a pair of receptacles, such as a first receptacle 301a for receiving the first vial 10a within a body of housing 302 and a second receptacle 301b for receiving the second vial 10b within the body of housing 302.

Vial adapter assembly 300 may include an actuator coupled to housing 302 for each of the receptacles formed therein. In the example, vial adapter assembly 300 may include a first actuator 312a movably coupled to housing 302 and positioned in longitudinal alignment with the first receptacle 301a. Vial adapter assembly 300 may include a second actuator 312b movably coupled to housing 302 and positioned in longitudinal alignment with the second receptacle 301b.

Each of first actuator 312a and second actuator 312b may be independently movable relative to one another and housing 302, such as between an extended configuration and a compressed configuration. As described further below, each of first actuator 312a and second actuator 312b may be configured to allow access to the corresponding vial 10a, 10b received within the first and second receptacles 301a, 301b upon actuating (e.g., depressing) the respective actuator 312a, 312b.

Still referring to FIGS. 5 and 6, first actuator 312a may include a first connector 314a, and second actuator 312b may include a second connector 314b, each of which may be configured to couple the respective first and second connector 314a, 314b to an external device, such as a syringe. As described in detail herein, first connector 314a may be configured to fluidly couple the first vial 10a to the external device (e.g., syringe), and second connector 314b may be configured to fluidly couple the second vial 10b to the external device (e.g., syringe). In some embodiments, the external device coupled to first connector 314a may be the same or different from the external device coupled to second connector 314b. In the example, the first connector 314a and the second connector 314b may include a luer lock connector.

In the example, first connector 314a may be fixed relative to housing 302 and positioned at least partially within first receptacle 301a. First connector 314a may extend within and establish fluid communication with the first vial 10a when received within first receptacle 301a. As described in further detail below, first actuator 312a may be configured to move relative to first connector 314a such that a position of first connector 314a remains fixed when first actuator 312a moves from an extended configuration to a compressed configuration. As described further herein, when first actuator 312a is in an extended configuration, first actuator 312a may surround first connector 314a and prevent an external device from coupling to first connector 314a. When first actuator 312a is depressed, first actuator 312a may move downward in a distal direction to reveal first connector 314a, which may then extend upwards beyond first actuator 312a in the compressed configuration.

Likewise, second connector 314b may be fixed relative to housing 302 and positioned at least partially within second receptacle 301b. Second connector 314b may extend within and establish fluid communication with the second vial 10b when received within second receptacle 301b. Second actuator 312b may be configured to move relative to second connector 314b such that a position of second connector 314b remains fixed when second actuator 312a moves from an extended configuration to a compressed configuration. As described further herein, when second actuator 312b is in an extended configuration, second actuator 312b may surround second connector 314b and prevent an external device from coupling to second connector 314b. When second actuator 312b is depressed, second actuator 312b may move downward in a distal direction to reveal second connector 314b, which may then extend upwards beyond second actuator 312b when in the compressed configuration. It should be understood that FIG. 5 depicts first actuator 312a in the compressed configuration and second actuator 312b in the extended configuration (and with a removable barrier coupled thereto) for illustrative purposes.

As best seen in FIG. 6, a first removable barrier 320a may be removably coupled to vial adapter assembly 300. First removable barrier 320a may be positioned to cover a proximal surface of first actuator 312a, and over a proximal end of first connector 314a surrounded by first actuator 312a in the extended configuration. A portion of first removable barrier 320a may extend out from the proximal surface of the first actuator 312a to facilitate grasping first removable barrier 320a to remove it from first actuator 312a. A pulling or peeling force, for example, may be applied to remove first removable barrier 320a from first actuator 312a.

Similarly, vial adapter assembly 300 may further include a second removable barrier 320b removably coupled to second actuator 312b, and over a proximal end of second connector 314b surrounded by second actuator 312b in the extended configuration, as described in regards to first removable barrier 320a relative to first actuator 312a. In some examples, one or more of first removable barrier 320a and/or the second removable barrier 320b may include an adhesive liner, a cap, and/or other suitable devices for covering the actuators.

Still referring to FIG. 6, in an initial, default state of vial adapter assembly 300, first actuator 312a and second actuator 312b are positioned in the extended configuration. First actuator 312a is configured to conceal first connector 314a when in the extended configuration, and second actuator 312b is configured to conceal second connector 314b when in the second configuration. Additionally, first removable barrier 320a may be coupled to first actuator 312a, thereby at least partially concealing first connector 314a, when first actuator 312a is in the extended configuration. Second removable barrier 320b may be coupled to second actuator 312b, thereby at least partially concealing second connector 314b, when second actuator 312b is in the extended configuration.

First removable barrier 320a may be configured to maintain sterility between first connector 314a and the first vial 10a received within the first receptacle 301a by covering first actuator 312a and first connector 314a, and second removable barrier 320b may be configured to maintain sterility between second connector 314b and the second vial 10b received within the second receptacle 301b by covering second actuator 312b and second connector 314b. First removable barrier 320a and second removable barrier 320b may further inhibit depression of first actuator 312a and second actuator 312b when coupled to first actuator 312a and second actuator 312b, respectively. To depress first actuator 312a and second actuator 312b, first removable barrier 320a and second removable barrier 320b must first be removed. As described in detail herein, when each of first actuator 312a and second actuator 312b are in the compressed configuration, first connector 314a and second connector 314b may be revealed and accessible to an external device.

First connector 314a may be fluidly coupled to a first hollow piercing element 316a having a distal tip 319a that is aligned with and extends into the first receptacle 301a when the vial adapter assembly 300 is in the initial, extended state. Second connector 314b may be fluidly coupled to a second hollow piercing element 316b having a distal tip 319b that is aligned with and extends into the second receptacle 301b when the vial adapter assembly 300 is in the initial, extended state. A channel of first hollow piercing element 316a may be in fluid communication with distal tip 319a at a distal end and with first connector 314a at a proximal end, and a channel of second hollow piercing element 316b may be in fluid communication with distal tip 319b at a distal end and second connector 314b at a proximal end. Each connector 314a, 314b and hollow piercing element 316a, 316b is fixed relative to housing 302 such that the corresponding actuators 312a, 312b are configured to move relative to connectors 314a, 314b and hollow piercing elements 316a, 316b in response to an actuation of the actuators 312a, 312b.

Vial adapter assembly 300, or the vials 10a, 10b positioned within first and second receptacles 301a, 301b, may include a first stopper 318a and a second stopper 318b. Each of the stoppers 318a, 318b may be coupled to and/or at least partially received within an opening of the corresponding vial 10a, 10b received within the respective receptacle 301a, 301b. First stopper 318a may be configured to maintain sterility of the first content 30a stored within first vial 10a received within first receptacle 301a, and second stopper 318b may be configured to maintain sterility of the second content 30b stored within second vial 10b. The first content 30a and the second content 30b may include various substances, such as a medicament, fluid drug substance, or other liquid formulation, which may be the same or different from one another.

Still referring to FIG. 6, first removable barrier 320a disposed over first actuator 312a is configured to inhibit movement of first actuator 312a from the extended configuration to the compressed configuration. In the example, first connector 314a may be disposed within a body of first actuator 312a when first actuator 312a is in the extended configuration, such that first connector 314a is obstructed from coupling with an external device. Accordingly, first removable barrier 320a is configured to inhibit an external device (e.g., a syringe) from connecting to first connector 314a and establishing fluid communication with the first vial 10a received within the first receptacle 301a.

Further, second removable barrier 320b disposed over second actuator 312b is configured to inhibit movement of second actuator 312b from the extended configuration to the compressed configuration. In the example, second connector 314b may be disposed within a body of second actuator 312b when second actuator 312b is in the extended configuration, such that second connector 314b is obstructed from coupling with an external device. Accordingly, second removable barrier 320b is configured to inhibit an external device (e.g., a syringe) from connecting to second connector 314b and establishing fluid communication with the second vial 10b received within the second receptacle 301b. While first and second connectors 314a, 314b may be in fluid communication with first and second vials 10a, 10b, respectively, in both the extended and compressed configurations, an external device may not be able to fluidly connect with first and second connectors 314a, 314b in the extended configuration because first and second actuators 312a, 312b block access to the connectors. First and second actuators 312a, 312b must be moved out of the way (e.g., depressed) in order to access first and second connectors 314a, 314b.

In exemplary use, first actuator 312a and second actuator 312b may be positioned in the extended configuration, with first removable barrier 320a attached to an exterior surface of first actuator 312a and second removable barrier 320b attached to an exterior surface of second actuator 312b. In this instance, first and second connectors 314a, 314b may be disposed within the first and second actuators 312a, 312b, respectively. Accordingly, first and second connectors 314a, 314b may be prevented from connecting with an external device, and each of first and second actuators 312a, 312b may be inhibited from being actuated (e.g., depressed) due to a position of first and second removable barriers 320a, 320b, respectively.

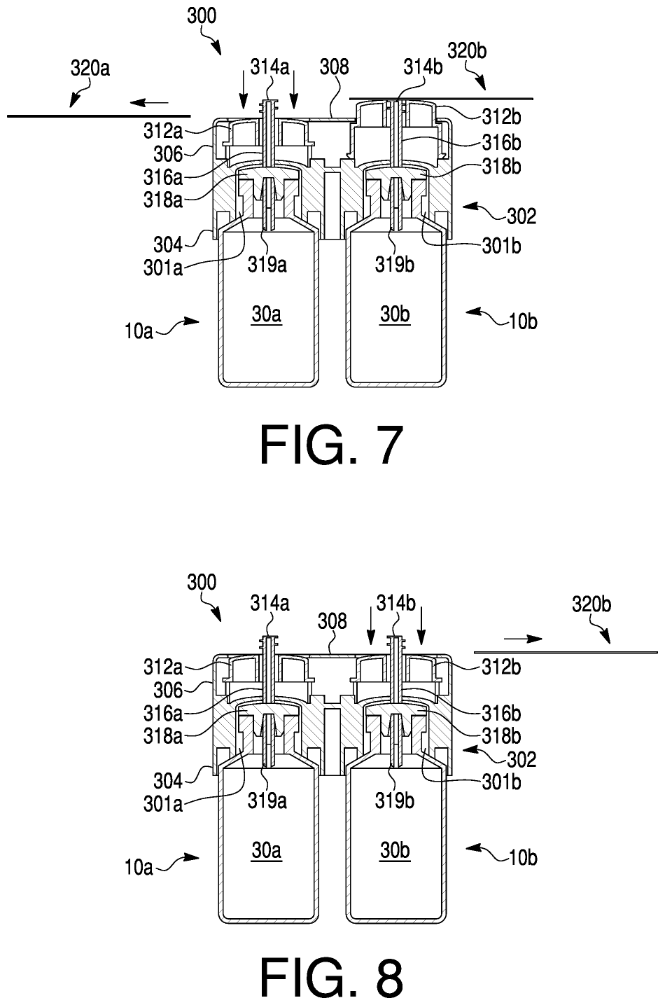

In some embodiments, housing 302 may include one or more tamper proof mechanisms for preventing re-use of vial adapter assembly 300 and/or preventing first actuator 312a and second actuator 312b from inadvertently moving from the extended configuration to the compressed configuration. For example, each of first removable barrier 320a and second removable barrier 320b may include an adhesive liner positioned along an interior surface of the respective removable barrier, and the interior surface may be in contact with the corresponding actuator and connector when vial adapter assembly 300 is in the initial, default state (see FIG. 6). First removable barrier 320a may be configured to bond to first actuator 312a and first connector 314a, thereby inhibiting actuation (e.g. depression) of first actuator 312a from the extended configuration to the compressed configuration (see FIG. 7) until first removable barrier 320a is removed from first actuator 312a and first connector 314a. Second removable barrier 320b may be configured to bond to second actuator 312b and second connector 314b, thereby inhibiting actuation of second actuator 312b from the extended configuration to the compressed configuration (see FIG. 8) until second removable barrier 320b is removed from second actuator 312b and second connector 314b. Once removed, removable barriers 320a, 320b may not be able to be re-applied.

In another example, each of first actuator 312a and second actuator 312b may be configured to rotate relative to housing 302 and the corresponding connector. Upon removal of first removable barrier 320a, first actuator 312a may be inhibited from moving from the extended configuration to the compressed configuration. First, first actuator 312a may need to be rotated (e.g., clockwise, counterclockwise) relative to housing 302 to align with housing 302 to allow first actuator 312a to move to the compressed configuration. Similarly, upon removal of second removable barrier 320b, second actuator 312b may be inhibited from moving to the compressed configuration, and may need to be rotated relative to housing 302 to align with housing 302 prior to moving to the compressed configuration. Accordingly, housing 302 may be configured to inhibit actuation of each actuator 312a, 312b until each respective actuator 312a, 312b is independently rotated to align with the corresponding channel.

Accordingly, first actuator 312a is maintained in the extended configuration until first removable barrier 320a is detached from first actuator 312a, and second actuator 312b is maintained in the extended configuration until second removable barrier 320b is detached from second actuator 312b. First removable barrier 320a and second removable barrier 320b are configured to be independently removable relative to one another. Fluid communication between an external device and the first vial 10a is permitted upon removal of first removable barrier 320a from the first actuator 312a and depression of first actuator 312a, and fluid communication between an external device and the second vial 10b is permitted upon removal of second removable barrier 320b from the second actuator 312b and depression of first actuator 312b. Further, first actuator 312a and second actuator 312b may be independently movable relative to one another, such that a user of vial adapter assembly 300 may selectively establish fluid communication between each of the external devices coupled to the pair of vials 10a, 10b via vial adapter assembly 300.

As seen in FIG. 7, first removable barrier 320a may be detached from the exterior surface of first actuator 312a to allow actuation of first actuator 312a. In transitioning first actuator 312a from the extended configuration to the compressed configuration, first connector 314a may extend outwardly from first actuator 312a thereby exposing first connector 314a and allowing connection of an external device to vial adapter assembly 300. Accordingly, fluid communication between an external device and the first vial 10a is permitted upon removal of first removable barrier 320a and actuation of first actuator 312a. In the example, first hollow piercing element 316a is in fluid communication with first receptacle 301a with distal tip 319a positioned at least partially inside the first vial 10a when first actuator 312a is in the extended and compressed configurations. With first connector 314a exposed, an external device (e.g., a syringe) coupled to first connector 314a may access the first fluid 30a via the channel of first hollow piercing element 316a.

As seen in FIG. 8, second removable barrier 320b may be detached from the exterior surface of second actuator 312b to allow actuation of second actuator 312b. In transitioning second actuator 312b from the extended configuration to the compressed configuration, second connector 314b may extend outwardly from second actuator 312b thereby exposing second connector 314b and allowing connection of an external device to vial adapter assembly 300. Accordingly, fluid communication between an external device and the second vial 10b is permitted upon removal of second removable barrier 320b and actuation of second actuator 312b. In the example, second hollow piercing element 316b is in fluid communication with second receptacle 301b with distal tip 319b positioned at least partially inside the second vial 10b when second actuator 312b is in the extended and compressed configurations. With second connector 314b exposed, an external device (e.g., a syringe) coupled to second connector 314b may access the second fluid 30b via the channel of second hollow piercing element 316b.

In some embodiments, housing 302 may include one or more lock-out mechanisms for preventing first actuator 312a and second actuator 312b from returning to the extended configuration once the actuator is moved to the compressed configuration. For example, the lock-out mechanism may include one or more tabs, protrusions, fasteners, etc., that may preclude movement of first actuator 312a and second actuator 312b after moving to the compressed configuration. In some aspects, one or more portions of housing 302 and/or the corresponding receptacle 301a, 301b, of first actuator 312a and second actuator 312b may be configured to deform to lock the respective actuator 312a, 312b in the compressed configuration. In the example, housing 302 may include a first lock-out mechanism for independently fixing first actuator 312a in the compressed configuration relative to first receptacle 301a, and a second lock-out mechanism for independently fixing second actuator 312b in the compressed configuration relative to second receptacle 301b. The first and second lock-out mechanisms may serve a tamper-proof function and to prevent re-use of the pair of vials 10a, 10b after each of the actuators 312a, 312b are transitioned from the extended configuration to the compressed configuration, respectively.

Accordingly, vial adapter assembly 300 may be operable to maintain the first vial 10a and the second vial 10b together for ease of handling, storage, and control before and during use. Housing 302 may define a sleeve positioned about the first vial 10a and the second vial 10b such that a user of vial adapter assembly 300 may be capable of simultaneously moving the pair of vials 10a, 10b in response to selectively maneuvering housing 302, and independently accessing the contents of the vials 10a, 10b upon transitioning vial adapter assembly 300 between different states. By maintaining the first vial 10a and the second vial 10b proximate to one another before and during use, a likelihood of the vials being separated prior to administration or the contents of the vials not being administered together to a patient may be reduced.

Vial adapter assembly 300 may be configured to provide a closed system for transferring fluid from each of the vials 10a, 10b to a corresponding external device coupled to the respective connector 314a, 314b. In some embodiments, one or more of first actuator 312a and/or second actuator 312b may include a visual indicator (e.g., a color, a marking, etc.) that may allow a user to distinguish between the two vials associated with the respective connectors. In some aspects, the visual indicator of first actuator 312a and/or second actuator 312b may be associated with a corresponding indicia on the first vial 10a and/or the second vial 10b, respectively. The visual indicator of the actuator 312a, 312b may facilitate a visual identification of the content (e.g., medicament) stored in each vial 10a, 10b, thereby providing a visual reference to a user of vial adapter assembly 300 of the content being delivered from each receptacle 301a, 301b during use.

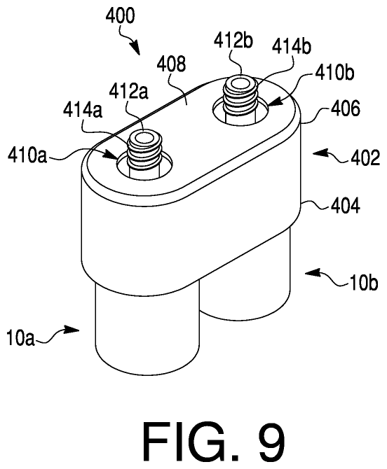

Referring now to FIG. 9, an exemplary vial adapter assembly 400 is depicted. Vial adapter assembly 400 may be configured and operable to retain and facilitate independent access (fluid communication) to two or more vials housed together within separate receptacles upon the actuation (e.g., rotation) of respective actuators coupled to each vial. Vial adapter assembly 400 may include a housing 402 having a distal end 404 and a proximal end 406. Vial adapter assembly 400 may include a plurality of receptacles formed within housing 402 and extending between the distal end 404 and the proximal end 406. Each of the plurality of receptacles may be configured to at least partially receive a vial within housing 402.

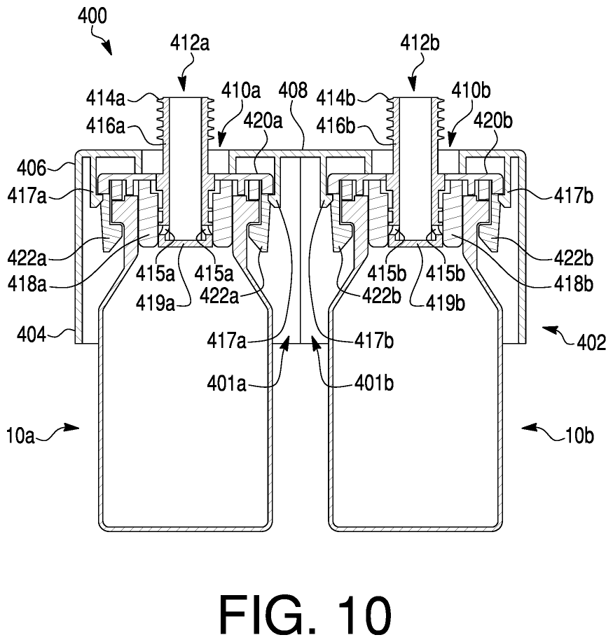

In the example, as best seen in FIG. 10, vial adapter assembly 400 includes a pair of receptacles, such as a first receptacle 401a for receiving the first vial 10a within a body of housing 402 and a second receptacle 401b for receiving the second vial 10b within the body of housing 402. Although vial adapter assembly 400 is shown and described herein as including a pair of receptacles for receiving a pair of vials 10a, 10b, housing 402 may include additional receptacles for simultaneously storing additional vials adjacent to one another.

First receptacle 401a may include at least one opening along a lower portion of housing 402, such as proximate to or flush with distal end 404, which is sized and shaped to receive the first vial 10a within first receptacle 401a. Second receptacle 401b may include at least one opening along the lower portion of housing 402 proximate to or flush with distal 404 for receiving the second vial 10b therein. As described herein, each of first receptacle 401a and second receptacle 401b may include an engagement interface for securing the corresponding vial 10a, 10b to housing 402.

Still referring to FIG. 10, first receptacle 401a may be positioned adjacent to second receptacle 401b such that the first vial 10a is maintained alongside the second vial 10b when received within housing 402. The pair of receptacles 401a, 401b may be sized and shaped to at least partially receive a pair of vials, such that at least a portion of the first vial 10a (e.g., the distal end) and at least a portion of the second vial 10b (e.g., the distal end) may extend outwardly from housing 402 for ease of accessibility or for visual inspection of the contents. Each of the pair of receptacles 401a, 401b may include a corresponding opening 410a, 410b near the proximal end 406, such that each of the openings 410a, 410b may be in fluid communication with an upper surface 408 of housing 102.

A first opening 410a of first receptacle 401a and a second opening 410b of second receptacle 401b are positioned along upper surface 408 to align with openings in the vials when the pair of vials 10a, 10b are received within the corresponding receptacles 401a, 401b. Vial adapter assembly 400 may further include a first connector 412a and a second connector 412b extending outwardly (e.g., proximally) from housing 402 through first opening 410a and second opening 410b, respectively.

Referring to FIGS. 9-10, first connector 412a may include a first engagement mechanism 414a for coupling first connector 412a to an external device, and second connector 412b may include a second engagement mechanism 414b for coupling second connector 412b to an external device. In the example, the first and second engagement mechanisms 414a, 414b may include a threaded portion positioned along an exterior surface of first and second connectors 412a, 412b, respectively. In other embodiments, connectors 412a, 412b may include various other suitable engagement mechanisms for facilitating connection to an external device, such as, for example, a clasp, a luer lock, a snap-fit connection, etc.

First connector 412a may be rotatably coupled to housing 402 and extend at least partially within first receptacle 401a, and second connector 412b may be rotatably coupled relative to housing 402 and may extend at least partially within second receptacle 401b. As described herein, each of first connector 412a and second connector 412b may be configured to move (e.g., rotate) relative to housing 402 and between a first (disconnected) configuration and a second (connected) configuration. Further, first connector 412a and second connector 412b may be independently movable relative to one another, such that a user of vial adapter assembly 400 may selectively establish fluid communication between external devices coupled to the pair of vials 10a, 10b via the respective connectors 412a, 412b.

As best seen in FIG. 10, first connector 412a may be coupled to a first bracket 420a, and second connector 412b may be coupled to a second bracket 420b. First bracket 420a may include a first pair of clips 422a that are configured to engage the proximal end of the first vial 10a, and second bracket 420b may include a pair of second clips 422b that are configured to engage the proximal end of the second vial 10b. A first stopper 418a may be sized and shaped to extend into the first vial 10a when first bracket 420a is coupled to the first vial 10a. First stopper 418a may be configured to receive at least a portion of a first hollow shaft 416a of first connector 412a within it.

As described herein, first hollow shaft 416a may be configured to move (e.g., rotate) relative to first stopper 418a in response to a corresponding movement (e.g., rotation) of first connector 412a. For example, a user may rotate first connector 412a using fingers or by connecting and rotating an external device (e.g., a syringe) coupled thereto. A second stopper 418b may be sized and shaped to extend into the second vial 10b when second bracket 420b is coupled to the second vial 10b. Second stopper 418b may be configured to receive at least a portion of a second hollow shaft 416b of second connector 412b within it. As further described herein, second hollow shaft 416b may be configured to move (e.g., rotate) relative to second stopper 418b in response to a corresponding movement (e.g., rotation) by second connector 412b. For example, a user may rotate second connector 412b using fingers or by connecting and rotating an external device (e.g., a syringe) coupled thereto.

First connector 412a and first hollow shaft 416a may be rotatable relative to first bracket 420a, first stopper 418a, the first vial 10a, and housing 402. Second connector 412b and second hollow shaft 416b may be rotatable relative to second bracket 420b, second stopper 418b, the second vial 10b, and/or housing 402.

Sill referring to FIG. 10, first hollow shaft 416a may include a closed distal end 419a extending into first vial 10a when first vial 10a is received within housing 402. First hollow shaft 416a may also include one or more openings 415a extending through a sidewall of first hollow shaft 416a near the closed distal end 419a. In a first (disconnected) configuration the one or more openings 415a may be positioned against an interior surface of first stopper 418a, and particularly a first portion of first stopper 418a. In this instance, the first portion of first stopper 418a may form a closed seal with the openings 415a.

When the openings 415a are aligned with the first portion of the first stopper 418a, first connector 412a is not in fluid communication with the first vial 10a. Accordingly, the contents of vial 10a received within first receptacle 401a may not be accessible via first connector 412a when openings 415a are blocked by the inner surface of first stopper 418a. In this way, first connector 412a may be configured to maintain a sterility of the first vial 10a when in the first (disconnected) configuration or to prevent access to the contents of first vial 10a.

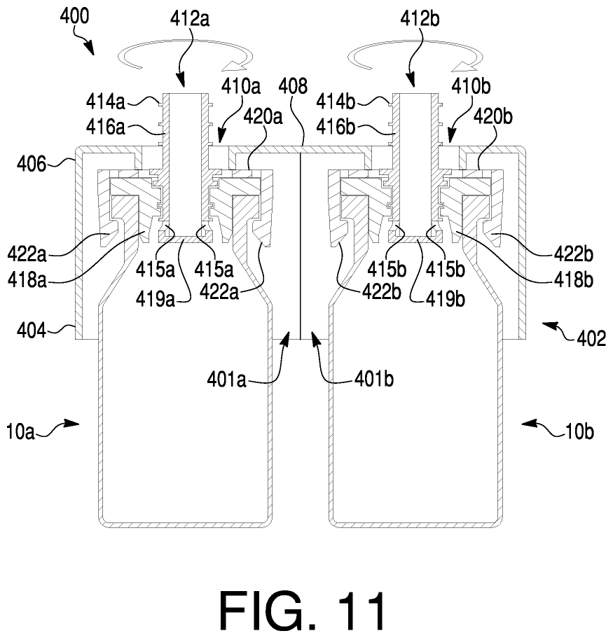

As described further herein and seen in FIG. 11, rotation of first connector 412a and first hollow shaft 416a from the first (disconnected) configuration to the second (connected) configuration may orient the one or more openings 415a towards a different interior surface of first stopper 418a, and particularly a second portion of first stopper 418a. The second portion of first stopper 418a includes a recessed portion, opening, or channel so that the second portion of stopper 418a is spaced apart from the first hollow shaft 416a. Accordingly, an opening of first stopper 418a may be wider at the second portion than at the first portion.

In this instance, the second portion of first stopper 418a may be spaced apart from the openings 415a so that the second portion does not obstruct openings 415a. With first connector 412a in the second (connected) configuration, the openings 415a may be unobstructed such that first connector 412a may be in fluid communication with the first vial 10a. In other words, the openings 415a may be aligned with the channel/opening/recessed portion of first stopper 418a such that first connector 412a is in fluid communication with the first vial 10a.

Still referring to FIG. 10, second connector 412b may include similar structure as first connector 412a, shown and described above, such that second connector 412b may be configured and operable like first connector 412a. For example, second connector 412b may include one or more openings 415b extending through a sidewall of second hollow shaft 416b near the closed distal end 419b. Further, second connector 412b and second hollow shaft 416b may be configured to transition from a first (disconnected) configuration to a second (connected) configuration to orient the one or more openings 415b from a first position being blocked by an interior surface of second stopper 418b to a second position in which the interior surface of second stopper 418b is not blocking the one or more openings 415b.

In exemplary use, each of the vials 10a, 10b may be coupled to the corresponding bracket 420a, 420b via engagement between the proximal neck portions of the vials 10a, 10b and the clips 422a, 422b of brackets 420a, 420b. Although brackets 420a, 420b and clips 422a, 422b are described in reference to FIGS. 10 and 11, any suitable mechanism for maintaining first and second vials 10a, 10b may be used. During use, first connector 412a may be coupled to an external device (e.g., a syringe), such as by mating the first engagement mechanism 414a (e.g., a luer lock) with a corresponding engagement feature (e.g., threaded portion) of the external device. Second connector 412b may be coupled to an external device (e.g., a syringe), such as by mating the second engagement mechanism 414b (e.g., a luer lock) with a corresponding engagement feature (e.g., threaded portion) of the external device.

Referring to FIGS. 10-11, the openings 415a on first hollow shaft 416a may be positioned within the first vial 10a and sealed by first stopper 418a such that first connector 412a is not in fluid communication with the first vial 10a when in the first (disconnected) configuration. Accordingly, the first hollow shaft 416a is fluidly disconnected from the first vial 10a, thereby inhibiting fluid flow between the external device coupled to first connector 412a and the first vial 10a. The openings 415b on second hollow shaft 416b may be positioned within the second vial 10b and sealed by second stopper 418b such that second connector 412b is not in fluid communication with the second vial 10b when in the first (disconnected) configuration. Accordingly, the second hollow shaft 416b is fluidly disconnected from the second vial 10b, thereby inhibiting fluid flow between the external device coupled to second connector 412b and the second vial 10b.

Referring to FIGS. 11, each of first actuator 412a and second actuator 412b may be independently rotated (e.g., by about 90 degrees) relative to housing 402 to transition from the first (disconnected) configuration to the second (connected) configuration. For example, rotation of the external device coupled to first actuator 412a may cause a corresponding rotation of first actuator 412a and first hollow shaft 416a relative to first stopper 418a disposed within the first vial 10a. Further, rotation of the external device coupled to second actuator 412b may cause a corresponding rotation of second actuator 412b and second hollow shaft 416b relative to second stopper 418b disposed within the second vial 10b.

Alternatively, rotation of housing 402 (e.g., by about 90 degrees) relative to first actuator 412a and/or second actuator 412b may similarly transition actuators 412a, 412b from the first (disconnected) configuration to the second (connected) configuration. In this instance, the openings 415a extending through sidewalls of first hollow shaft 416a may be realigned relative to first stopper 418a to permit fluid communication between first connector 412a and the first vial 10a. The openings 416b extending through sidewalls of second hollow shaft 416b may be realigned relative to second stopper 418b to permit fluid communication between second connector 412b and the second vial 10b.

Accordingly, vial adapter assembly 400 may be operable to maintain the first vial 10a and the second vial 10b together for ease of handling, storage, and control before and during use. Housing 402 defines a sleeve positioned about the first vial 10a and the second vial 10b such that a user of vial adapter assembly 400 may be capable of simultaneously moving the pair of vials 10a, 10b in response to selectively maneuvering housing 402, and independently accessing the contents of the vials 10a, 10b upon transitioning vial adapter assembly 400 from the first (disconnected) configuration to the second (connected) configuration. By maintaining the first vial 10a and the second vial 10b proximate to one another before and during use, a likelihood of the vials being separated prior to administration or the contents of the vials not being administered together to a patient may be reduced.

Vial adapter assembly 400 may be configured to provide a closed system for transferring fluid from each of the vials 10a, 10b to a corresponding external device coupled to the respective connector 412a, 412b. In some embodiments, one or more of first connector 412a and/or second connector 412b may include a visual indicator (e.g., a color, a marking, etc.) that may allow a user to distinguish between the two vials associated with the respective connector. In some aspects, the visual indicator of first connector 412a and/or second connector 412b may be associated with a corresponding indicia on the first vial 10a and/or the second vial 10b, respectively. The visual indicator of the actuator 412a, 412b may facilitate a visual identification of the content (e.g., medicament) stored in each vial 10a, 10b, thereby providing a visual reference to a user of vial adapter assembly 400 of the content being delivered from each receptacle 401a, 401b during use.

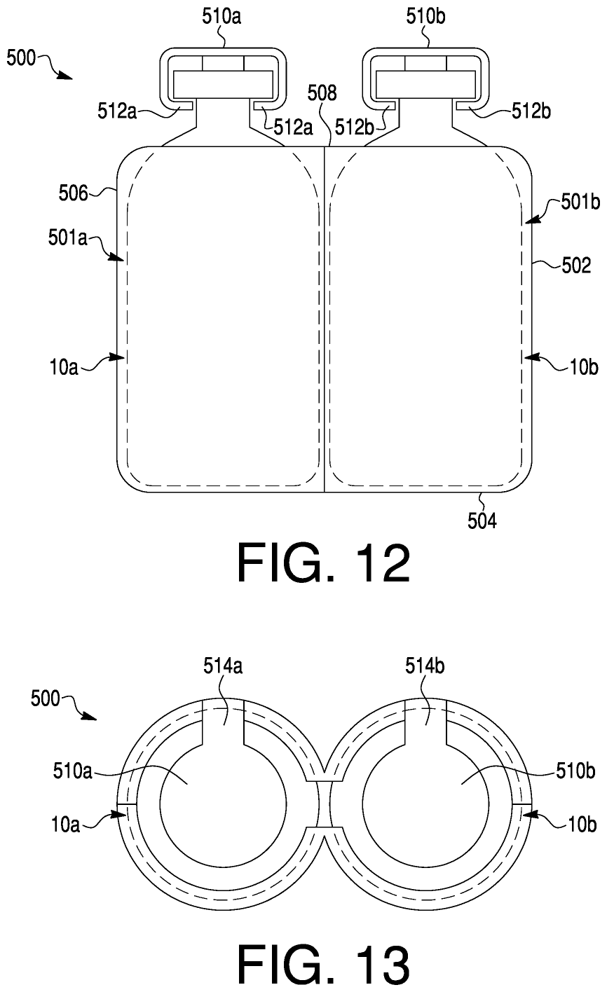

Referring now to FIG. 12, an exemplary vial adapter assembly 500 is depicted. Vial adapter assembly 500 may be configured and operable to retain and facilitate independent access (fluid communication) to two or more vials housed together within separate receptacles upon the removal of respective caps sealing each vial. Vial adapter assembly 500 may include a housing 502 having an at least partially closed distal end 504 and an at least partially open proximal end 506. Vial adapter assembly 500 may include a plurality of receptacles formed within housing 502 and extending between the closed distal end 504 and the open proximal end 506. Each of the plurality of receptacles may be configured to at least partially receive a vial within housing 502. In the example, vial adapter assembly 500 includes a pair of receptacles, such as a first receptacle 501a for receiving the first vial 10a within a body of housing 502 and a second receptacle 501b for receiving the second vial 10b within the body of housing 502.

The first and second receptacles 501a, 501b may each be configured to receive and retain the body of a vial within it, for example, from the shoulder of the vial downward. First receptacle 501a may be sized to permit the first vial 10a to extend outwardly (e.g., proximally) from the open proximal end 506 such that at least a mouth 19a of the first vial 10a is positioned above an upper surface 508 of housing 502 when vial adapter assembly 500 is in a first configuration. Second receptacle 501b may be sized to permit the second vial 10b to extend outwardly (e.g., proximally) from the open proximal end 506 such that at least a mouth 19b of the second vial 10b is positioned above upper surface 508 when vial adapter assembly 500 is in the first configuration. Although vial adapter assembly 500 is shown and described herein as including a pair of receptacles 501a, 501b for receiving a pair of vials 10a, 10b, housing 502 may include additional receptacles for simultaneously storing additional vials adjacent to one another.

Still referring to FIG. 12, vial adapter assembly 500 may include a first cap 510a and a second cap 510b. First cap 510a may be configured to engage a mouth 19a of the first vial 10a when the first vial 10a is received within first receptacle 501a. In the example, first cap 510a may include a periphery edge 512a that is configured to form a snap-fit connection with the underside of mouth 19a, thereby securely coupling the first vial 10a to the housing 502. First cap 510a may be sized, shaped, and configured to enclose an opening of the first vial 10a at the mouth 19a such that the first vial 10a is maintained in a closed state when first cap 510a is coupled to the mouth 19a. As described herein, first cap 510a may be configured to move independently of second cap 510b to disengage the mouth 19a, thereby transitioning the first vial 10a to an open state.

Second cap 510b may be configured to engage a mouth 19b of the second vial 10b when the second vial 10b is received within second receptacle 501b. In the example, second cap 510b may include a periphery edge 512b that is configured to form a snap-fit connection with the underside of mouth 19b, thereby securely coupling the second vial 10b to the housing 502. Second cap 510b may be sized, shaped, and configured to enclose an opening of the second vial 10a at the mouth 19b such that the second vial 10b is maintained in a closed state when second cap 510b is coupled to the mouth 19b. As described herein, second cap 510b may be configured to move independently of first cap 510a to disengage the mouth 19b, thereby transitioning the second vial 10b to an open state.

As seen in FIG. 13, vial adapter assembly 500 may include a first hinge 514a coupling first cap 510a to housing 502 such that first cap 514a may be configured to move (e.g., pivot) relative to housing 502 and between a coupled and decoupled state with the first vial 10a. Vial adapter assembly 500 may include a second hinge 514b coupling second cap 510b to housing 502 such that second cap 514b may be configured to move (e.g., pivot) relative to housing 502 and between a coupled and decoupled state with the second vial 10b.

FIG. 14 depicts a cap 510 of vial adapter assembly 500 detached from a mouth 19 of a vial 10 received within housing 502 in response to the periphery edge 512 disengaging the mouth 19 and the cap 510 pivoting away from the open proximal end 506 via the single hinge 514. It should be appreciated that the cap 510, periphery edge 512, and single hinge 514 shown in FIG. 14 may be representative of the caps 510a, 510b, periphery edges 512a, 512b, and hinges 514a, 514b shown and described in detail above.

Housing 502 may be formed of two or more separate sections that may be assembled to cooperatively enclose vials 10. In some embodiments, housing 502 may include at least one sidewall 505 that is selectively detachable from a reminder of housing 502 to facilitate receipt of the vial 10 within the corresponding receptacles. In the example, the at least one sidewall 505 may include one or more apertures and/or grooves 509 that are sized, shaped, and configured to mate with one or more protrusions and/or guides 503 on housing 502 for securely attaching the at least one sidewall 505 to the remaining housing 502 to enclose the vial 10. Although apertures and/or grooves 509 and protrusions and/or guides 503 are shown in FIG. 14, it should be understood that any suitable securing mechanism may be used to secure at least one sidewall 505 to the remaining housing 502. Further, separate sidewalls 505 may be separately attachable/detachable to the remaining housing 502 to independently remove or insert vials 10 into vial adapter assembly 500, or a single sidewall 505 may attach/detach to the remaining housing 502 to allow access to or enclosure of both vials 10 at the same time. When inserting vials 10 into vial adapter assembly 500, standard vial caps that come with the vials may be removed from vials 10 and replaced with caps 510a, 510b, or caps 510a, 510b may fit over and work with the vial caps that may come standard with the vials being inserted.

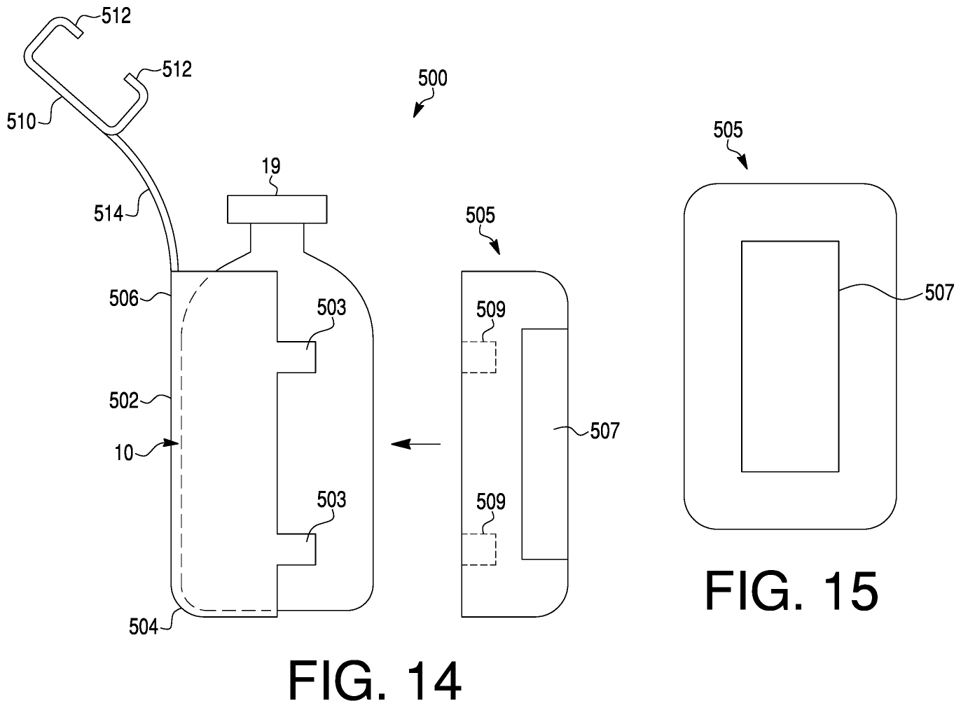

As seen in FIG. 15, the at least one sidewall 505 may include a window 507 configured to permit visual inspection of vial 10 within housing 502 when the at least one wall 505 is secured thereto. For example, a user of vial adapter assembly 500 may be permitted to visually inspect the contents of one or more vials 10 received within the corresponding receptacles of housing 502.

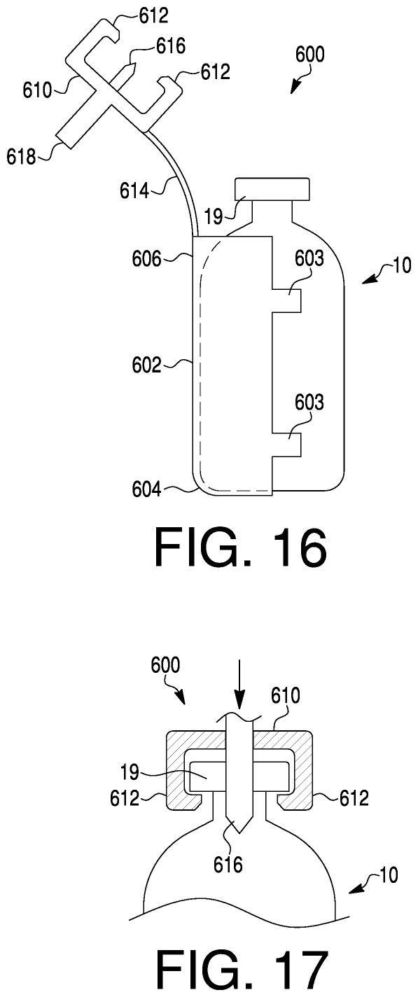

Referring now to FIG. 16, an exemplary vial adapter assembly 600 is depicted. Vial adapter assembly 600 may be configured and operable to retain and facilitate independent access (fluid communication) to two or more vials housed together within separate receptacles. Vial adapter assembly 600 may include a housing 602 having an at least partially closed distal end 604 and an at least partially open proximal end 606. Vial adapter assembly 600 may include a plurality of receptacles formed within housing 602 and extending between the closed distal end 604 and the open proximal end 606. Each of the plurality of receptacles may be configured to at least partially receive a vial within housing 602. In the example, vial adapter assembly 600 includes a pair of receptacles. Housing 602 of vial adapter assembly 600 may be similar to housing 502 of vial adapter assembly 500 described above.

Vial adapter assembly 600 may include a cap 610 for each of the pair of receptacles of housing 602. Cap 610 may be configured to engage a mouth 19 of the vial 10 when the vial 10 is received within the receptacle. In the example, cap 610 may include a periphery edge 612 that is configured to form a snap-fit connection with the underside of mouth 19, thereby securely coupling the cap 610 to the mouth 19. Cap 610 may be sized, shaped, and configured to enclose an opening of the vial 10 at the mouth 19 such that the vial 10 is maintained in a closed state when cap 610 is coupled to the mouth 19. As described herein, cap 610 may be configured to move independently of the other caps on housing 602 to disengage the mouth 19, thereby transitioning the vial 10 to an open state.

Still referring to FIG. 16, cap 610 may include a connector 618 extending proximally from an exterior surface of cap 610, and a needle 616 extending distally from an interior surface of cap 610. Connector 618 may be configured to facilitate access to vial 10 and/or movement of cap 610 between various positions relative to housing 602. In some aspects, connector 618 may include a luer lock or other connection mechanism to allow for needleless extraction of the contents of vial 10.

Needle 616 may have a sharp distal tip that is configured to pierce a lid cover 620 coupled to the mouth 19. Or, lid cover 620 that may come standard with the vial 10 may be removed and replaced with cap 610 when vial 10 is inserted into vial adapter assembly 600 and fitted with cap 610.

Still referring to FIG. 16, vial adapter assembly 600 may include a hinge 614 coupling cap 610 to housing 602 such that cap 610 may be configured to move (e.g., pivot) relative to housing 602 and between a coupled and decoupled state with the vial 10. In the coupled state, needle 616 may extend into vial 10 to facilitate access to the content of the vial, such as, for example, by an external device (e.g., a syringe).

FIG. 16 depicts cap 610 detached from the mouth 19 of the vial 10 received within housing 602. It should be appreciated that the cap 610, periphery edge 612, and hinge 614 may be configured and operable similar to similar components of vial adapter assembly 500 shown and described above except for the differences explicitly noted herein. In some embodiments, housing 602 may include at least one sidewall that is selectively detachable from a reminder of housing 602 to facilitate receipt of the vial 10 within the corresponding receptacles. The at least one sidewall may be configured to mate with one or more protrusions and/or guides 603 on housing 602 for securely attaching the at least one sidewall to the remaining housing 602 in a manner similar to vial adapter assembly 500.

As seen in FIG. 17, upon coupling cap 610 to the mouth 19 of the vial 10, the needle 616 may extend into the vial 10. With cap 610 in place, an external device may be coupled to connector 618 to access the contents of vial 10. For example, if connector 618 is a luer lock, a syringe body may be connected to connector 618 to fill the body of the syringe with the contents of the vial 10, or a connection to an IV bag may be established.

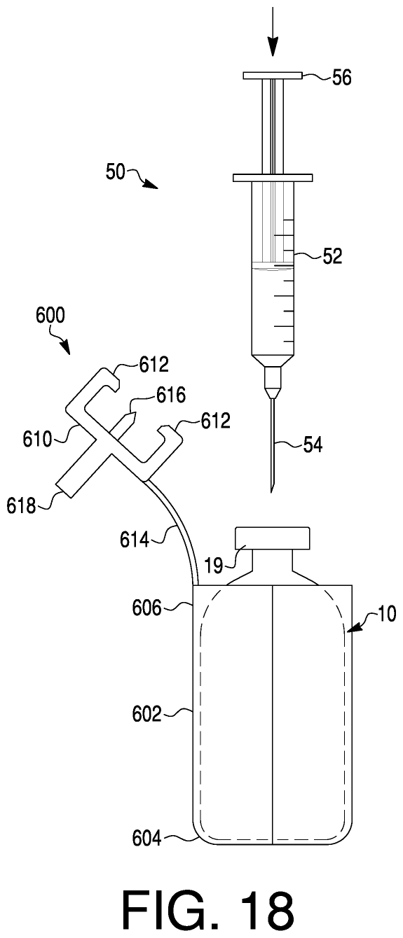

Alternatively, cap 610 may be decoupled from the mouth 19 to allow an external device to extend into the vial 10 and access the contents stored therein via mouth 19. For example, as seen in FIG. 18, the external device may include a syringe 50 having a barrel 52, a needle 54 extending distally from the barrel 52, and a plunger extending proximally from the barrel 52. Needle 616 may be inserted through the opening in vial 10 to remove the contents of vial 10.

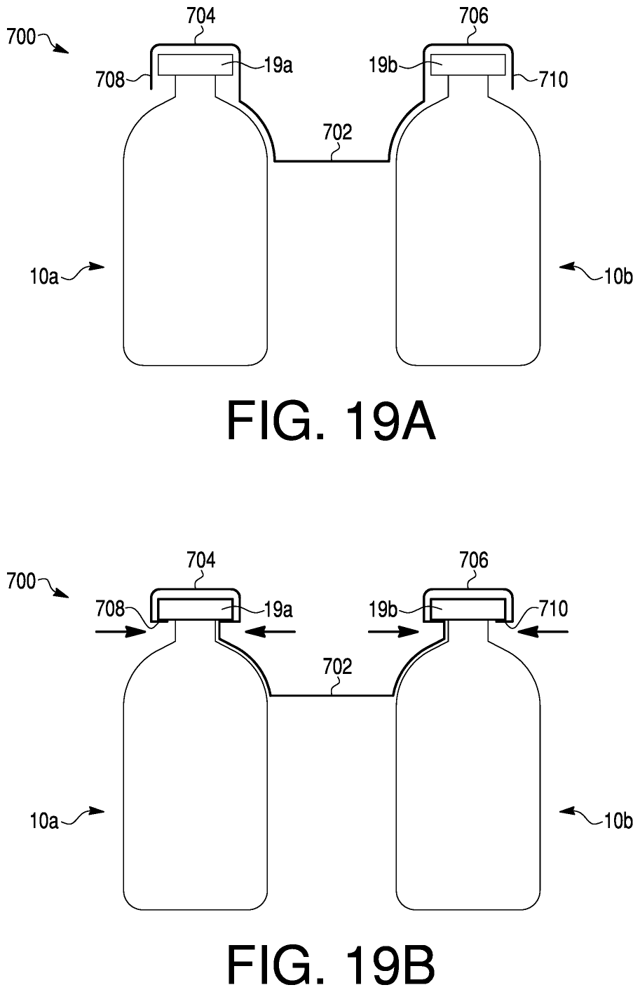

Referring now to FIG. 19A, an exemplary vial adapter assembly 700 is depicted. Vial adapter assembly 700 may be configured and operable to retain two or more vials together upon attaching portions of the vial adapter assembly 700 over each vial. Vial adapter assembly 700 may be crimped over the openings of multiple vials. Vial adapter assembly 700 may include a body 702 having a first outer portion 704 and a second outer portion 706 that is positioned opposite of the first outer portion 704. Body 702 may be formed of a flexible material, and configured to deform into a plurality of positions, shapes, and/or configurations between the first outer portion 704 and the second outer portion 706. For example, body 702 may be formed of aluminum or other various malleable metals. In one aspect, body 702 may be formed of a stamped piece of aluminum.

It should be appreciated that body 702 may have various suitable lengths defined between the first outer portion 704 and the second outer portion 706, such that body 702 may include one or more intermediate portions between the outer portions 704, 706. Body 702 may be positioned over the pair of vials 10a, 10b, with first outer portion 704 aligned with the first vial 10a and second outer portion 706 aligned with the second vial 10b. An outer end 708 of first outer portion 704 may be positioned adjacent to the mouth 19a of the first vial 10a, and an outer end 710 of second outer portion 706 may be positioned adjacent to the mouth 19b of the second vial 10b.

Referring to FIG. 19B, body 702 may be deformed in accordance with a size, shape, and/or cross-sectional profile of the first vial 10a and the second vial 10b to securely couple vial adapter assembly 700 to the pair of vials 10a, 10b. In particular, first outer portion 704 may be molded about the mouth 19a, such as the one or more surfaces and/or edges defining the mouth 19a, to securely engage the first vial 10a. Stated differently, first outer portion 704 may be crimped onto the mouth 19a. Outer end 708 of first outer portion 704 may be crimped so that it engages an undersurface of mouth 19a to retain vial adapter 700 on first vial 10a.

Second outer portion 706 may be molded about the mouth 19b, such as the one or more surfaces and/or edges defining the mouth 19b, to securely engage the second vial 10b. Outer end 708 may be conformed to and engaged against a surface and/or edge of the shoulder 19a to securely couple first outer portion 704 to the first vial 10a, and outer end 710 may be conformed to and engaged against a surface and/or edge of the mouth 19b to securely couple second outer portion 706 to the second vial 10b. Stated differently, second outer portion 706 may be crimped onto the mouth 19b. Outer end 710 of second outer portion 706 may be crimped so that it engages an undersurface of mouth 19b to retain vial adapter assembly 700 on second vial 10b. To crimp vial adapter assembly 700 to onto the opening portions of first and second vials 10a, 10b, body 702 may be deformed in response to an application of force thereto, such as, for example, manually by a user's hand or a tool, stamped by an automated machinery, and more.

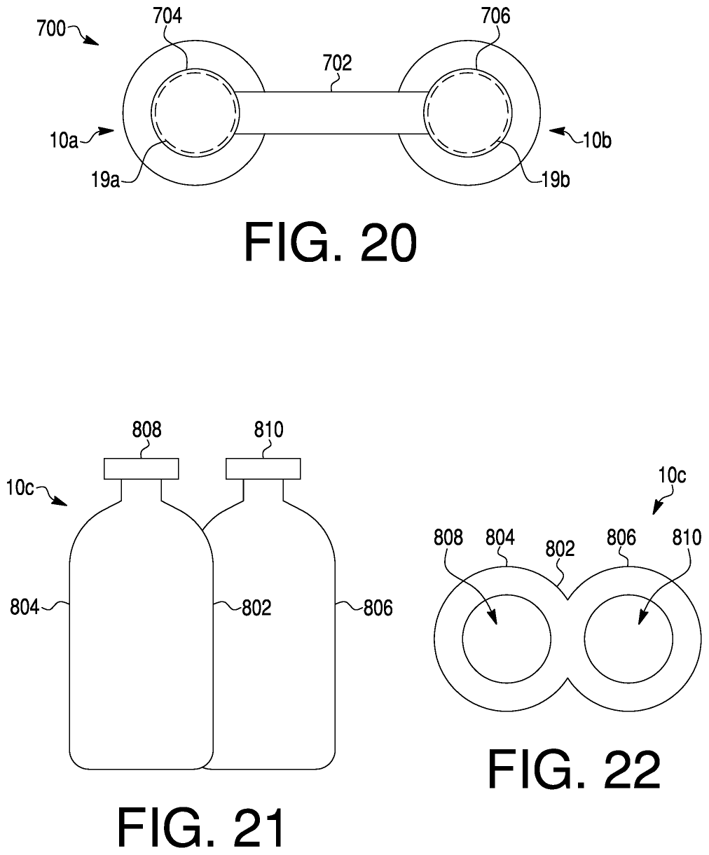

As seen in FIG. 20, vial adapter assembly 700 may be configured to enclose only a portion of the pair of vials 10a, 10b, such that a remaining portion of the first vial 10a and the second vial 10b may be uncovered by body 702. In other embodiments, vial adapter assembly 700 may be configured to enclose additional and/or fewer portions of the first vial 10a and/or the second vial 10b, such as the proximal ends, the bodies, and the distal ends, etc.

The embodiment of FIG. 21 depicts an alternative to the use of a separate vial adapter assembly. Rather than using a vial adapter into which multiple vials may be inserted and retained together, the embodiment of FIG. 21 depicts a double vial 10c that is formed together as a single, monolithic unit. For example, double vial 10c may include two vial portions that share a common wall and cannot be separated from one another. FIG. 22 depicts a cross-sectional view through a central region of double vial 10c showing portions 804, 806 of body 802 having a shared wall. Double vial 10c may be formed of molded plastic or glass, for example.

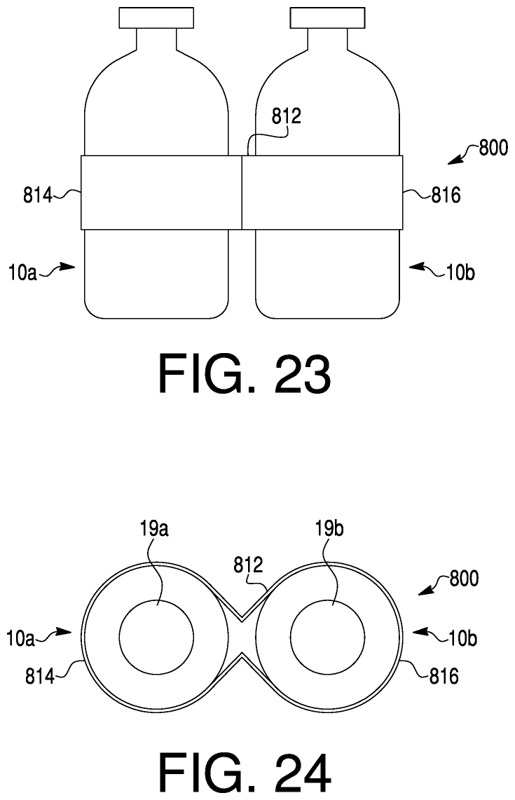

Referring now to FIGS. 23 and 24, rather than specially forming a double vial container as a single unit, vial adapter assembly 800 may function to retain two separate vials together in a similar manner. Vial adapter assembly 800 may include a body 812 having a first portion 814 and a second portion 816 that is positioned opposite of the first portion 814. First portion 814 may be sized, shaped, and configured to receive at least a portion of the first vial 10a, e.g., a central region of first vial 10a. Second portion 816 may be sized, shaped, and configured to receive at least a portion of the second vial 10b, e.g., a central region of second vial 10b.

Body 812 may be formed of a rigid material (e.g., plastic or metal), and, in some aspects, may be molded over the first vial 10a and the second vial 10b. Further, body 812 may include a single, moldable body. For example, body 812 may be formed of glass, plastic, metal, and/or various other materials. Although body 812 is depicted and described as having two portions 814, 816 for receiving the first vial 10a and the second vial 10b, respectively, body 812 may have additional portions for receiving more than two vials.

As best seen in FIG. 24, body 812 may be molded in accordance with a size, shape, and/or cross-sectional profile of the first vial 10a and the second vial 10b to securely couple vial adapter assembly 800 to the pair of first and second vials 10a, 10b. In particular, first portion 814 may be molded about a central region of first vial 10a to securely engage the first vial 10a. Second portion 816 may be molded about a central region of second vial 10b to securely engage the second vial 10b. In some aspects, an adhesive layer or glue may be used to help retain first and second vials 10a, 10b within first and second portions 814, 816. In the example, vial adapter assembly 800 may be configured to enclose only a portion of the pair of vials 10a, 10b. In other embodiments, vial adapter assembly 800 may be configured to enclose additional and/or fewer portions of the first vial 10a and/or the second vial 10b, such as the proximal ends, the bodies, and the distal ends, etc.

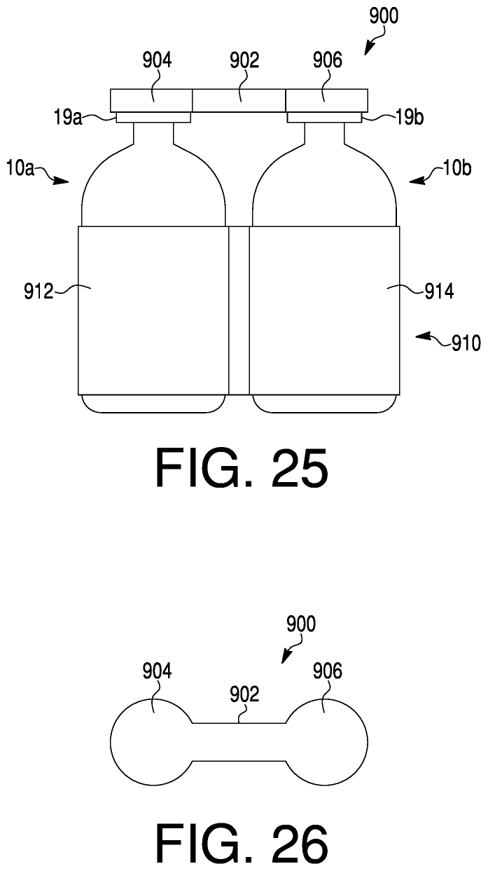

Referring now to FIG. 25, an exemplary vial adapter assembly 900 is depicted. Vial adapter assembly 900 may be configured and operable to retain and facilitate access (fluid communication) to two or more vials fixed together upon attaching portions of the vials to the vial adapter assembly 900. Vial adapter assembly 900 may include a cap 902 and a sleeve 910. Each of cap 902 and sleeve 910 may be configured to couple a plurality of vials to one another along respective portions of the vials. In the example, cap 902 and sleeve 910 may be cooperatively configured to couple the first vial 10a to the second vial 10b, however, in other embodiments, vial adapter assembly 900 may couple additional vials together. As seen in FIG. 26, cap 902 may include a first lid cover 904 and a second lid cover 906 that are integral with one another and that form a unitary body. First lid cover 904 may be sized, shaped, and configured to receive at least a portion of the first vial 10a, such as the mouth 19a. Second lid cover 906 may be sized, shaped, and configured to receive at least a portion of the second vial 10b, such as the mouth 19b. Cap 902 may be configured to couple the first vial 10a to the second vial 10b, and simultaneously expose or enclose openings of the first vial 10a and the second vial 10b with first lid cover 904 and second lid cover 906, respectively. In the example, cap 902 may be formed of any suitable material, such as, for example, plastic or metal.

Cap 902 may be a double flip-off cap configured to simultaneously disengage the first vial 10a and the second vial 10b in response to applying a proximal (pulling or pushing) force onto cap 902 relative to the vials 10a, 10b, thereby decoupling first lid cover 904 from the mouth 19a and second lid cover 906 from the mouth 19b. Accordingly, vial adapter assembly 900 may be configured to simultaneously inhibit fluid communication to the pair of vials 10a, 10b, such as from an external device, when cap 902 is coupled to the first vial 10a and the second vial 10b. Vial adapter assembly 900 may be further configured to simultaneously allow fluid communication to the pair of vials 10a, 10b in response to decoupling cap 902 from the first vial 10a and the second vial 10b.

Cap 902 may be used in conjunction with sleeve 910. Sleeve 910 may share some similarities with body 812 described above. Sleeve 910 may be over-molded around first and second vials 10a, 10b, or an adhesive layer (e.g., pressure sensitive adhesive) or glue may be used to retain first and second vials 10a, 10b within sleeve 910. Sleeve 910 may be a single unit or may be formed of more than one piece. For example, sleeve 910 may consist of two halves that fit together to surround first and second vials 10a, 10b. Forming sleeve 910 out of two or more pieces may aid in fitting sleeve 910 onto first and second vials 10a, 10b if an adhesive or glue is used. In other aspects, sleeve 910 may retain first and second vials, 10a, 10b using an adhesive or glue and may not surround a complete circumference of the first and second vials, as will be described further below. Sleeve 910 may be formed of any suitable material, for example, a plastic or metal.

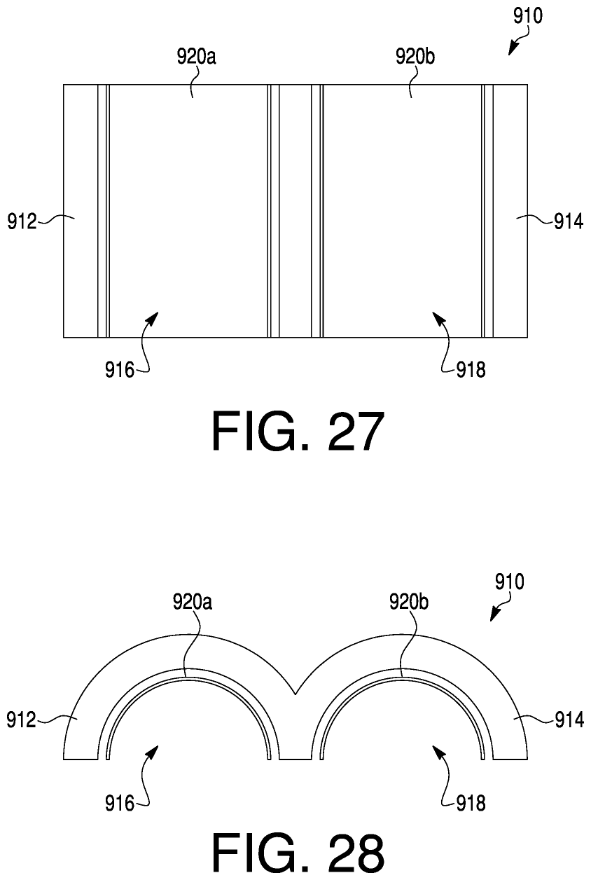

Referring to FIGS. 27-28, sleeve 910 may include a first portion 912 and a second portion 914 that is positioned opposite of the first portion 912. First portion 912 may define a first receptacle 920a that is sized, shaped, and configured to receive at least a portion of the first vial 10a, and second portion 914 may define a second receptacle 920b that is sized, shaped, and configured to receive at least a portion of the second vial 10b (see FIG. 25). In the example, first portion 912 may be configured to receive a portion of the body of the first vial 10a, and second portion 914 may be configured to receive a portion of the body of the second vial 10b.

As best seen in FIG. 28, first portion 912 and second portion 914 may each have a cross-sectional profile that conforms to an exterior profile of the bodies of vials 10a, 10b, respectively. In the example, first portion 912 may have a semi-circular shape such that sleeve 910 is configured to enclose a portion of the body when the first vial 10a is received within first receptacle 220a. Further, second portion 914 may have a semi-circular shape such that sleeve 910 is configured to enclose a portion of the body when the second vial 10b is received within second receptacle 920b. In other embodiments, first portion 912 and/or second portion 914 may have various other suitable cross-sectional profiles than that shown and described herein. In further embodiments, first portion 912 and second portion 914 may have different cross-sectional profiles relative to another.

Sleeve 910 may include a first engagement element 916 within first receptacle 920a for securing the first vial 10a to first portion 912, and a second engagement element 918 within second receptacle 920b for securing the second vial 10b to second portion 914. In the example, one or more of first engagement element 916 and/or second engagement element 918 may include an adhesive liner (e.g., pressure-sensitive adhesive, glue, etc.) and/or other suitable bonding materials for securing a vial to sleeve 910. Sleeve 910 may be configured to store each of the first vial 10a and the second vial 10b within the respective receptacle 916, 918 independent of the other. Further, sleeve 910 may be configured to maintain the first vial 10a and the second vial 10b in connection with one another after cap 902 is decoupled from the corresponding mouths 19a, 19b. Further, sleeve 910 depicted in FIG. 28 may constitute the entire sleeve, which may enclose approximately half of a circumference of each of first and second vials 10a, 10b. Alternatively, sleeve 910 shown in FIG. 28 may be half of a sleeve 910 and may be used in conjunction with a second half (not shown) to more fully enclose a circumference of the first and second vials 10a, 10b.

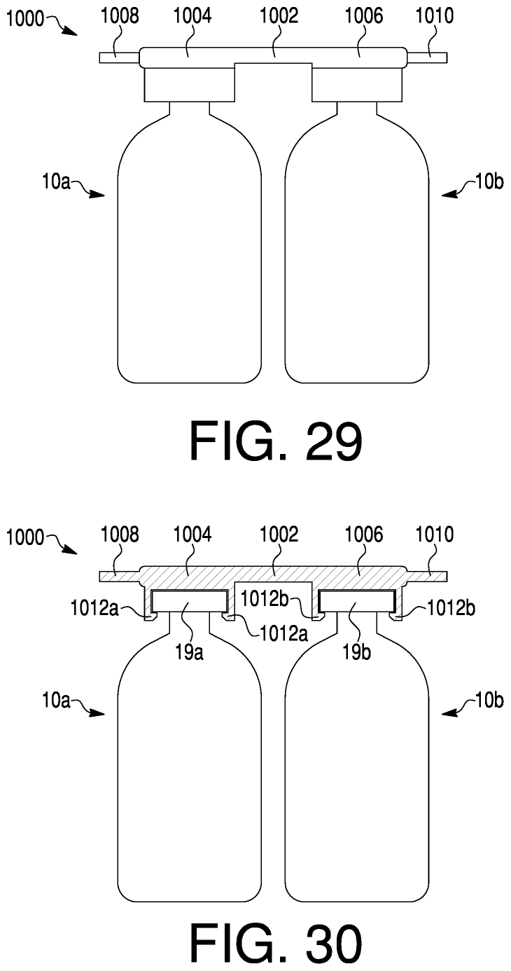

Referring now to FIG. 29, an exemplary vial adapter assembly 1000 is depicted. Vial adapter assembly 1000 may be configured and operable to retain and facilitate access (fluid communication) to two or more vials fixed together from the attachment of respective lid covers over each vial. Vial adapter assembly 1000 may include a tear-off, double flip-off cap. Vial adapter assembly 1000 may include a body 1002 configured to couple a plurality of vials to one another. In the example, body 1002 may be configured to couple the first vial 10a to the second vial 10b, however, in other embodiments, vial adapter assembly 1000 may couple additional vials together. Body 1002 may include a first lid cover 1004 and a second lid cover 1006. First lid cover 1004 may be sized, shaped, and configured to receive at least a portion of the first vial 10a, such as the mouth 19a. Second lid cover 1006 may be sized, shaped, and configured to receive at least a portion of the second vial 10b, such as the mouth 19b. Body 1002 may be configured to couple the first vial 10a to the second vial 10b, and simultaneously enclose or expose openings of the first vial 10a and the second vial 10b with first lid cover 1004 and second lid cover 1006, respectively. Body 1002, and first and second lid covers 1004, 1006 may be formed of two halves that extend parallel to one another. One half of first and second lid covers 1004, 1006 may be configured to engage a first side of the mouths 19a, 19b of first and second vials 10a, 10b, and the other half of first and second lid covers 1004, 1006 may be configured to engage a second side of the mouths of first and second vials 10a, 10b.

Vial adapter assembly 1000 may further include at least one of a first pull tab 1008 extending laterally outward from first lid cover 1004 and a second pull tab 1010 extending laterally outward from second lid cover 1006. Each of first pull tab 1008 and second pull tab 1010 may be sized, shaped, and configured to define a handle for manually manipulating body 1002, such as for deforming one or more of first lid cover 1004 and/or second lid cover 1006. As described further herein, vial adapter assembly 1000 may be configured to transition from a first (assembled) state to a second (disassembled) state in response to actuating one or more of first pull tab 1008 and/or second pull tab 1010.

As shown in FIG. 30, vial adapter assembly 1000 may include a pair of first clips 1012a and a pair of second clips 1012b for securing each of the first vial 10a and the second vial 10b to body 1002, respectively. In the example, the pair of first clips 1012a may extend distally (e.g., downward) from first lid cover 1004, and the pair of second clips 1012b may extend distally from second lid cover 1006. The pair of first clips 1012a may be at least partially flexible and configured to form a snap-fit connection with the mouth 19a of the first vial 10a received within first lid cover 1004. The pair of second clips 1012b may be at least partially flexible and configured to form a snap-fit connection with the mouth 19b of the second vial 10b received within second lid cover 1006. In other embodiments, vial adapter assembly 1000 may include various other engagement mechanisms for selectively coupling the first vial 10a and the second vial 10b to body 1002 without departing from a scope of this disclosure.

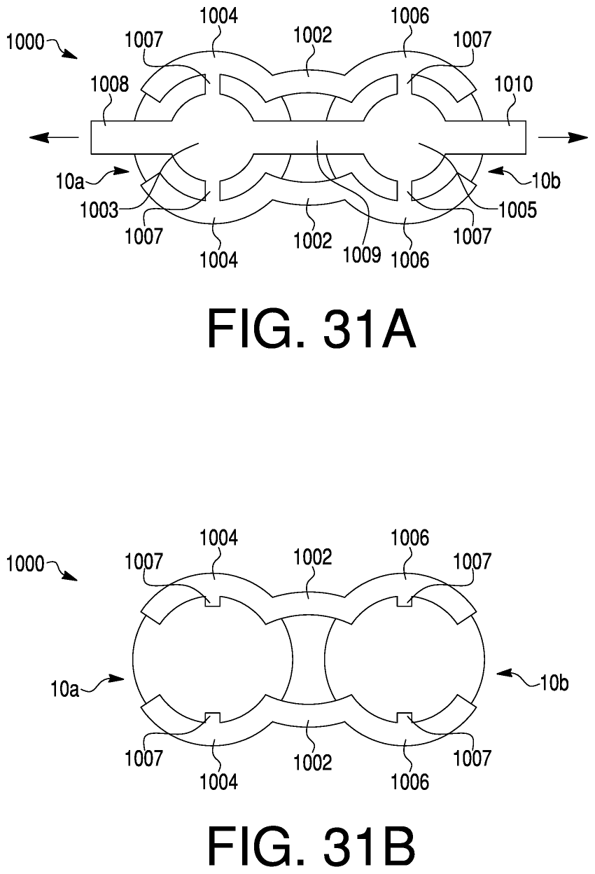

Referring to FIG. 31A, vial adapter assembly 1000 is shown in the first (assembled) configuration with body 1002 in a fully assembled state. In this instance, a first center portion 1003 of first lid cover 1004 may be positioned over and coupled to the mouth 19a of the first vial 10a, thereby enclosing an opening of the first vial 10a and coupling the first vial 10a to body 1002. A second center portion 1005 of second lid cover 1006 may be positioned over and coupled to the mouth 19b of the second vial 10b, thereby enclosing an opening of the second vial 10b and coupling the second vial 10b to body 1002.

In the example, first center portion 1003 may be coupled to first pull tab 1008, and first lid cover 1004 may be coupled to first center portion 1003 by one or more frangible branches 1007. Second center portion 1005 may be coupled to second pull tab 1010, and f second lid cover 1006 may be coupled to second center portion 1005 by one or more frangible braches 1007. As described further herein, frangible branches 1007 may be at least partially deformable to separate each of first center portion 1003 from first lid cover 1004, and second center portion 1005 from second lid cover 1006, when transitioning vial adapter assembly 1000 to the second (disassembled) state.

Still referring to FIG. 31A, first pull tab 1008 may be coupled to second pull tab 1010 by an intermediate strip 1009 positioned between first pull tab 1008 and second pull tab 1010. In some embodiments, vial adapter assembly 1000 may be configured such that actuation of first pull tab 1008 is independent from an actuation of second pull tab 1010, and vice versa. Stated differently, applying a force (e.g., either a pulling or peeling force) onto either first pull tab 1008 and/or second pull tab 1010 may not cause a corresponding movement of the opposing tab across intermediate strip 1009. In this instance, intermediate strip 1009 may be configured to deform upon actuation of either first pull tab 1008 and/or second pull tab 1010, such that body 1002 may sever along intermediate strip 1009.

In other embodiments, vial adapter assembly 1000 may be configured such that actuation of first pull tab 1008 may provide for a corresponding actuation of second pull tab 1010 via intermediate strip 1009, and vice versa. In this instance, applying a force (e.g., either a pulling or peeling force) onto either first pull tab 1008 and/or second pull tab 1010 may cause a corresponding movement of the opposing tab and intermediate strip 1009. In the example, body 1002 may be formed of a flexible material, such as, for example, plastic.

As seen in FIG. 31B, body 1002 may be at least partially deformable in response to an application of force, such as at first pull tab 1008 and/or second pull tab 1010. In the example, at least a portion of first lid cover 1004, particularly first center portion 1003, may be removable from a remaining portion of first lid cover 1004 (e.g., a periphery edge) in response to pulling first pull tab 1008. In particular, the one or more frangible branches 1007 may be severed upon actuating first pull tab 1008, causing first center portion 1003 to be removed and exposing the mouth 19a (e.g., a stopper in mouth 19a) of the first vial 10a.

At least a portion of second lid cover 1006, particularly second center portion 1005, may be removable from a remaining portion of second lid cover 1006 (e.g., a periphery edge) in response to pulling second pull tab 1010. The one or more frangible branches 1007 may be severed upon actuating second pull tab 1010, causing second center portion 1005 to be removed and exposing the mouth 19b (e.g., a stopper in mouth 19a) of the second vial 10b. An opening of the first vial 10a and the second vial 10b may be uncovered upon independently actuating each of first pull tab 1008 and second pull tab 1010, respectively, to selectively remove each of the center portions 1003, 1005. Alternatively, however, only one pull tab may be included, and a force may be applied to the single pull tab to remove first and second center portions 1003, 1005, and intermediate strip 1009 all at once by rupturing the frangible branches 1007.

As shown in FIG. 31B, body 1002 may be configured to maintain engagement with the mouths 19a, 19b upon removal of first center portion 1003 and second center portion 1005. In other words, vial adapter assembly 1000 may maintain the first vial 10a in connection to the second vial 10b when in the second (disassembled) state. In the example, with at least a portion of first lid cover 1004 maintained over the first vial 10a and a portion of second lid cover 1006 maintained over the second vial 10b, vial adapter assembly 1000 may be configured to inhibit an ancillary cap from covering the first vial 10a and/or the second vial 10b. As such, vial adapter assembly 1000 may prevent re-use of the first vial 10a and the second vial 10b upon facilitating access to the contents of the vials. In other words, the remaining portions of first lid cover 1004 and second lid cover 1006 may provide a tamper proof indicator for inhibiting re-use.

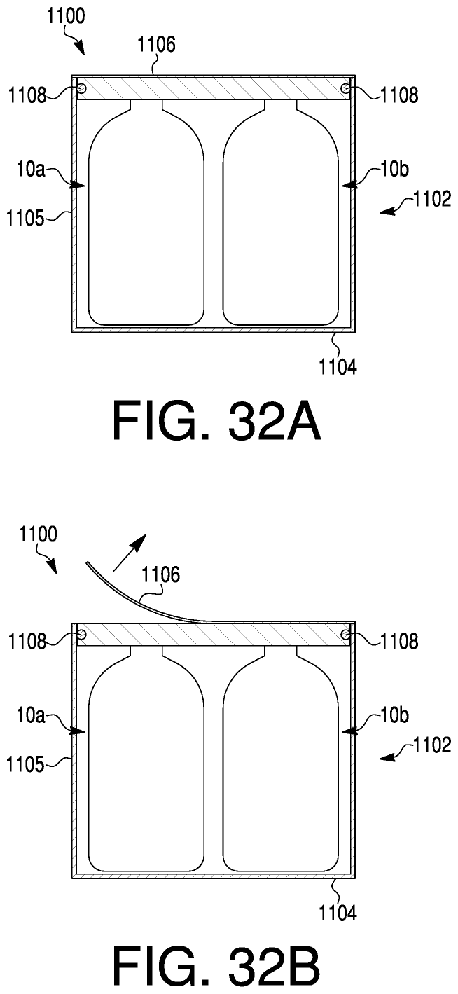

Referring now to FIG. 32A, an exemplary vial adapter assembly 1100 is depicted. Vial adapter assembly 1100 may be configured and operable to retain and facilitate simultaneous access (fluid communication) to two or more vials housed together within a blister pack that is sealed by a blister pack seal. Vial adapter assembly 1100 may include a housing 1102 configured to couple a plurality of vials to one another. In the example, housing 1102 may be configured to couple the first vial 10a to the second vial 10b, however, in other embodiments, vial adapter assembly 1100 may couple additional vials together. Housing 1102 may include a fixed bottom wall 1104, at least one sidewall 1105, and a removable top wall 1106 that collectively define an enclosure for containing the first vial 10a and the second vial 10b. In the example, housing 1102 may be a blister pack, and removable top wall 1106 may be a blister pack seal that is configured to move in response to applying a proximal (e.g., pulling or peeling) force thereto.

Housing 1102 may be sized and shaped to fully enclose the first vial 10a and the second vial 10b between fixed bottom wall 1104 and removable top wall 1106. Vial adapter assembly 1100 may include a seal 1108 extending about a perimeter of housing 1102 adjacent to removable top wall 1106. Seal 1108 may be configured to inhibit fluid communication of the first vial 10a and the second vial 10b, and particularly the contents stored therein, with a space outside of housing 1102 to maintain sterility of said contents. Seal 1108 may be broken upon removal of removable top wall 1106. In some embodiments, vial adapter assembly 1100 may include separate receptacles for each of the first vial 10a and the second vial 10b within housing 1102 to inhibit fluid communication between the first vial 10a and the second vial 10b. Removable top wall 1106 may be configured to simultaneously enclose or expose openings of the first vial 10a and the second vial 10b when vial adapter assembly 1100 is in a first (closed) configuration. Alternatively, blister packaging housing 1102 may contain the first vial 10a and the second vial 10b, which may need to be individually opened once the removable top wall 1106 is at least partially removed.

As seen in FIG. 32B, vial adapter assembly 1100 may be transitioned from a first (closed) configuration to a second (open) configuration upon removal of removable top wall 1106. In the example, removable top wall 1106 may be configured to move relative to the fixed bottom wall 1104 and/or the at least one sidewall 1105 to facilitate access into housing 1102. In this instance, openings of the first vial 10a and the second vial 10b may be simultaneously uncovered by removable top wall 1106, thereby facilitating access to the contents stored therein. In other embodiments, each of the receptacles defined within housing 1102 may include a separate removable top wall. Vial adapter assembly 1100 is configured to inhibit reassembly of removable top wall 1106 upon removal of removable top wall 1106. Thus, vial adapter assembly 1100 may be configured to prevent re-use of the first vial 10a and the second vial 10b upon facilitating access to the contents of the vials. In other words, removable top wall 1106 may provide a tamper proof indicator for inhibiting re-use.

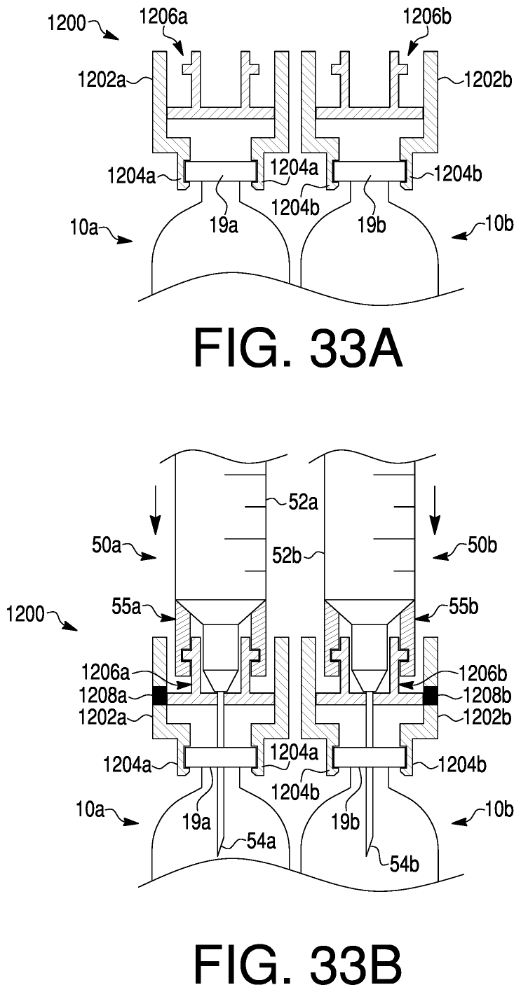

Referring now to FIG. 33A, an exemplary vial adapter system 1200 is depicted. Vial adapter system 1200 may be configured and operable to facilitate access (fluid communication) to a vial through a corresponding connector capable of coupling an external device (e.g., a syringe) thereto. Vial adapter system 1200 may include at least one adapter for each of a plurality of vials. In the example, vial adapter system 1200 may include at least a first adapter 1202a configured to couple to the mouth 19a of the first vial 10a, and at least a second adapter 1202b configured to couple to the mouth 19b of the second vial 10b. First adapter 1202a and second adapter 1202b are independent of one another, such that vial adapter system 1200 permits individual access to each of the first vial 10a and the second vial 10b. As described in detail herein, each of the first vial 10a and the second vial 10b may include a respective visual indicator (e.g., a colored label) that corresponds to a matching visual indicator (e.g., color) on first adapter 1202a and second adapter 1202b, respectively.

First adapter 1202a may include a pair of first clips 1204a at a distal (bottom) end and a first connector 1206a at a proximal (top) end. The pair of first clips 1204a may be configured to engage the mouth 19a, thereby coupling first adapter 1202a to the first vial 10a. Second adapter 1202b may include a pair of second clips 1204b at a distal (bottom) end and a second connector 1206b at a proximal (top) end. The pair of second clips 1204b may be configured to engage the mouth 19b, thereby coupling second adapter 1202b to the second vial 10b.

As seen in FIG. 33B, first connector 1206a may be configured to couple first adapter 1202a to an external device, such as, for example, a first syringe 50a having a barrel 52a, a needle 54a, and a locking mechanism 55a. In the example, first connector 1206a may include a luer lock that is configured to mate with locking mechanism 55a of the first syringe 50a. First adapter 1202a may further include a first actuator 1208a that is configured to selectively establish fluid communication between the first vial 10a and the first syringe 50a. In particular, actuation of first actuator 1208a may fluidly couple the first syringe 50a to the first vial 10a by translating needle 54a of the first syringe 10a into the first vial 10a. In some embodiments, first actuator 1208a may include a push-button having a visual indicator (e.g., a color) that matches a corresponding color on the first vial 10a indicative of the content stored in the first vial 10a.

Second connector 1206b may be configured to couple second adapter 1202b to an external device, such as, for example, a second syringe 50b having a barrel 52b, a needle 54b, and a locking mechanism 55b. In the example, second connector 1206b may include a luer lock that is configured to mate with locking mechanism 55b of the second syringe 50b. Second adapter 1202b may further include a second actuator 1208b that is configured to selectively establish fluid communication between the second vial 10b and the second syringe 50b. In particular, actuation of second actuator 1208b may fluidly couple the second syringe 50b to the second vial 10b by translating needle 54b of the second syringe 10b into the second vial 10b. In some embodiments, second actuator 1208b may include a push-button having a visual indicator (e.g., a color) that matches a corresponding color on the second vial 10b indicative of the content stored in the second vial 10b. It should be appreciated that the color of second actuator 1208b is different from the color of first actuator 1208a to facilitate an identification of the corresponding adapter to be connected to the respective vial.

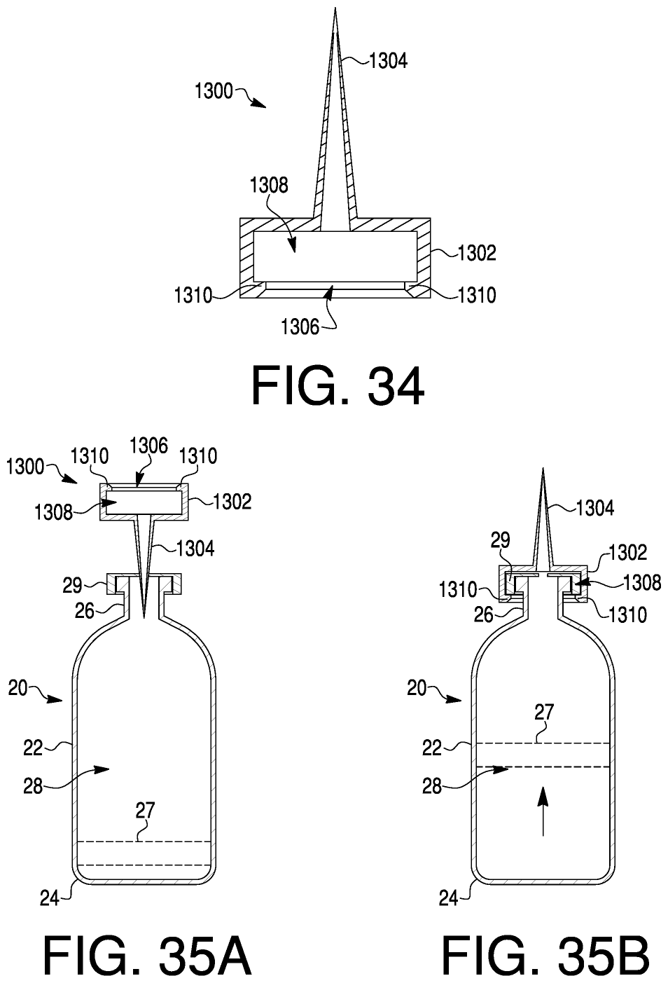

Referring now to FIG. 34, an exemplary adapter 1300 is depicted. Adapter 1300 may be configured and operable to facilitate access (fluid communication) and delivery of a content stored in a vial using respective ends of the vial adapter assembly 1300. Adapter 1300 may be configured to open and facilitate delivery of a fluid substance stored in a vial or cartridge, such as cartridge 20 shown in FIGS. 35A-35B. It should be appreciated that at least one adapter 1300 may be coupled to or come separate with each of a plurality of cartridges 20. Adapter 1300 may include a housing 1302 having a needle 1304 at a first end and an opening 1306 at a second end that is opposite of the first end. Housing 1302 may define a receptacle 1308 for receiving at least a portion of cartridge 20 to couple adapter 1300 to cartridge 20 (see FIG. 35B).

Housing 1302 may include a pair of clips 1310 positioned adjacent to the opening 1306 at the second end. The pair of clips 1310 may be configured to engage a portion of cartridge 20 for securely coupling adapter 1300 to the cartridge 20 (see FIG. 35B). While clips 1310 are shown, it should be understood that adapter 1300 may attach to cartridge 20 in any suitable manner, for example, snap fit, screw on, friction fit, etc. Needle 1304 may include a hollow channel that is in fluid communication with the receptacle 1308 such that a fluid received within adapter 1300 at the receptacle 1308 (e.g., via the opening 1306) may be communicated through the hollow channel and to needle 1304.

In exemplary use, as seen in FIG. 35A, adapter 1300 may be positioned and/or oriented in a first configuration to facilitate access into a body 22 of cartridge 20. For example, adapter 1300 may be positioned to align needle 1304 with a mouth 29 of cartridge 20. Needle 1304 may be configured to pierce a seal of cartridge 20 located at the mouth 29 to facilitate access into a cavity 28 of body 22. Upon piercing the seal of cartridge 20, adapter 1300 may be transitioned from the first configuration to a second configuration by reorienting adapter 1300 to position opening 1306 in alignment with the opening formed at the mouth 29a.

As seen in FIG. 35B, adapter 1300 may be coupled to the cartridge 20 by attaching housing 1302 to the shoulder 29 when adapter 1300 is in the second configuration. Particularly, the pair of clips 1310 may be configured to engage an edge of the mouth 29 to securely couple housing 1302 to the cartridge 20. The opening 1306 and/or the receptacle 1308 may be sized, shaped, and configured to receive at least a portion of the cartridge 20, such as, for example, the mouth 29. In this instance, adapter 1300 may be in fluid communication with the cartridge 20 such that the content stored in the cavity 28 may be received through the opening 1306, the receptacle 1308, and into the hollow channel of needle 1304.

For example, a plunger 27 of the cartridge 20 disposed within the body 22 may be actuated to expel the content stored in the cavity 28 into housing 1302. Needle 1304 may be positioned at a target treatment site to deliver the content from the cartridge 20 to the target treatment site. Accordingly, needle 1304 may be configured to facilitate access to the cartridge 20 when adapter 1300 is in the first configuration, and to deliver the content stored in the cartridge 20 when adapter 1300 is in the second configuration.

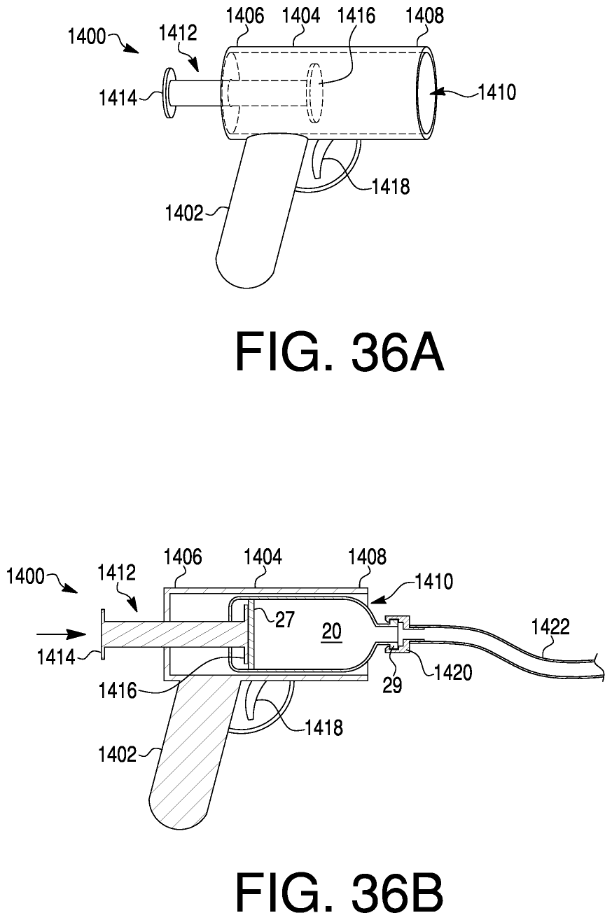

Referring now to FIG. 36A, an exemplary adapter 1400 is depicted. Adapter 1400 may be configured and operable to retain and facilitate independent delivery of content stored within two or more cartridges housed together within separate barrels via respective plungers positioned within each barrel. Adapter 1400 may include a handle 1402, a pair of barrels 1404 each having a proximal end 1406 and a distal end 1408, and at least one plunger 1412 movably coupled to each of the pair of barrels 1404. In the example, adapter 1400 may include at least two barrels 1404 positioned adjacent to one another, and each barrel 1404 may be configured to receive at least one cartridge 20. In other embodiments, adapter 1400 may be include additional barrels 1404 for receiving additional cartridges 20. Although a single barrel 1404 and cartridge 20 is shown and described herein with respect to adapter 1400, it should be understood that adapter 1400 may include at least a pair of barrels 1404 for receiving at least a pair of cartridges 20. Accordingly, the following description may be applicable to each cartridge 20 received within adapter 1400.

Plunger 1412 may extend into barrel 1404 through proximal end 1406. For example, plunger 1412 may include a proximal end 1414 positioned outside of barrel 1404 and proximal to proximal end 1406, and a distal end 1416 positioned inside barrel 1404 and distal to proximal end 1406. Adapter 1400 may further include an actuator 1418 coupled to plunger 1412, and configured to move plunger 1412 relative to barrel 1404 in response to an actuation of actuator 1418.

Still referring to FIG. 36A, adapter 1400 may include an opening 1410 at distal end 1408 that is configured to receive the cartridge 20. Barrel 1404 may be sized and shaped to at least partially receive the cartridge 20 within adapter 1400 via opening 1410. Barrel 1404 may be configured to securely engage the cartridge 20, thereby fixing an axial position of cartridge 20 relative to proximal end 1406 and/or distal end 1408. When disposed within barrel 1404, the distal end 1416 of plunger 1412 may be received within the cartridge 20 and positioned against the plunger 27 of the cartridge 20. Alternatively, plunger 27 or the cartridge 20 may form a proximal (bottom) surface of cartridge 20 and may be moveable towards the mouth of cartridge 20 upon the application of force. In such an embodiment, plunger 1412 may be aligned with the bottom of cartridge 20 (i.e., the plunger 27). Accordingly, plunger 1412 is configured to move the plunger 27 when the cartridge 20 is received within barrel 1404.

As seen in FIG. 36B, the cartridge 20 may be disposed within barrel 1404 with a proximal (bottom) end of the cartridge 20 positioned adjacent to the proximal end 1406 and the mouth 29 positioned adjacent to opening 1410. The distal end 1416 of plunger 1412 may be received within or in contact with a portion of the cartridge 20, and may abut against the plunger 27 of the cartridge 20. The mouth 29 may be coupled to a flexible tube 1422 to facilitate fluid communication between the cartridge 20 and a distal end of the flexible tube 1422 (not shown) positioned at, for example, a container (e.g., an intravenous (IV) bag). In exemplary use, actuator 1418 may be actuated to move plunger 1412 relative to barrel 1404 in a distal direction, thereby causing the distal end 1416 of plunger 1412 to abut against the plunger 27. In this instance, the content stored in the cartridge 20 may be expelled through the flexible tube 1422 and toward the container as the plunger 27 is urged distally within the cartridge 20.

In the example, actuation of actuator 1418 may provide for a simultaneous movement of each plunger 1412 disposed within the pair of barrels 1404, each of which may be coupled to at least one cartridge 20. Accordingly, actuator 1418 may be configured to simultaneously expel the contents of each cartridge 20 coupled to adapter 1400. In other embodiments, actuator 1418 may be configured to independently actuate each plunger 1412, thereby delivering the contents of each cartridge 20 to the container separately from one another.

In one embodiment, adapter 1400 may be configured such that actuator 1418 may be actuated to move plunger 1412 to a predetermined position for setting a predefined volume of the content to be expelled prior to delivery to the container. For example, prior to coupling the flexible tube 1422 to the mouth 29, actuator 1418 may be initially actuated to cause plunger 1412 to translate distally relative to barrel 1404 until stopping at the predetermined position that is indicative of the set volume. In this instance, the flexible tube 1422 may be coupled to the mouth 29 and actuator 1418 may be actuated to cause plunger 1412 to translate further for delivering the set volume of content to the container via the flexible tube 1422. In some aspects, prior to or after coupling the flexible tube 1422 to the mouth 29, adapter assembly 1400 may be set to stop plunger 1412 after having moved a set amount in a distal direction, which may correspond to a set amount of content being expelled from the container. Accordingly, vial adapter assembly 1400 may allow for controlling a dose of content being expelled from cartridge 20.

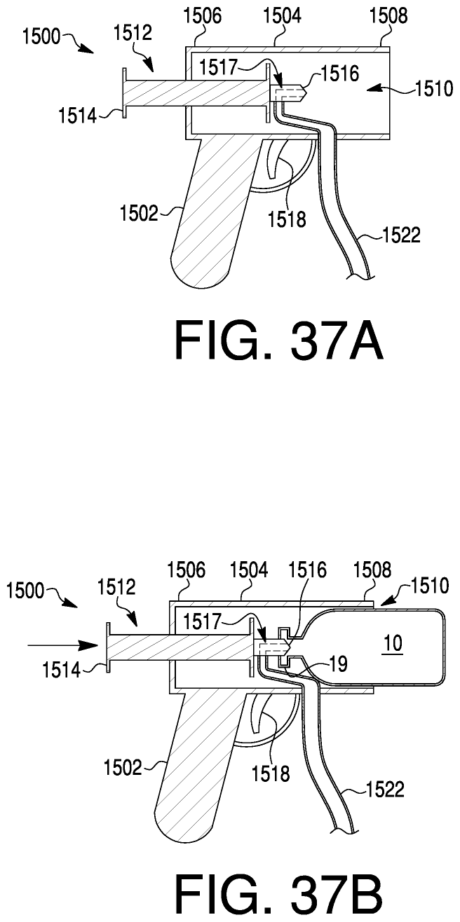

Referring now to FIG. 37A, an exemplary adapter 1500 is depicted. Adapter 1500 may be configured and operable to retain and facilitate independent delivery of content stored within two or more vials housed together within separate barrels via respective plungers positioned within each barrel. Adapter 1500 may include a handle 1502, a pair of barrels 1504 each having a proximal end 1506 and a distal end 1508, and at least one plunger 1512 movably coupled to each of the pair of barrels 1504. In the example, adapter 1500 may include at least two barrels 1504 positioned adjacent to one another, and each barrel 1504 may be configured to receive at least one vial 10. In other embodiments, adapter 1500 may include additional barrels 1504 for receiving additional vials 10. Although a single barrel 1504 and vial 10 are shown and described herein with respect to adapter 1500, it should be understood that adapter 1500 includes at least a pair of barrels 1504 for receiving at least a pair of vials 10. Accordingly, the following description may be applicable to each vial 10 received within adapter 1500.

Plunger 1512 may extend into barrel 1504 through proximal end 1506. For example, plunger 1512 may include a proximal end 1514 positioned outside of barrel 1504 and proximal to proximal end 1506, and a connector 1516 positioned inside barrel 1504 and distal to proximal end 1506. In some embodiments, connector 1516 may have a sharp distal tip. Adapter 1500 may further include an actuator 1518 coupled to plunger 1512, and configured to move plunger 1512 relative to barrel 1504 in response to an actuation of actuator 1518.

Still referring to FIG. 37A, adapter 1500 may further include an opening 1510 at distal end 1508 that is configured to receive the vial 10. Barrel 1504 may be sized and shaped to at least partially receive the vial 10 within adapter 1500 via opening 1510. Barrel 1504 may be configured to securely engage the vial 10, thereby fixing an axial position of vial 10 relative to proximal end 1506 and/or distal end 1508. Connector 1516 of plunger 1512 may include a channel 1517 that extends between a first open end positioned on the distal tip of connector 1516, and a second open end along a sidewall of connector 1516. The second open end of the channel 1517 may be fluidly coupled to a flexible tube 1522 to facilitate fluid communication between connector 1516 and a distal end of the flexible tube 1522 (not shown) positioned at, for example, a container (e.g., an IV bag). Connector 1516 may further include another channel, not shown, extending from a first open end positioned on the distal tip of the distal end 1516 longitudinally through the plunger 1512.

As seen in FIG. 37B, the vial 10 may be disposed within barrel 1504 with the mouth 19 positioned adjacent to connector 1516 of plunger 1512. When disposed within barrel 1504, connector 1516 of plunger 1512 may be received within the vial 10 through the mouth 19. In exemplary use, actuator 1518 may be actuated to move plunger 1512 relative to barrel 1504 in a distal direction, thereby pushing air present in plunger 1512 out of connector 1516 and into vial 10. This causes an increase in pressure within the vial 10, thereby causing the content stored within the vial 10 (e.g., a fluid substance) to exit the vial 10 from the mouth 19 via the channel 1517.

Thus, plunger 1512 is configured to compel the content outward from within the vial 10 as the plunger 1512 moves further distally towards connector 1516. As such, the content may be received through the first open end of the channel 1517 along connector 1516, through the channel 1517, and into the flexible tube 1522 coupled to the channel 1517 at the second open end. In the example, actuation of actuator 1518 may provide for a simultaneous movement of each plunger 1512 disposed within the pair of barrels 1504, each of which may be coupled to at least one vial 10. Accordingly, actuator 1518 may be configured to simultaneously expel the contents of each vial 10 coupled to adapter 1500. In other embodiments, actuator 1518 may be configured to independently actuate each plunger 1512, thereby delivering the contents of each vial 10 to the container separately from one another.

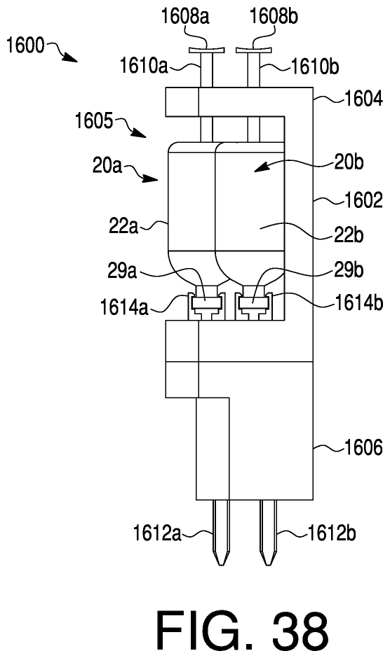

Referring now to FIG. 38, an exemplary adapter 1600 is depicted. Adapter 1600 may be configured and operable to retain and facilitate independent delivery of content stored within two or more cartridges positioned together via respective plungers coupled to each vial. Adapter 1600 may include a handle 1602 having a longitudinal length defined between a proximal end 1604 and a distal end 1606. Adapter 1600 may include a receptacle 1605 along the handle 1602 for receiving at least a pair of cartridges 20a, 20b. In the example, a first cartridge 20a and a second cartridge 20b is coupled to handle 1602 within the receptacle 1605. Receptacle 1605 may be at least partially exposed to an exterior surface of handle 1602 to facilitate a selective attachment of the cartridges 20a, 20b to handle 1602. Additionally, receptacle 1605 may allow for a visual inspection of a first body 22a of the first cartridge 20a and a second body 22b of the second cartridge 20b.

Handle 1602 may include a first pair of clips 1614a or other suitable connection mechanism within the receptacle 1605 for coupling the mouth 29a of the first cartridge 20a to handle 1602, and a second pair of clips 1614b or other suitable connection mechanism for coupling the mouth 29b of the second cartridge 20b to handle 1602. Actuator 1600 may further include a first plunger 1610a and a second plunger 1610b coupled to the first cartridge 20a and the second cartridge 20b, respectively. In the example, first plunger 1610a may be configured to extend into the first body 22a to urge the content stored in the first cartridge 20a outwardly upon actuation of first plunger 1610a. Second plunger 1610b may be configured to extend into the second body 22b to urge the content stored in the second cartridge 20b outwardly upon actuation of second plunger 1610b.

Still referring to FIG. 38, each of the plungers 1610a, 1610b may include an actuator 1608a, 1608b positioned adjacent to the proximal end 1604 of handle 1602 for selectively actuating plungers 1610a, 1610b, respectively. Accordingly, vial adapter 1600 may be configured such that the content stored in each of the cartridges 20a, 20b may be accessed independently of one another upon actuation of the corresponding actuator 1608a, 1608b. In the example, the first actuator 1608a and the second actuator 1608b may include a lever and/or a depressor, each of which may be configured to translate the first plunger 1610a and the second plunger 1610 through the corresponding cartridge 20a, 20b, respectively.

Adapter 1600 may further include a first pipette 1612a and a second pipette 1612b coupled to handle 1602 and extending distally from the distal end 1606. The first pipette 1612a may be in fluid communication with the first cartridge 20a, and the second pipette 1612b may be in fluid communication with the second cartridge 20b. In some embodiments, one or more of first pipette 1612a and/or second pipette 1612b may be connected to an external device, such as, for example, via a luer lock connection. In some aspects, adapter 1600 may allow for individually actuating cartridges 20a, 20b, and may allow a user to actuate either or both cartridges using one hand.

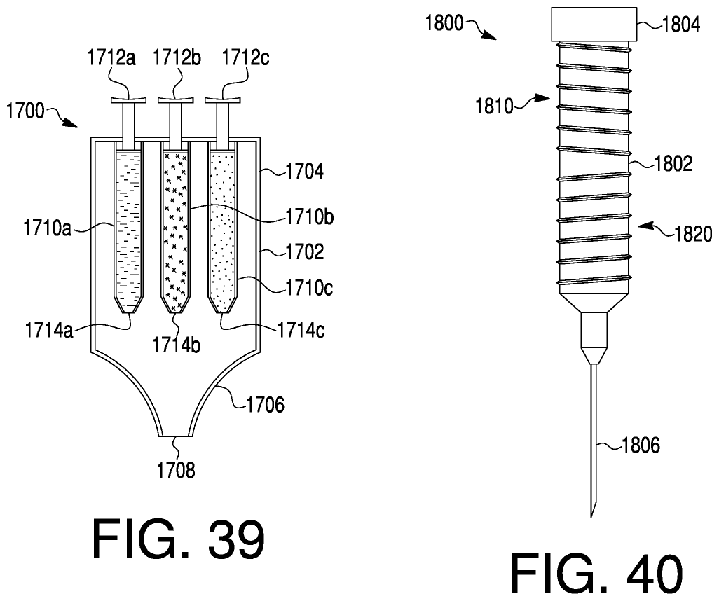

Referring now to FIG. 39, an exemplary adapter 1700 is depicted. Adapter 1700 may be configured and operable to retain and facilitate independent delivery of content stored within a plurality of cartridges housed together via respective actuators coupled to each cartridge. Adapter 1700 may include a housing 1702 having a longitudinal length defined between a proximal end 1704 and a distal end 1706. Distal end 1706 may terminate at a distal tip 1708 that includes an opening. As described further herein, a size, shape, and/or configuration of distal end 1706 may be configured to guide one or more cartridges disposed within housing 1702 toward distal tip 1708, such as a tapered shape, etc.

Adapter 1700 may include a plurality of cartridges disposed within housing 1702. In the example, adapter 1700 includes at least a first cartridge 1710a, a second cartridge 1710b, and a third cartridge 1710c. Each of the cartridges 1710a, 1710b, 1710c may store a different substance. First cartridge 1710a may include a proximal actuator 1712a extending outwardly from housing 1702, e.g., at the proximal end 1704, and a distal end 1714a having an opening for delivering a first substance stored within first cartridge 1710a. Second cartridge 1710b may include a proximal actuator 1712b extending outwardly from housing 1702, e.g., at the proximal end 1704, and a distal end 1714b having an opening for delivering a second substance stored within second cartridge 1710b. Third cartridge 1710c may include a proximal actuator 1712c extending outwardly from housing 1702, e.g., at the proximal end 1704, and a distal end 1714c having an opening for delivering a third substance stored within third cartridge 1710c.

Still referring to FIG. 39, adapter 1700 may be configured such that each of the actuators 1712a, 1712b, 1712c may be actuated independently relative to the others, such that a substance stored within each cartridge 1710a, 1710b, 1710c may be delivered from adapter 1700 separately from one another. In some embodiments, each of the actuators 1712a, 1712b, 1712c may be configured to require a first actuation to translate the corresponding distal end 1714a, 1714b, 1714c towards distal tip 1708 or outward from housing 1702 via the opening at distal tip 1708, and a second actuation for delivering the substance stored within the respective cartridge 1710a, 1710b, 1710c.

Accordingly, it should be appreciated that each of the cartridges 1710a, 1710b, 1710c may extend outwardly from housing 1702 via the same opening at distal tip 1708. In some embodiments, housing 1702 may be connected to an external device, such as, for example, via a luer lock connection at the distal tip 1708. Vial adapter assembly 1700 may allow for multiple fluid paths in a smaller footprint.

Referring now to FIG. 40, an exemplary syringe 1800 is depicted. Syringe 1800 may be configured and operable to retain and facilitate independent delivery of two or more volumes of a fluid substance stored within the syringe via movement (e.g., a rotation) of an actuator in distinct directions. Syringe 1800 may include a barrel 1802, an actuator 1804 coupled to barrel 1802 at a proximal end of barrel 1802, and a needle 1806 extending distally from a distal end of barrel 1802. Barrel 1802 may be configured to store a fluid substance (e.g., a medicament) within a cavity of barrel 1802. Actuator 1804 may include a plunger disposed within the cavity of barrel 1802 for expelling the fluid substance in response to movement of actuator 1804 relative to barrel 1802. Needle 1806 may be in fluid communication with the cavity of barrel 1802, and configured to deliver the fluid substance from barrel 1804 upon actuation of actuator 1804.

Actuator 1804 may be rotatably coupled to an exterior surface of barrel 1802. In some embodiments, actuator 1804 may include a rotatable knob and/or dial. Actuator 1804 may be configured to expel a first volume of the fluid substance stored in barrel 1802 when rotating in a first direction relative to barrel 1802, and expel a second volume of the fluid substance stored in barrel 1802 when rotating in a second direction relative to barrel 1802. Barrel 1804 may include a first threaded portion 1810 extending along a longitudinal length of barrel 1804 from a proximal portion to an intermediate portion of barrel 1804, and a second threaded portion 1820 extending along a longitudinal length of barrel 1804 from the intermediate portion to a distal portion of barrel 1804.

Still referring to FIG. 40, first threaded portion 1810 may be oriented in a first direction and second threaded portion 1820 may be oriented in a second direction that is different from the first direction. Actuator 1804 may include a threaded portion along an interior surface of actuator 1804, such that the threaded portion of actuator 1804 may be configured to mesh with first threaded portion 1810 and second threaded portion 1820 when actuator 1804 is coupled to an exterior surface of barrel 1802. Accordingly, actuator 1804 may be configured to rotate in the first direction when received along first threaded portion 1810, and in the second direction when received along second threaded portion 1820.

A longitudinal length of first threaded portion 1810 relative to barrel 1804 may correspond to the first volume of fluid substance, and a longitudinal length of second threaded portion 1820 relative to barrel 1804 may correspond to the second volume of fluid substance. Actuator 1804 may be configured to translate along the longitudinal lengths of first threaded portion 1810 and second threaded portion 1820 while rotating about barrel 1802 to dispense the first and second volumes of the fluid substance via needle 1806. It should be understood that the plunger disposed within the cavity of barrel 1802 and coupled to actuator 1804 may translates through an interior of barrel 1802 as actuator 1804 rotates along an exterior of barrel 1802.

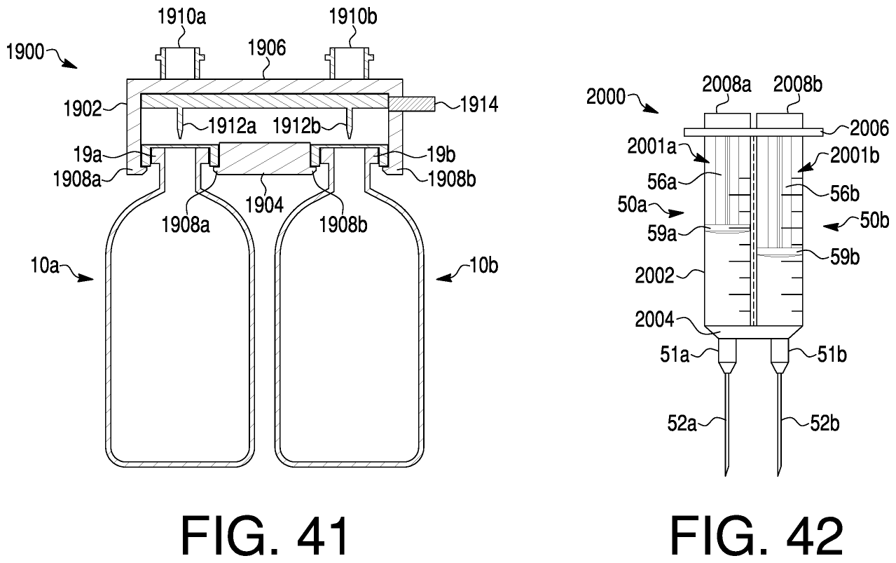

Referring now to FIG. 41, an exemplary vial adapter assembly 1900 is depicted. Vial adapter assembly 1900 may be configured and operable to retain and facilitate simultaneous access (fluid communication) to two or more vials housed together via respective connectors that may extend into each vial in response to actuating a single actuator. Vial adapter assembly 1900 may include a housing 1902 having a distal end 1904 and a proximal end 1906. Vial adapter assembly 1900 may further include a pair of first clips 1908a and a pair of second clips 1908b extending distally (e.g., downward) from distal end 1904. The pair of first clips 1908a may be configured to couple the first vial 10a to housing 1902 along the distal end 1904, and the pair of second clips 1908b may be configured to couple the second vial 10b to housing 1902 along the distal end 1904. In the example, the pair of first clips 1908a may be configured to engage the mouth 19a to form a snap-fit connection between housing 1902 and the first vial 10a, and the pair of second clips 1908b may be configured to engage the mouth 19b to form a snap-fit connection between housing 1902 and the second vial 10b.

A first cavity defined between the pair of first clips 1908a may correspond to a first receptacle within housing 1902 for receiving at least a portion of the first vial 10a, such as the mouth 19a. A second cavity defined between the pair of second clips 1908b may correspond to a second receptacle within housing 1902 for receiving at least a portion of the second vial 10b, such as the mouth 19b. Vial adapter assembly 1900 may further include a first connector 1910a and a second connector 1910b positioned proximate to the proximal end 1906, and a first needle 1912a and a second needle 1912b disposed within a cavity of housing 1902. In some embodiments, first connector 1910a and second connector 1910b may include a luer lock connector.

Still referring to FIG. 41, first connector 1910a may be fluidly coupled to first needle 1912a, and second connector 1910b may be fluidly coupled to second needle 1912b. In the example, first connector 1910a and first needle 1912a may be aligned with the first receptacle (defined between the pair of first clips 1908a) receiving the first vial 10a, and second connector 1910b and second needle 1912b may be aligned with the second receptacle (defined between the pair of second clips 1908b) for receiving the second vial 10b. Vial adapter assembly 1900 may include an actuator 1914 positioned along housing 1902, and actuator 1914 may be coupled to needles 1912a, 1912b.

In exemplary use, actuator 1914 may be configured to simultaneously move first needle 1912a and second needle 1912b relative to the cavity of housing 1902 in response to actuating actuator 1914. In this instance, needles 1912a, 1912b may translate distally (e.g., downward) toward the first and second receptacles, respectively. First needle 1912a may be configured to extend into the first vial 10a, thereby establishing fluid communication between the first vial 10a and first connector 1910a. Second needle 1912b may be configured to extend into the second vial 10b, thereby establishing fluid communication between the second vial 10b and second connector 1910b. Accordingly, an external device coupled to each of first connector 1910a and second connector 1910b may be fluidly coupled to the first vial 10a and the second vial 10b, respectively.

Referring now to FIG. 42, an exemplary adapter 2000 is depicted. Adapter 2000 may be configured and operable to retain and facilitate independent delivery of content stored within two or more syringes housed together via respective actuators coupled to each syringe. Adapter 2000 includes a housing 2002 having a longitudinal length defined between a distal end 2004 and a proximal end 2006. Housing 2002 may be sized and shaped to define a first receptacle 2001a for receiving the first syringe 50a, and a second receptacle 2001b for receiving the second syringe 50b. First syringe and second syringe 50a, 50b may be prefilled syringes. In the example, housing 2002 is configured to position the first syringe 50a and the second syringe 50b adjacent to one another and in parallel when received within first receptacle 2001 and second receptacle 2001b, respectively.

In the example, at least a portion of the first syringe 50a and the second syringe 50b may extend outwardly from housing 2002. For example, a distal end 51a and/or needle 52a of the first syringe 50a may be positioned external to first receptacle 2001a, such as to facilitate delivery of a first substance stored within the first syringe 50a outward from adapter 2000. Further, a distal end 51b and/or needle 52b of the second syringe 50b may be positioned external to second receptacle 2001b, such as to facilitate delivery of a second substance stored within the second syringe 50b outward from adapter 2000. Housing 2002 may be configured to receive at least a portion of the first syringe 50a and the second syringe 50b within housing 2002, such as the plunger rod 56a, 56b and the plunger 59a, 59b, respectively.

Still referring to FIG. 42, adapter 2000 may include a first actuator 2008a and a second actuator 2008b positioned proximate to the proximal end 2006. First actuator 2008a may be coupled to the plunger rod 56a of the first syringe 50a, and configured to translate the plunger rod 56a and the plunger 59a relative to housing 2002 for dispensing a fluid substance (e.g., a medicament) stored in the first syringe 50a.

Second actuator 2008b may be coupled to the plunger rod 56b of the second syringe 50b, and configured to translate the plunger rod 56b and the plunger 59b relative to housing 2002 for dispensing a second fluid substance (e.g., a medicament) stored in the second syringe 50b. Accordingly, adapter 2000 may be configured to allow delivery of the fluid substance stored in each syringe 50a, 50b independent of one another via the selective actuation of the respective actuator 2008a, 2008b.

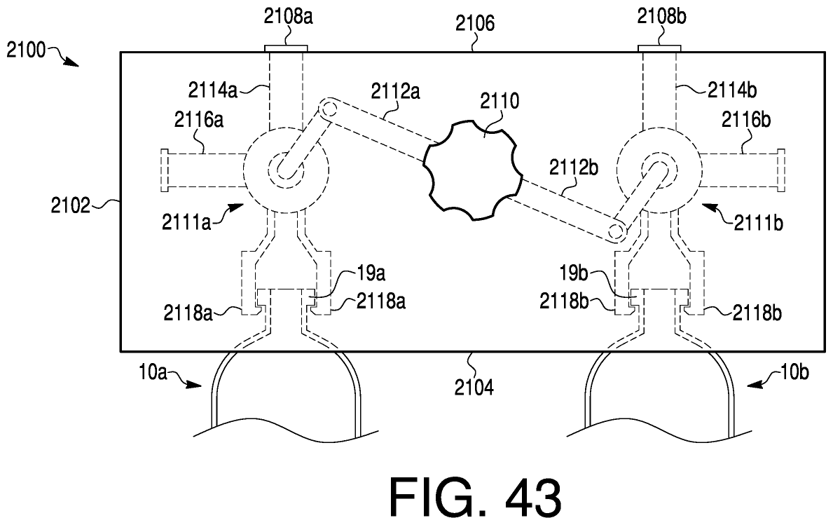

Referring now to FIG. 43, an exemplary vial adapter assembly 2100 is depicted. Vial adapter assembly 2100 may be configured and operable to retain and facilitate independent access (fluid communication) to two or more vials housed together via the actuation of a single actuator. Vial adapter assembly 2100 may include a housing 2102 having a distal end 2104 and a proximal end 2106. Housing 2102 may define a cavity enclosing a first valve assembly 2111a and a second valve assembly 2111b, each of which may be coupled to an actuator 2110 positioned along an exterior of housing 2102. First valve assembly 2111a and a second valve assembly 2111b may be, for example, stop cock valves. As described further below, actuator 2110 may be configured to transition valve assemblies 2111a, 2111b from a first configuration to a second configuration.

Valve adapter assembly 2100 may include a first connector 2108a and a second connector 2108b extending outwardly from housing 2102 proximate to the proximal end 2106. In the example, first connector 2108a may be in fluid communication with first valve assembly 2111a, and second connector 2108b may be in fluid communication with second valve assembly 2111b. In some embodiments, first connector 2108a and second connector 2108b may include a luer lock connector configured to couple an external device to vial adapter assembly 2100, and particularly the corresponding valve assembly 2111a, 2111b.

Still referring to FIG. 43, first valve assembly 2111a may include a first lever 2112a movably coupled to actuator 2110, a first channel 2114a fluidly coupled to first connector 2108a, and a second channel 2116a. Vial adapter assembly 2100 may further include a pair of first clips 2118a disposed within housing 2102 and configured to engage at least a portion of the first vial 10a, such as the mouth 19a, for securing the first vial 10a to first valve assembly 2111a. As described in further detail herein, the first vial 10a may be in fluid communication with at least one of the first channel 2114a and/or the second channel 2116a based on a relative position of actuator 2110.

Second valve assembly 2111b may include a second lever 2112b movably coupled to actuator 2110, a second channel 2114b fluidly coupled to second connector 2108b, and a second channel 2116b. Vial adapter assembly 2100 may further include a pair of second clips 2118b disposed within housing 2102 and configured to engage at least a portion of the second vial 10b, such as the mouth 19b, for securing the second vial 10b to second valve assembly 2111b. The second vial 10b may be in fluid communication with at least one of the first channel 2114b and/or the second channel 2116b based on a relative position of actuator 2110.

Still referring to FIG. 43, actuator 2110 may be configured to simultaneously adjust a fluid pathway of the first vial 10a and the second vial 10b to a respective channel of each valve assembly 2111a, 2111b. For example, actuator 2110 may be configured to cause first vial assembly 2111a to fluidly couple the first vial 10a to one of the first channel 2114a or the second channel 2116a. Simultaneously, actuator 2110 may be configured to cause second vial assembly 2111a to fluidly couple the second vial 10b to one of the first channel 2114b or the second channel 2116b. Accordingly, rotation of actuator 2110 may provide for a simultaneous actuation of first vial assembly 2111a and second vial assembly 2111b.

In some embodiments, first valve assembly 2111a may be configured to divert the fluid substance from the first vial 10a to the external device coupled to first connector 2108a when in fluid communication with first channel 2114a. Further, first valve assembly 2111a may be configured to divert the fluid substance away from the first vial 10a, such as to a closed, terminal end of second channel 2116a. Second valve assembly 2111b may be configured to divert the fluid substance from the second vial 10b to the external device coupled to second connector 2108b when in fluid communication with second channel 2114b. Further, second valve assembly 2111b may be configured to divert the fluid substance away from the second vial 10b, such as to a closed, terminal end of second channel 2116b. Each of first valve assembly 2111a and second valve assembly 2111b may be further configured to seal off fluid communication between both corresponding channels simultaneously. For example, a rotation of actuator 2110 to a predefined position may prevent each of first vial 10a and second vial 10b from being in fluid communication with either channels 2114, 2116.

In one embodiment, actuator 2110 may be configured to simultaneously close first valve assembly 2111a and second valve assembly 2111b when actuator 2110 is in a first rotative position, to open first valve assembly 2111a and close second valve assembly 2111b when in a second rotative position, and to close first valve assembly 2111a and open second valve assembly 2111b when in a third rotative position. It should be appreciated that each of the first, second, and third rotative positions may be relative to housing 2102 and respectively different from one another. In some embodiments, actuator 2110 may be configured to open both first valve assembly 2111a and second valve assembly 2111b when in a fourth rotative position.

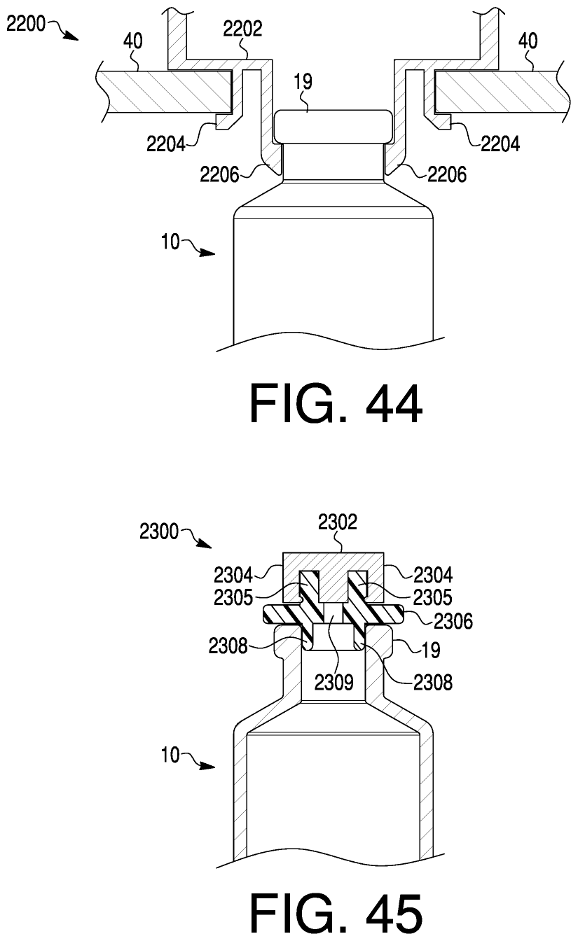

Referring now to FIG. 44, an exemplary vial adapter assembly 2200 is depicted. Vial adapter assembly 2200 may be configured and operable to facilitate access (fluid communication) to vials of different sizes. Vial adapter assembly 2200 may include a body 2202 having a pair of first clips 2204 and a pair of second clips 2206 positioned distally relative to the pair of first clips 2204. The pair of first clips 2204 may be configured to engage at least a portion of a sleeve 40, and the pair of second clips 2206 may be configured to engage at least a portion of the vial 10, such as the neck portion below the mouth 19.

Vial adapter assembly 2200 may be configured and operable to couple the sleeve 40 to the vial 10 having a particular size and/or shape. Vial adapter assembly 2200 may include a plurality of bodies having a pair of second clips that vary in size, shape, and/or configuration than that shown and described herein for coupling the sleeve 40 to different vials having various cross-sectional dimensions. Accordingly, vial adapter assembly 2200 may be configured to couple vials of varying sizes.

Still referring to FIG. 44, the pair of first clips 2204 may define a universal fastening pattern that is capable of attaching to various sleeves 40. The pair of second clips 2206 may be formed of plastic and operable to form a snap-fit connection with the shoulder 19 to secure the first vial 10 to the sleeve 40. In some embodiments, the sleeve 40 may include a portion of a syringe, a cartridge, a vial, and/or various other devices.

Referring now to FIG. 45, an exemplary crimp assembly 2300 is depicted. Crimp assembly 2300 may be configured and operable to facilitate access (fluid communication) to a vial through a crimp and stopper coupled thereto. Crimp assembly 2300 may include a crimp 2302 and a stopper 2306. Stopper 2306 may fit within an opening of vial 10 to control access to vial 10. Stopper 2306 may include one or more proximal arms 2305 extending proximally from a body of stopper 2306, and one or more distal arms 2308 extending distally from the body of stopper 2306 to secure stopper 2306 within vial 10. The one or more distal arms 2308 may be configured to mate with the vial 10, and particularly the mouth 19, to couple stopper 2306 to the vial 10. For example, the one or more distal arms 2308 may extend into the vial 10 through an opening at the mouth 19, thereby forming a seal against the opening of the vial 10. Stopper 2306 may be a flexible material, for example, rubber. An additional crimp (not shown) may also extend over the stopper 2306 to further secure it to vial 10. The additional crimp may be metal or plastic.

Crimp assembly 2300 may further include a crimp 2302 having a body and one or more arms 2304 extending distally from the body of crimp 2302. Crimp 2302 may be configured to cover stopper 2306 and seal off stopper 2306 when attached to stopper 2306. In the example, crimp 2302 may be at least partially flexible and formed of plastic. The one or more proximal arms 2305 of stopper 2306 may be configured to mate with the one or more arms 2304 of crimp 2302 to couple stopper 2306 to crimp 2302.

Still referring to FIG. 45, stopper 2306 may be configured to allow access into the vial 10, such as, for example, via a channel 2309 extending through the body of stopper 2306. With crimp 2302 coupled to stopper 2306, crimp 2302 may be configured to close channel 2309, thereby maintaining a sterility of the vial 10. Crimp 2302 may be selectively removable from stopper 2306 to facilitate access to the vial 10, such as, for example, by an external device (e.g., a syringe). Crimp assembly 2300 may eliminate the need for terminal sterilization prior to use with the external device.

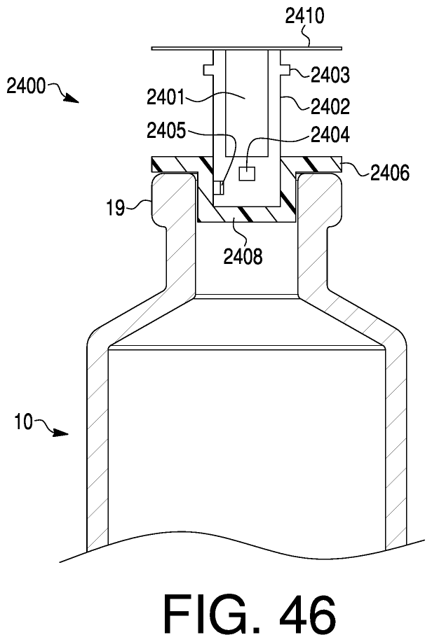

Referring now to FIG. 46, an exemplary crimp assembly 2400 is depicted. Crimp assembly 2400 may be configured and operable to facilitate access (fluid communication) to a vial through a connector capable of coupling an external device (e.g., a syringe) thereto. Crimp assembly 2400 may include a crimp 2402, a stopper 2406, and a peel tab 2410. Crimp 2402 may include a body having a connector 2403 at a proximal end and a plurality of openings 2404, 2405 at a distal end. In the example, crimp 2402 may include at least a first opening 2404 and a second opening 2405. A position of first opening 2404 relative to the body of crimp 2402 may be laterally and/or longitudinally offset from a position of second opening 2406 on the body of crimp 2402. In other embodiments, crimp 2402 may include additional openings along the distal end than those shown and described herein, or may include one opening.

The body of crimp 2402 may further define a channel 2401 extending along the proximal end of crimp 2402, with each of the plurality of openings 2404, 2405 being in fluid communication with the channel 2401. An opening of the channel 2401 at the proximal end of crimp 2402 may be covered by peel tab 2410 coupled thereto. Peel tab 2410 may be configured to form a seal against crimp 2402 and close the opening of the channel 2401, thereby maintaining a sterility of crimp 2402. As described herein, channel 2401 and openings 2404, 2405 may be configured to facilitate a fluid communication between an external device and the vial 10.

Still referring to FIG. 46, connector 2403 may include a luer lock connector configured to couple crimp 2402 to an external device, such as a syringe, upon removal of peel tab 2410 from the proximal end of crimp 2402. Stopper 2406 may include a body 2408 that is configured to extend into the vial 10, such as through an opening at the mouth 19. In this instance, stopper 2406 may be configured to form a seal against the mouth 19, thereby maintaining a sterility of the vial 10. The body 2408 may be sized and shaped to receive at least a portion of crimp 2402, such as the distal end of crimp 2402. In this instance, the plurality of openings 2404, 2405 may be at least partially disposed within the body 2408.

In a first configuration, crimp 2402 may be positioned relative to stopper 2406 such that one or more of first opening 2404 and second opening 2405 may be sealed against body 2408 such that the channel 2401 is not in fluid communication with the vial 10. Crimp 2402 may be configured to move (e.g., rotate) relative to stopper 2406 from the first configuration to a second configuration, such as, for example, in response to a simultaneous movement of the external device coupled to crimp 2402 via connector 2403.

In the second configuration, first opening 2404 and second opening 2405 may be repositioned relative to stopper 2406 such that body 2408 does not abut against one or more of first opening 2404 and/or second opening 2405. In this instance, crimp 2402 may be configured to facilitate fluid communication between the external device and the vial 10 via openings 2404, 2405 and channel 2401.

Features enumerated above have been described within the context of particular embodiments. However, as one of ordinary skill in the art would understand, features and aspects of each embodiment may be combined, added to other embodiments, subtracted from an embodiment, etc. in any manner suitable to assist with controlled preparation and/or delivery of a drug.

Aspects of the embodiments disclosed herein are described with respect to assembling multiple vials and/or cartridges storing different fluid substances (e.g., medicament) to one another, and some embodiments disclosed herein are described as being particular types of drug delivery devices (e.g., syringes, pipettes, crimps). Aspects of the present disclosure may also be employed and/or used with other types of devices.

While a number of embodiments are presented herein, multiple variations on such embodiments, and combinations of elements from one or more embodiments, are possible and are contemplated to be within the scope of the present disclosure. Moreover, those skilled in the art will appreciate that the conception upon which this disclosure is based may readily be used as a basis for designing other devices, methods, and systems for carrying out the several purposes of the present disclosure.

Embodiments of the present disclosure may include the following features:

Item 1. A vial adapter assembly, comprising:

a housing;

a first receptacle formed within the housing and configured to at least partially receive a first vial within a body of the housing, wherein an opening of the first receptacle is in fluid communication with an upper surface of the housing;

a second receptacle formed within the housing, and configured to at least partially receive a second vial within the body of the housing, wherein an opening of the second receptacle is in fluid communication with the upper surface of the housing; and

a cap coupled to the housing, wherein the cap is movable relative to the housing from a closed configuration to an open configuration;

wherein, in the closed configuration, the cap is configured to cover the opening of the first receptacle and the opening of the second receptacle on the upper surface of the housing; and

wherein, in the open configuration, the cap is configured to uncover the opening of the first receptacle and the opening of the second receptacle, and expose the upper surface of the housing.

Item 2. The vial adapter assembly of item 1, wherein the housing includes at least one slot on the upper surface of the housing, and the cap includes at least one corresponding protrusion extending outwardly from a surface of the cap configured to mate with the upper surface of the housing.

Item 3. The vial adapter assembly of item 2, wherein, in the closed configuration, each of the at least one slots is configured to receive one of the at least one corresponding protrusions.

Item 4. The vial adapter assembly of item 2, wherein, in the open configuration, each of the at least one protrusions is configured to deform, such that the cap is inhibited from transitioning from the open configuration to the closed configuration.

Item 5. The vial adapter assembly of item 1, wherein the first receptacle incudes a second opening along a lower portion of the housing, and the second receptacle incudes a second opening along the lower portion;

wherein the first openings of the first and second receptacles are sized smaller relative to the second openings of the first and second receptacles.

Item 6. The vial adapter assembly of item 5, wherein the second openings of the first receptacle and the second receptacle are sized to receive the first vial and the second vial, respectively; and

wherein the first openings of the first receptacle and the second receptacle are positioned to align with first and second openings of the first and second vial, respectively, when the first and second vial are received within the first and second receptacles.

Item 7. The vial adapter assembly of item 1, wherein the first receptacle is positioned adjacent to the second receptacle such that the first vial is maintained alongside the second vial when received within the housing.

Item 8. The vial adapter assembly of item 1, further including a single hinge coupling the housing to the cap, wherein the hinge is configured to pivot the cap relative to the housing when moving from the closed configuration to the open configuration.

Item 9. The vial adapter assembly of item 1, wherein the cap is configured to simultaneously cover the opening of the first receptacle and the opening of the second receptacle when in the closed configuration, and simultaneously uncover the opening of the first receptacle and the opening of the second receptacle when in the open configuration.

Item 10. The vial adapter assembly of item 1, wherein the housing is configured to at least partially enclose the first vial and the second vial such that the first vial and the second vial are positioned adjacent to one another.

Item 11. The vial adapter assembly of item 1, further including an actuator positioned over an opening of the first receptacle and an opening of the second receptacle.

Item 12. The vial adapter assembly of item 11, wherein the actuator includes a first hollow piercing element extending towards the first receptacle and a second hollow piercing element extending towards the second receptacle.

Item 13. The vial adapter assembly of item 12, wherein the actuator is configured to simultaneously move the first hollow piercing element relative to the first receptacle and the second hollow piercing element relative to the second receptacle.

Item 14. A vial adapter assembly, comprising:

a housing;

a first receptacle formed within the housing, and configured to at least partially receive a first vial;

a second receptacle formed within the housing, and configured to at least partially receive a second vial;

a first actuator coupled to the housing and positioned in longitudinal alignment with the first receptacle, such that the first actuator is positioned over an opening of the first receptacle, and wherein the first actuator includes a hollow piercing element extending towards the first receptacle; and

a second actuator coupled to the housing and positioned in longitudinal alignment with the second receptacle, such that the second actuator is positioned over an opening of the second receptacle, and wherein the second actuator includes a hollow piercing element extending towards the second receptacle;

wherein each of the first actuator and the second actuator are independently movable relative to the housing between an extended configuration and a compressed configuration;

wherein, in the extended configuration, the piercing element of the first actuator and the piercing element of the second actuator are positioned outside of the first receptacle and second receptacle, respectively, such that the piercing element of the first actuator and the piercing element of the second actuator are not in fluid communication with the first receptacle and the second receptacle; and

wherein, in the compressed configuration, the piercing element of the first actuator and the piercing element of the second actuator are positioned at least partially within the first receptacle and the second receptacle, respectively, such that the piercing element of the first actuator and the piercing element of the second actuator are in fluid communication with the first receptacle and the second receptacle, respectively.

Item 15. The vial adapter assembly of item 14, further including a first removable barrier coupled to the housing and positioned between the first actuator and the first receptacle; and

a second removable barrier coupled to the housing and positioned between the second actuator and the second receptacle.

Item 16. The vial adapter assembly of item 15, wherein the first removable barrier and the second removable barrier include an adhesive liner.

Item 17. The vial adapter assembly of item 12, wherein the housing includes a first slot and a second slot, and the first slot and the second slot are sized to receive at least one of the first removable barrier and the second removable barrier.

Item 18. The vial adapter assembly of item 17, wherein the first removable barrier is configured to inhibit movement of the first actuator from the extended configuration to the compressed configuration when the first removable barrier is received within the first slot; and

wherein the second removable barrier is configured to inhibit movement of the second actuator from the extended configuration to the compressed configuration when the second removable barrier is received within the second slot.

Item 19. The vial adapter assembly of item 18, wherein the first removable barrier and the second removable barrier are configured to be independently removable relative to the housing.

Item 20. The vial adapter assembly of item 14, wherein each of the first actuator and the second actuator include a luer lock connector.

Item 21. A vial adapter assembly, comprising:

a housing;

a first receptacle formed within the housing, and configured to at least partially receive a first vial,

a first connector fixed to the housing and positioned at least partially within the first receptacle so that a distal end of the first connector extends within and is in fluid communication with the first vial when received within the first receptacle;

a second receptacle formed within the housing, and configured to at least partially receive a second vial;

a second connector fixed to the housing and positioned at least partially within the second receptacle so that a distal end of the second connector extends within and is in fluid communication with the second vial when received within the second receptacle;

a first actuator coupled to the housing and positioned in longitudinal alignment with the first receptacle; and

a second actuator coupled to the housing and positioned in longitudinal alignment with the second receptacle, wherein each of the first actuator and the second actuator are independently movable relative to the housing between an extended configuration and a compressed configuration;

wherein, when the first actuator and the second actuator are in the extended configuration, the first actuator and the second actuator conceal a proximal region of the first connector and the second connector, respectively; and

wherein, when the first actuator and the second actuator are in the compressed configuration, the first actuator and the second actuator expose the proximal region of the first connector and the second connector, respectively.

Item 22. The vial adapter assembly of item 21, further including a first removable barrier coupled to the first actuator, and a second removable barrier coupled to the second actuator when the first actuator and the second actuator are in the extended configuration.

Item 23. The vial adapter assembly of item 22, wherein the first removable barrier and the second removable barrier include at least one of an adhesive liner or a cap.

Item 24. The vial adapter assembly of item 22, wherein the first removable barrier is positioned over a proximal end of the first connector when coupled to the first actuator, and the second removable barrier is positioned over a proximal end of the second connector when coupled to the second actuator.

Item 25. The vial adapter assembly of item 22, further including a first lock-out mechanism configured to prevent the first actuator from returning from the compressed configuration to the extended configuration, and a second lock-out mechanism configured to prevent the second actuator from returning from the compressed configuration to the extended configuration.

Item 26. The vial adapter assembly of item 21, wherein the proximal region of the first connector extends outwardly from the first actuator when the first actuator is in the compressed configuration, and the proximal region of the second connector extends outwardly from the second actuator when the second actuator is in the compressed configuration.

Item 27. The vial adapter assembly of item 26, wherein the first removable barrier and the second removable barrier are configured to be independently movable relative to the housing.

Item 28. The vial adapter assembly of item 21, wherein the proximal region of each of the first connector and the second connector include a luer lock.

Item 29. A vial adapter assembly, comprising:

a housing;

a first receptacle formed within the housing, wherein the first receptacle is configured to receive a first vial therein;

a first connector movably coupled to the housing and positioned at least partially within the first receptacle, the first connector includes a first channel defining a first flow path;

a second receptacle formed within the housing, wherein the second receptacle is configured to receive a second vial therein;

a second connector movably coupled to the housing and positioned at least partially within the second receptacle, the second connector includes a second channel defining a second flow path;

wherein the first connector and the second connector are each configured to independently move relative to the housing between a first configuration and a second configuration;

wherein, in the first configuration, the first flow path is obstructed, and the second flow path is obstructed; and

wherein, in the second configuration, the first flow path is unobstructed, and the second flow path is unobstructed.

Item 30. The vial adapter assembly of item 29, further including a first stopper extending around a distal region of the first connector within the first receptacle, and a second stopper extending around a distal region of the second connector within the second receptacle.

Item 31. The vial adapter assembly of item 29, wherein the first flow path extends through a sidewall of a distal region of the first connector, and wherein the second flow path extends through a sidewall of a distal region of the second connector.

Item 32. The vial adapter assembly of item 31, further including a first stopper extending around the distal region of the first connector within the first receptacle, and a second stopper extending around the distal region of the second connector within the second receptacle, wherein a portion of the first stopper is configured to obstruct the first flow path when the first connector is in the first configuration, and the second stopper is configured to obstruct the second flow path when the second connector is in the first configuration.

Item 33. The vial adapter assembly of item 32, wherein the first connector is configured to rotate relative to the housing when transitioning from the first configuration to the second configuration; and

wherein the second connector is configured to rotate relative to the housing when transitioning from the first configuration to the second configuration.

Item 34. The vial adapter assembly of item 29, wherein the first connector and the second connector are each configured to independently rotate relative to the housing between the first configuration and the second configuration.

Item 35. The vial adapter assembly of item 29, wherein each of the first connector and the second connector include a proximal region that extends proximately out from the housing, and is configured to be independently movable relative to the housing.

Item 36. The vial adapter assembly of item 29, wherein each of the first connector and the second connector include a proximal region configured to couple to an external device.

Item 37. The vial adapter assembly of item 36, wherein the proximal region configured to couple to the external device includes a luer lock.

Item 38. A vial adapter assembly, comprising:

a housing;

a first receptacle formed within the housing and configured to at least partially receive a first vial within a body of the housing, wherein an opening of the first receptacle is in fluid communication with an upper surface of the housing;

a cap coupled to the housing, wherein the cap includes a hollow piercing element and the cap is movable relative to the housing from a closed configuration to an open configuration;

wherein, in the closed configuration, the cap is configured to cover the opening of the first receptacle on the upper surface of the housing; and

wherein, in the open configuration, the cap is configured to uncover the opening of the first receptacle and expose the upper surface of the housing; and

an external device coupled to the housing.

Item 39. The vial adapter assembly of item 38, including a second receptacle formed within the housing, and configured to at least partially receive a second vial within the body of the housing, wherein an opening of the second receptacle is in fluid communication with the upper surface of the housing.

Item 40. The vial adapter assembly of item 38, wherein the hollow piercing element extends distally from an interior surface of the cap.

Item 41. The vial adapter assembly of item 38, wherein the cap includes a proximal region configured to couple to the external device.

Item 42. The vial adapter assembly of item 40, wherein the proximal region configured to couple to the external device includes a connector extending proximally from an exterior surface of the cap.

Item 43. The vial adapter assembly of item 42, wherein the connector is a luer lock.

Item 44. The vial adapter assembly of item 38, wherein the external device is a syringe or an IV bag.

Item 45. A vial adapter assembly, comprising:

a housing;

a first cartridge within the housing, the first cartridge containing a first substance;

a second cartridge within the housing, the second cartridge containing a second substance;

a first actuator coupled to the housing and positioned in longitudinal alignment with the first cartridge;

a second actuator coupled to the housing and positioned in longitudinal alignment with the second cartridge, wherein each of the first actuator and the second actuator are independently movable relative to the housing between an extended configuration and a compressed configuration,

wherein, in the extended configuration, a portion of the first actuator extends

outwardly from the housing in a proximal direction and a portion of the second actuator extends outwardly from the housing in a proximal direction, and

wherein, in the compressed configuration, a portion of the first actuator is concealed within the first cartridge and a portion of the second actuator is concealed within the second cartridge.

Item 46. The vial adapter assembly of item 45, including a third cartridge within the housing, the third cartridge containing a third substance, and a third actuator coupled to the housing and in longitudinal alignment with the third cartridge.

Item 47. The vial adapter assembly of item 46, wherein, in the extended configuration, a portion of the third actuator extends outwardly from the housing in a proximal direction and in the compressed configuration, a portion of the third actuator is concealed within the third cartridge.

Item 48. The vial adapter assembly of item 47, wherein the housing includes a proximal end and a distal tip.

Item 49. The vial adapter assembly of item 48, wherein a first actuation translates a distal end of the respective cartridge towards the distal tip of the housing and a second actuation delivers the substance stored within the respective cartridge.

Item 50. The vial adapter assembly of item 49, wherein the first actuation translates the distal end of the respective cartridge outward from the housing via an opening of the distal tip.

Item 51. The vial adapter assembly of item 45, wherein the distal tip of the housing is configured to connect to an external device.

Item 52. The vial adapter assembly of item 51, wherein the distal tip includes a luer lock connection.

Item 53. The vial adapter assembly of item 51, wherein the external device is a syringe or an IV bag.

Item 54. The vial adapter assembly of item 45, wherein the housing includes a first receptacle including the first cartridge and a second receptacle including the second cartridge.

Item 55. The vial adapter assembly of item 54, wherein the first cartridge is a first syringe and the second cartridge is a second syringe.

Item 56. The vial adapter assembly of item 55, wherein the first syringe includes a first needle extending from a distal end of the first syringe and the second syringe includes a first needle extending from a distal end of the second syringe.

What is claimed is:

1. A vial adapter assembly, comprising:
a housing;
a first receptacle formed within the housing and configured to at least partially receive a first vial within a body of the housing, wherein an opening of the first receptacle is in fluid communication with an upper surface of the housing;
a second receptacle formed within the housing, and configured to at least partially receive a second vial within the body of the housing, wherein an opening of the second receptacle is in fluid communication with the upper surface of the housing; and
a cap coupled to the housing, wherein the cap is movable relative to the housing from a closed configuration to an open configuration;
wherein, in the closed configuration, the cap is configured to cover the opening of the first receptacle and the opening of the second receptacle on the upper surface of the housing; and
wherein, in the open configuration, the cap is configured to uncover the opening of the first receptacle and the opening of the second receptacle, and expose the upper surface of the housing.

2. The vial adapter assembly of claim 1, wherein the housing includes at least one slot on the upper surface of the housing, and the cap includes at least one corresponding protrusion extending outwardly from a surface of the cap configured to mate with the upper surface of the housing.

3. The vial adapter assembly of claim 2, wherein, in the closed configuration, each of the at least one slots is configured to receive one of the at least one corresponding protrusions.

4. The vial adapter assembly of claim 2, wherein, in the open configuration, each of the at least one protrusions is configured to deform, such that the cap is inhibited from transitioning from the open configuration to the closed configuration.

5. The vial adapter assembly of claim 1, wherein the first receptacle incudes a second opening along a lower portion of the housing, and the second receptacle incudes a second opening along the lower portion;
wherein the first openings of the first and second receptacles are sized smaller relative to the second openings of the first and second receptacles.

6. The vial adapter assembly of claim 5, wherein the second openings of the first receptacle and the second receptacle are sized to receive the first vial and the second vial, respectively; and
wherein the first openings of the first receptacle and the second receptacle are positioned to align with first and second openings of the first and second vial, respectively, when the first and second vial are received within the first and second receptacles.

7. The vial adapter assembly of claim 1, wherein the first receptacle is positioned adjacent to the second receptacle such that the first vial is maintained alongside the second vial when received within the housing.

8. The vial adapter assembly of claim 1, further including a single hinge coupling the housing to the cap, wherein the hinge is configured to pivot the cap relative to the housing when moving from the closed configuration to the open configuration.

9. The vial adapter assembly of claim 1, wherein the cap is configured to simultaneously cover the opening of the first receptacle and the opening of the second receptacle when in the closed configuration, and simultaneously uncover the opening of the first receptacle and the opening of the second receptacle when in the open configuration.

10. The vial adapter assembly of claim 1, wherein the housing is configured to at least partially enclose the first vial and the second vial such that the first vial and the second vial are positioned adjacent to one another.

11. The vial adapter assembly of claim 1, further including an actuator positioned over an opening of the first receptacle and an opening of the second receptacle.

12. The vial adapter assembly of claim 11, wherein the actuator includes a first hollow piercing element extending towards the first receptacle and a second hollow piercing element extending towards the second receptacle.

13. The vial adapter assembly of claim 12, wherein the actuator is configured to simultaneously move the first hollow piercing element relative to the first receptacle and the second hollow piercing element relative to the second receptacle.

14. A vial adapter assembly, comprising:
a housing;
a first receptacle formed within the housing, and configured to at least partially receive a first vial;
a second receptacle formed within the housing, and configured to at least partially receive a second vial;
a first actuator coupled to the housing and positioned in longitudinal alignment with the first receptacle, such that the first actuator is positioned over an opening of the first receptacle, and wherein the first actuator includes a hollow piercing element extending towards the first receptacle; and
a second actuator coupled to the housing and positioned in longitudinal alignment with the second receptacle, such that the second actuator is positioned over an opening of the second receptacle, and wherein the second actuator includes a hollow piercing element extending towards the second receptacle;
wherein each of the first actuator and the second actuator are independently movable relative to the housing between an extended configuration and a compressed configuration;
wherein, in the extended configuration, the piercing element of the first actuator and the piercing element of the second actuator are positioned outside of the first receptacle and second receptacle, respectively, such that the piercing element of the first actuator and the piercing element of the second actuator are not in fluid communication with the first receptacle and the second receptacle; and
wherein, in the compressed configuration, the piercing element of the first actuator and the piercing element of the second actuator are positioned at least partially within the first receptacle and the second receptacle, respectively, such that the piercing element of the first actuator and the piercing element of the second actuator are in fluid communication with the first receptacle and the second receptacle, respectively.

15. The vial adapter assembly of claim 14, further including a first removable barrier coupled to the housing and positioned between the first actuator and the first receptacle; and
a second removable barrier coupled to the housing and positioned between the second actuator and the second receptacle.

16. The vial adapter assembly of claim 15, wherein the first removable barrier and the second removable barrier include an adhesive liner.

17. The vial adapter assembly of claim 12, wherein the housing includes a first slot and a second slot, and the first slot and the second slot are sized to receive at least one of the first removable barrier and the second removable barrier.

18. The vial adapter assembly of claim 17, wherein the first removable barrier is configured to inhibit movement of the first actuator from the extended configuration to the compressed configuration when the first removable barrier is received within the first slot; and
wherein the second removable barrier is configured to inhibit movement of the second actuator from the extended configuration to the compressed configuration when the second removable barrier is received within the second slot.

19. The vial adapter assembly of claim 18, wherein the first removable barrier and the second removable barrier are configured to be independently removable relative to the housing.

20. The vial adapter assembly of claim 14, wherein each of the first actuator and the second actuator include a luer lock connector.

21. A vial adapter assembly, comprising:
a housing;
a first receptacle formed within the housing, and configured to at least partially receive a first vial,
a first connector fixed to the housing and positioned at least partially within the first receptacle so that a distal end of the first connector extends within and is in fluid communication with the first vial when received within the first receptacle;
a second receptacle formed within the housing, and configured to at least partially receive a second vial;
a second connector fixed to the housing and positioned at least partially within the second receptacle so that a distal end of the second connector extends within and is in fluid communication with the second vial when received within the second receptacle;
a first actuator coupled to the housing and positioned in longitudinal alignment with the first receptacle; and
a second actuator coupled to the housing and positioned in longitudinal alignment with the second receptacle, wherein each of the first actuator and the second actuator are independently movable relative to the housing between an extended configuration and a compressed configuration;
wherein, when the first actuator and the second actuator are in the extended configuration, the first actuator and the second actuator conceal a proximal region of the first connector and the second connector, respectively; and
wherein, when the first actuator and the second actuator are in the compressed configuration, the first actuator and the second actuator expose the proximal region of the first connector and the second connector, respectively.

22. The vial adapter assembly of claim 21, further including a first removable barrier coupled to the first actuator, and a second removable barrier coupled to the second actuator when the first actuator and the second actuator are in the extended configuration.

23. The vial adapter assembly of claim 22, wherein the first removable barrier and the second removable barrier include at least one of an adhesive liner or a cap.

24. The vial adapter assembly of claim 22, wherein the first removable barrier is positioned over a proximal end of the first connector when coupled to the first actuator, and the second removable barrier is positioned over a proximal end of the second connector when coupled to the second actuator.

25. The vial adapter assembly of claim 22, further including a first lock-out mechanism configured to prevent the first actuator from returning from the compressed configuration to the extended configuration, and a second lock-out mechanism configured to prevent the second actuator from returning from the compressed configuration to the extended configuration.

26. The vial adapter assembly of claim 21, wherein the proximal region of the first connector extends outwardly from the first actuator when the first actuator is in the compressed configuration, and the proximal region of the second connector extends outwardly from the second actuator when the second actuator is in the compressed configuration.

27. The vial adapter assembly of claim 26, wherein the first removable barrier and the second removable barrier are configured to be independently movable relative to the housing.

28. The vial adapter assembly of claim 21, wherein the proximal region of each of the first connector and the second connector include a luer lock.

29. A vial adapter assembly, comprising:
a housing;
a first receptacle formed within the housing, wherein the first receptacle is configured to receive a first vial therein;
a first connector movably coupled to the housing and positioned at least partially within the first receptacle, the first connector includes a first channel defining a first flow path;
a second receptacle formed within the housing, wherein the second receptacle is configured to receive a second vial therein;
a second connector movably coupled to the housing and positioned at least partially within the second receptacle, the second connector includes a second channel defining a second flow path;
wherein the first connector and the second connector are each configured to independently move relative to the housing between a first configuration and a second configuration;
wherein, in the first configuration, the first flow path is obstructed, and the second flow path is obstructed; and
wherein, in the second configuration, the first flow path is unobstructed, and the second flow path is unobstructed.

30. The vial adapter assembly of claim 29, further including a first stopper extending around a distal region of the first connector within the first receptacle, and a second stopper extending around a distal region of the second connector within the second receptacle.

31. The vial adapter assembly of claim 29, wherein the first flow path extends through a sidewall of a distal region of the first connector, and wherein the second flow path extends through a sidewall of a distal region of the second connector.

32. The vial adapter assembly of claim 31, further including a first stopper extending around the distal region of the first connector within the first receptacle, and a second stopper extending around the distal region of the second connector within the second receptacle, wherein a portion of the first stopper is configured to obstruct the first flow path when the first connector is in the first configuration, and the second stopper is configured to obstruct the second flow path when the second connector is in the first configuration.

33. The vial adapter assembly of claim 32, wherein the first connector is configured to rotate relative to the housing when transitioning from the first configuration to the second configuration; and
wherein the second connector is configured to rotate relative to the housing when transitioning from the first configuration to the second configuration.

34. The vial adapter assembly of claim 29, wherein the first connector and the second connector are each configured to independently rotate relative to the housing between the first configuration and the second configuration.

35. The vial adapter assembly of claim 29, wherein each of the first connector and the second connector include a proximal region that extends proximately out from the housing, and is configured to be independently movable relative to the housing.

36. The vial adapter assembly of claim 29, wherein each of the first connector and the second connector include a proximal region configured to couple to an external device.

37. The vial adapter assembly of claim 36, wherein the proximal region configured to couple to the external device includes a luer lock.

38. A vial adapter assembly, comprising:
a housing;
a first receptacle formed within the housing and configured to at least partially receive a first vial within a body of the housing, wherein an opening of the first receptacle is in fluid communication with an upper surface of the housing;
a cap coupled to the housing, wherein the cap includes a hollow piercing element and the cap is movable relative to the housing from a closed configuration to an open configuration;
wherein, in the closed configuration, the cap is configured to cover the opening of the first receptacle on the upper surface of the housing; and
wherein, in the open configuration, the cap is configured to uncover the opening of the first receptacle and expose the upper surface of the housing; and
an external device coupled to the housing.

39. The vial adapter assembly of claim 38, including a second receptacle formed within the housing, and configured to at least partially receive a second vial within the body of the housing, wherein an opening of the second receptacle is in fluid communication with the upper surface of the housing.

40. The vial adapter assembly of claim 38, wherein the hollow piercing element extends distally from an interior surface of the cap.

41. The vial adapter assembly of claim 38, wherein the cap includes a proximal region configured to couple to the external device.

42. The vial adapter assembly of claim 40, wherein the proximal region configured to couple to the external device includes a connector extending proximally from an exterior surface of the cap.

43. The vial adapter assembly of claim 42, wherein the connector is a luer lock.

44. The vial adapter assembly of claim 38, wherein the external device is a syringe or an IV bag.

45. A vial adapter assembly, comprising:
a housing;
a first cartridge within the housing, the first cartridge containing a first substance;
a second cartridge within the housing, the second cartridge containing a second substance;
a first actuator coupled to the housing and positioned in longitudinal alignment with the first cartridge;
a second actuator coupled to the housing and positioned in longitudinal alignment with the second cartridge, wherein each of the first actuator and the second actuator are independently movable relative to the housing between an extended configuration and a compressed configuration,
wherein, in the extended configuration, a portion of the first actuator extends outwardly from the housing in a proximal direction and a portion of the second actuator extends outwardly from the housing in a proximal direction, and
wherein, in the compressed configuration, a portion of the first actuator is concealed within the first cartridge and a portion of the second actuator is concealed within the second cartridge.

46. The vial adapter assembly of claim 45, including a third cartridge within the housing, the third cartridge containing a third substance, and a third actuator coupled to the housing and in longitudinal alignment with the third cartridge.

47. The vial adapter assembly of claim 46, wherein, in the extended configuration, a portion of the third actuator extends outwardly from the housing in a proximal direction and in the compressed configuration, a portion of the third actuator is concealed within the third cartridge.

48. The vial adapter assembly of claim 47, wherein the housing includes a proximal end and a distal tip.

49. The vial adapter assembly of claim 48, wherein a first actuation translates a distal end of the respective cartridge towards the distal tip of the housing and a second actuation delivers the substance stored within the respective cartridge.

50. The vial adapter assembly of claim 49, wherein the first actuation translates the distal end of the respective cartridge outward from the housing via an opening of the distal tip.

51. The vial adapter assembly of claim 45, wherein the distal tip of the housing is configured to connect to an external device.

52. The vial adapter assembly of claim 51, wherein the distal tip includes a luer lock connection.

53. The vial adapter assembly of claim 51, wherein the external device is a syringe or an IV bag.

54. The vial adapter assembly of claim 45, wherein the housing includes a first receptacle including the first cartridge and a second receptacle including the second cartridge.

55. The vial adapter assembly of claim 54, wherein the first cartridge is a first syringe and the second cartridge is a second syringe.

56. The vial adapter assembly of claim 55, wherein the first syringe includes a first needle extending from a distal end of the first syringe and the second syringe includes a first needle extending from a distal end of the second syringe.
